(19) United States
(12) Patent Application Publication
(10) Pub. No.: US 2024/0009168 A1
(43) Pub. Date: Jan. 11, 2024

(54) COMPOSITIONS FOR TREATING DRY AGE-RELATED MACULAR DEGENERATION (AMD)

(71) Applicant: Cognition Therapeutics, Inc., Pittsburgh, PA (US)

(72) Inventors: Anthony CAGGIANO, Pittsburgh, PA (US); Susan CATALANO, South San Francisco, CA (US); Mary HAMBY, Pittsburgh, PA (US); Nicholas IZZO, Pittsburgh, PA (US); Gary LOOK, Santa Clara, CA (US); Gilbert RISHTON, Los Angeles, CA (US)

(73) Assignee: Cognition Therapeutics, Inc., Pittsburgh, PA (US)

(21) Appl. No.: 18/253,071

(22) PCT Filed: Dec. 10, 2021
(86) PCT No.: PCT/US21/62850

§ 371 Date: May 16, 2023

Related U.S. Application Data
(60) Provisional application No. 63/124,695, filed on Dec. 11, 2020.

(51) Int. Cl. A61K 31/4035; A61K 31/453; A61K 31/445; A61P 27/02

(52) U.S. Cl. CPC A61K 31/4035; A61P 27/02; A61K 31/445; A61K 31/453

(57) ABSTRACT

The present disclosure relates to methods of treating dry age-related macular degeneration (dry AMD), comprising administering to a subject in need thereof, a therapeutically effective amount of a compound or pharmaceutical composition according to any embodiment described herein.

### CROSS-REFERENCE TO RELATED APPLICATIONS

This Application claims priority to U.S. Provisional Application No. 63/124,695 filed on Dec. 11, 2020, which is incorporated herein by reference.

### SUMMARY

The disclosure provides methods of treating dry age-related macular degeneration (dry AMD) and other retinal diseases, comprising administering to a subject in need thereof, a therapeutically effective amount of a compound or pharmaceutical composition according to any embodiment described herein.

Some embodiments describe a method of treating dry age-related macular degeneration (dry AMD), comprising administering to a subject in need thereof, a therapeutically effective amount of a compound selected from the group consisting of:

A. a compound of Formula I,

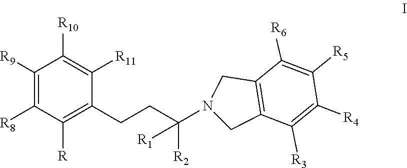

or a pharmaceutically-acceptable salt thereof:
wherein:
each of R₁ and R₂ is independently selected from H, C₁-C₆ alkyl, or CH₂OR′; wherein each R′ if present in R₁, and R₂ is independently H or C₁-C₆ alkyl; each of R₃, R₄, R₅, and R₆ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁-C₆ alkoxy C₁-C₆alkyl, aryl, heteroaryl, C₃-C₇ cycloalkyl, heterocycloalkyl, alkylaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₃, R₄, R₅, and R₆ is independently selected from the group consisting of H, CH₃, CH₂CH₃, C₃-C₆ alkyl, C₁-C₆ haloalkyl, or optionally substituted aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂, wherein the optionally substituted group is selected from C₁-C₆ alkyl or C₂-C₇ acyl;
or R₃ and R₄, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆haloalkyl, C₁-C₆ alkoxy, C₁-C₆haloalkoxy, aryl, arylalkyl, heteroaryl, heteroarylalkyl, cycloalkyl, and heterocycloalkyl or R₃ and R₄ are linked together to form a —O—C₁-C₂ methylene-O— group;
or R₄ and R₅, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆haloalkyl, C₁-C₆ alkoxy, C₁-C₆haloalkoxy, aryl, arylalkyl, heteroaryl, heteroarylalkyl, cycloalkyl, and heterocycloalkyl or R₄ and R₅ are linked together to form a —O—C₁₋₂ methylene-O— group;
each of R₇, R₈, R₉, R₁₀, and R₁₁ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁-C₆ alkoxy C₁-C₆ alkyl, aryl, heteroaryl, C₃-C₇ cycloalkyl, heterocycloalkyl, alkylaryl, heteroaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₇, R₈, R₉, R₁₀, and R₁₁ is independently selected from the group consisting of H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂;
or R₇ and R₈, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₇ and R₈ are linked together to form a —O—C₁₋₂ methylene-O— group;
or R₈ and R₉, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₈ and R₉ are linked together to form a —O—C₁₋₂ methylene-O— group;
each n is independently 0, 1, or 2;
with the proviso that R₇, R₈, R₉, R₁₀, and R₁₁ are not all H; and with the proviso that the following compounds, or pharmaceutically acceptable salts thereof are excluded:

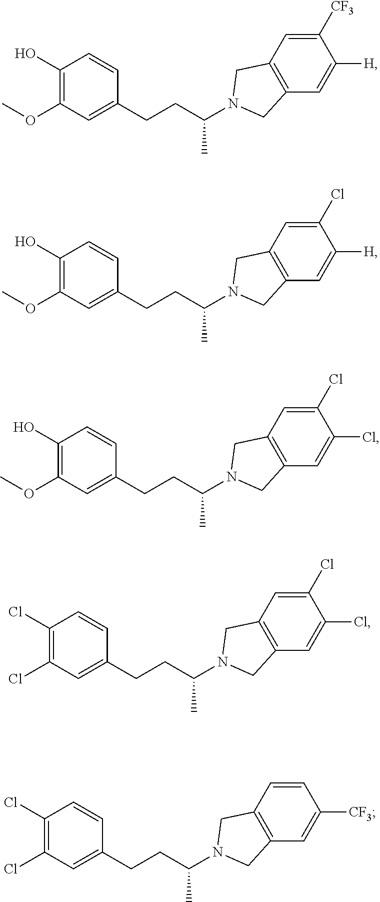

B. a compound of Formula IA

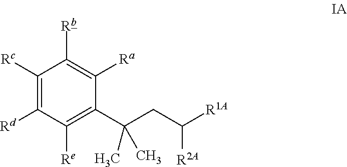

or pharmaceutically acceptable salt thereof:
wherein:
each of R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino;
R^{1A} is selected from the group consisting of hydrogen, alkyl, phenyl, or —CH═C(CH₃)₂; and
R^{2A} is an optionally substituted cyclic amino group.

Some embodiments of the present disclosure are directed to a method of treating dry age-related macular degeneration dry (AMD), comprising administering to a subject in need thereof, a therapeutically effective amount of a compound selected from the group consisting of:

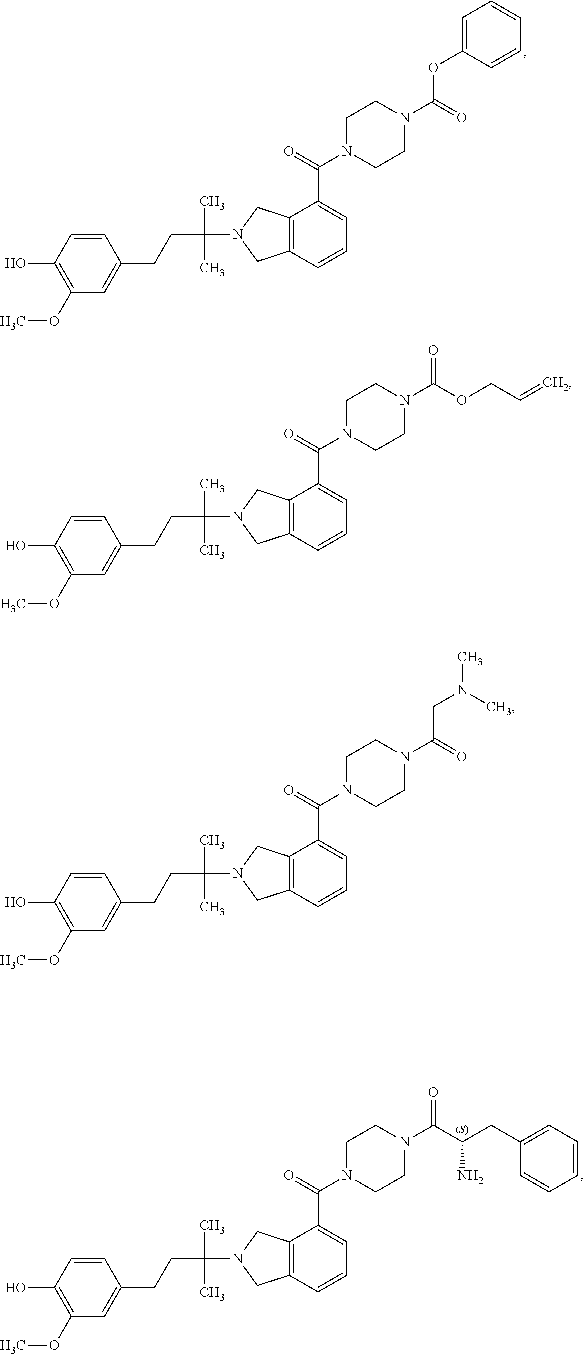

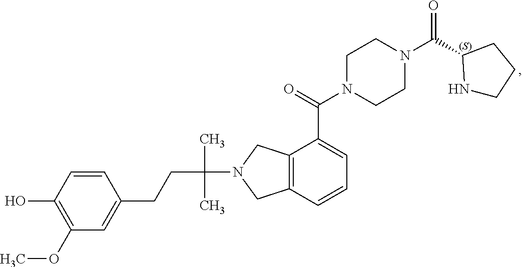

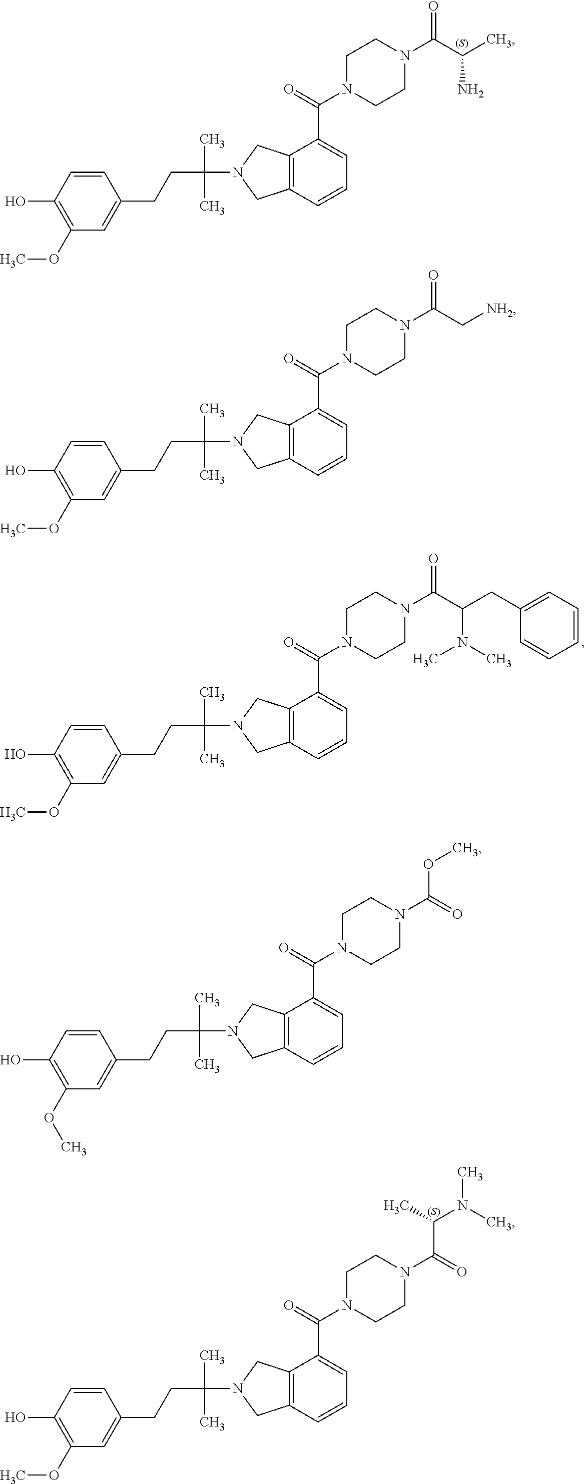

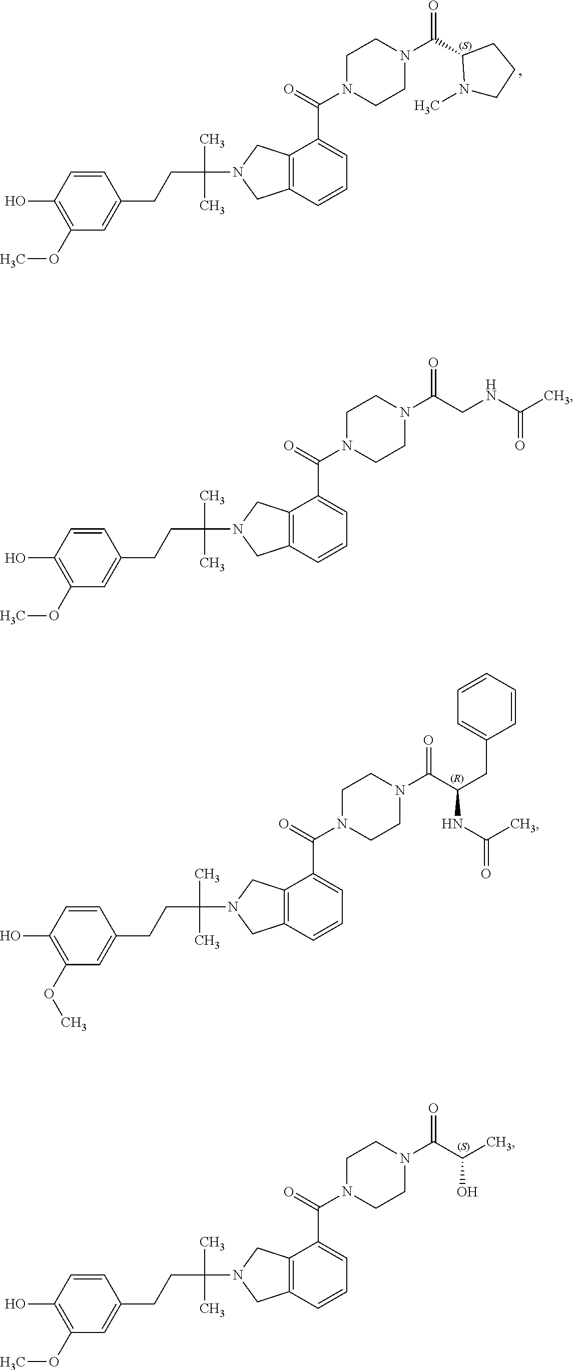

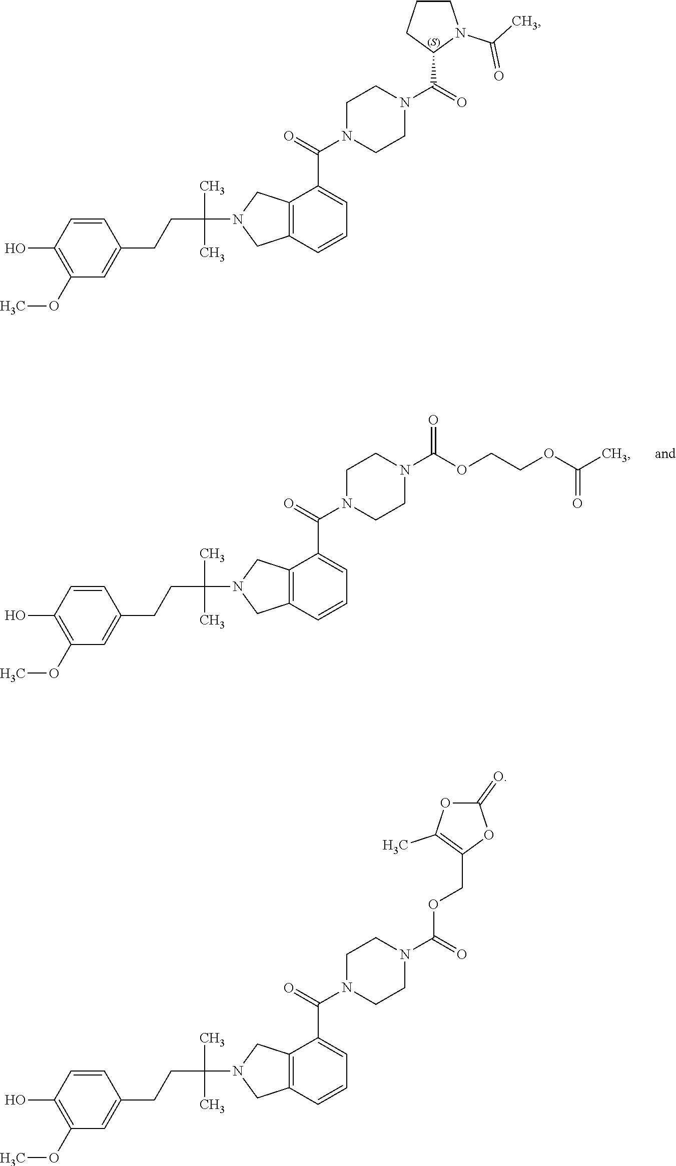

Some embodiments describe the use of a compound selected from

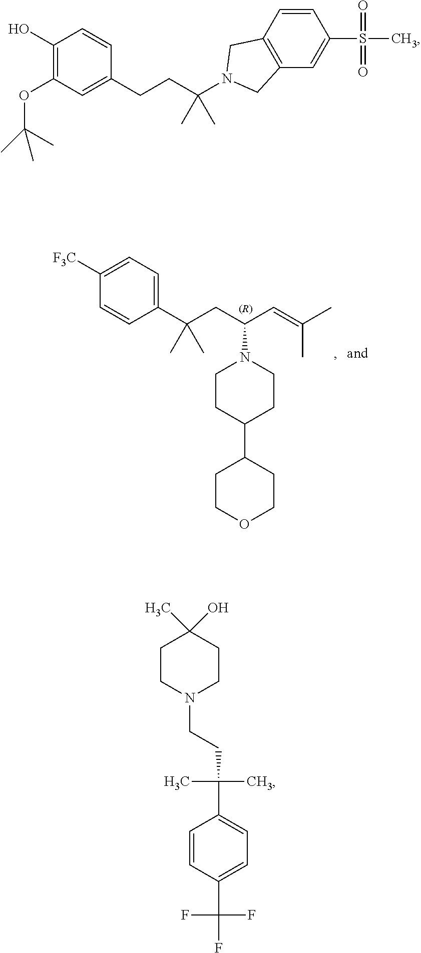

or a pharmaceutically acceptable salt thereof, in the manufacture of a medicament for the treatment of dry age-related macular degeneration.

Some embodiments describe use of a composition comprising a compound elected

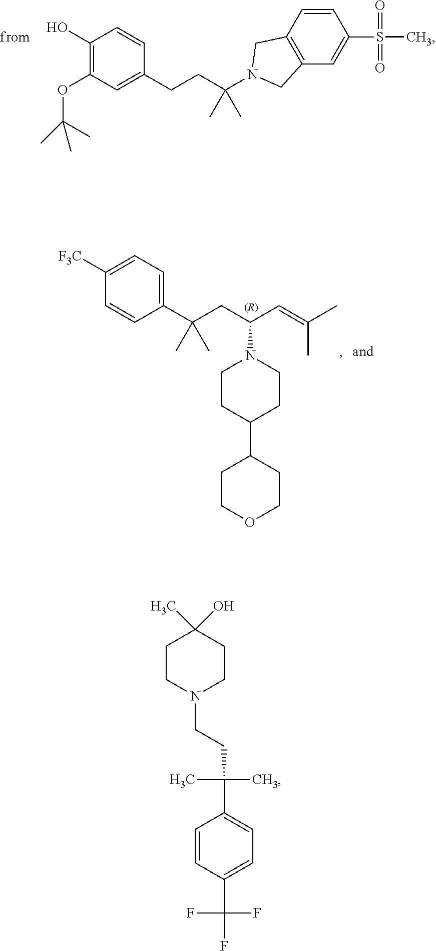

or a pharmaceutically acceptable salt thereof, and a pharmaceutically acceptable excipient; in the manufacture of a medicament for the treatment of dry age-related macular degeneration.

Some embodiments describe a method of treating dry age-related macular degeneration comprising administering to the subject a therapeutically effective amount of a pharmaceutical composition according to any embodiment described herein.

Some embodiments describe use of a compound according to according to any embodiment described herein, in the manufacture of a medicament for the treatment of dry age-related macular degeneration

### BRIEF DESCRIPTION OF THE DRAWINGS

FIGS. 1A and 1B depict cell survival of retinal pigment epithelial cells after treatment with sigma-2 receptor modulators (FIG. 1A=Compound B and Compound C; FIG. 1B=Compound A).

FIGS. 2A and 2B depict photoreceptor outer segment trafficking after treatment with Abeta oligomers in the presence or absence of sigma 2 receptor modulators (FIG. 2A=Compound A and Compound C; FIG. 2B=Compound B).

FIGS. 3A and 3B depict photoreceptor outer segment trafficking after treatment with an oxidative stressor in the presence or absence of sigma 2 receptor modulators (FIG. 3A=Compound A and Compound C; FIG. 3B=Compound B).

FIG. 4 depicts quantification of an autophagy-related protein after oxidative stress by an oxidative stressor in the presence or absence of sigma-2 receptor modulators.

FIG. 5 depicts quantification of the concentration of the sigma-2 receptor modulator Compound A in rat uveal tract/retina and brain.

FIG. 6 depicts quantification of the concentration of the sigma-2 receptor modulator Compound A in mouse plasma, brain, and retina.

FIG. 7 depicts quantification of the concentration of the sigma-2 receptor modulator, Compound B, in mouse plasma, brain and retina.

FIG. 8 depicts quantification of retinal ganglion cell density in an in vivo model of glaucoma in the presence or absence of sigma-2 receptor modulator.

FIGS. 9A and 9B depict quantification of electrical activity in retinal ganglion cells in an in vivo model of glaucoma in the presence or absence of sigma-2 receptor modulator.

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein, are incorporated by reference in their entirety. Nothing herein is to be construed as an admission that the invention is not entitled to antedate such disclosure by virtue of prior invention.

### Definitions

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed.

At various places in the present specification, substituents of compounds of the disclosure are disclosed in groups or in ranges. It is specifically intended that embodiments of the disclosure include each and every individual subcombination of the members of such groups and ranges. For example, the term “C₁₋₆ alkyl” is specifically intended to individually disclose, e.g. methyl (C₁ alkyl), ethyl (C₂ alkyl), propyl (C₃ alkyl), butyl (C₄ alkyl), pentyl (C₅ alkyl), and hexyl (C₆ alkyl) as well as, e.g. C₁-C₂ alkyl, C₁-C₃ alkyl, C₁-C₄ alkyl, C₂-C₃ alkyl, C₂-C₄ alkyl, C₃-C₆ alkyl, C₄-C₅ alkyl, and C₅-C₆ alkyl.

The articles “a” and “an” as used herein, mean “one or more” or “at least one,” unless otherwise indicated. That is, reference to any element of the present invention by the indefinite article “a” or “an” does not exclude the possibility that more than one of the element is present.

As used herein, the term “about” means plus or minus 10% of the numerical value of the number with which it is being used. Therefore, about 50 mL means in the range of 45 mL-55 mL.

“Abeta species” or “Aβ” shall include compositions comprising soluble amyloid peptide-containing components such as Abeta monomers, Abeta oligomers, or complexes of Abeta peptide (in monomeric, dimeric or polymeric form) with other soluble peptides or proteins as well as other soluble Abeta assemblies, including any processed product of amyloid precursor protein. Soluble Aβ oligomers are known to be neurotoxic. Even Aβ 1-42 dimers are known to impair synaptic plasticity in mouse hippocampal slices. In one theory known in the art, native Aβ 1-42 monomers are considered neuroprotective, and self-association of Aβ monomers into soluble Abeta oligomers is required for neurotoxicity. However, certain Aβ mutant monomers (arctic mutation (E22G) are reported to be associated with familial Alzheimer's Disease.

Unless specifically indicated, the term “active ingredient” is to be understood as referring to a compound according to any embodiment describe herein.

“Administering,” or “administration” and the like, when used in conjunction with the compounds of the disclosure refers to providing the compounds or pharmaceutical compositions according to any of the embodiments described herein, to a subject in need of treatment. Preferably the subject is a mammal, more preferably a human. The present invention comprises administering the pharmaceutical composition of the invention alone or in conjunction with another therapeutic agent. When a pharmaceutical composition of the invention is administered in conjunction with another therapeutic agent, the pharmaceutical composition of the invention and the other therapeutic agent. can be administered at the same time or different times.

The term “agonist” refers to a compound, the presence of which results in a biological activity of a receptor that is the same as the biological activity resulting from the presence of a naturally occurring ligand for the receptor.

The term “alkanoyl” or “alkylcarbonyl” as used herein, is meant to refer to an alkyl group attached to a carbonyl radical. An example of an alkanoyl is

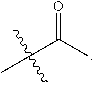 [SMILES: *C(C)=O]

As used herein, the term “alkyl” is meant to refer to a saturated hydrocarbon group which is straight-chained or branched. Example alkyl groups include, but are not limited to, methyl (Me), ethyl (Et), propyl (e.g. n-propyl and isopropyl), butyl (e.g. n-butyl, isobutyl, t-butyl), pentyl (e.g. n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. “C₁-C₁₀ alkyl” or “C₁₋₁₀ alkyl”, is intended to include C₁, C₂, C₃, C₄, C₅, C₆, C₇, C₈, C₉, and C₁₀ alkyl groups. Additionally, for example, “C₁-C₆ alkyl” or “C₁₋₆ alkyl” denotes alkyl having 1 to 6 carbon atoms. The term “alkylene” refers to a divalent alkyl linking group. An example of alkylene is methylene (CH₂).

As used herein, “alkenyl” is intended to include hydrocarbon chains of either straight or branched configuration with one or more, preferably one to three, carbon-carbon double bonds that may occur in any stable point along the chain. For example, “C₂-C₆ alkenyl” or “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, and 4-methyl-3-pentenyl.

The term “alkoxy” or “alkyloxy” refers to an —O-alkyl group. “C₁-C₆ alkoxy” or “C₁₋₆ alkoxy” (or alkyloxy), is intended to include C₁, C₂, C₃, C₄, C₅, and C₆, alkoxy groups. Examples of alkoxy groups include, but are not limited to, methoxy, ethoxy, propoxy (e.g., n-propoxy and isopropoxy), and t-butoxy.

The term “alkoxyxlkoxy” refers to an alkoxy group attached to an alkoxy group. An example of an alkoxy group includes —O—(CH₂)₂—OCH₃.

As used herein, “alkynyl” is intended to include hydrocarbon chains of either straight or branched configuration having one or more, preferably one to three, carbon-carbon triple bonds that may occur in any stable point along the chain. For example, “C₂-C₆ alkynyl” is intended to include C₂, C₃, C₄, C₅, and C₆ alkynyl groups; such as ethynyl, propynyl, butynyl, pentynyl, and hexynyl.

As used herein, an “amyloid beta effect,” for example, a “nonlethal amyloid beta effect,” or “Abeta oligomer effect,” refers to an effect, particularly a nonlethal effect, on a cell that is contacted with an Abeta species. For example, it has been found that when a neuronal cell is contacted with a soluble Amyloid-beta (“Abeta”) oligomer, the oligomers bind to a subset of synapses on a subset of neuronal cells in vitro. This binding can be quantified in an assay measuring Abeta oligomer binding in vitro for example. Another documented effect of Abeta species is a reduction in synapse number, which has been reported to be about 18% in the human hippocampus (Scheff et al, 2007) and can be quantified (for example, in an assay measuring synapse number). As another example, it has been found that, when a neuronal cell is contacted with an Amyloid-beta (“Abeta”) oligomer, membrane trafficking is modulated and alteration of membrane trafficking ensues. This abnormality can be visualized with many assays, including but not limited to, an MTT assay. For example, yellow tetrazolium salts are endocytosed by cells and the salts are reduced to insoluble purple formazan by enzymes located within vesicles in the endosomal pathway. The level of purple formazan is a reflection of the number of actively metabolizing cells in culture, and reduction in the amount of formazan is taken as a measure of cell death or metabolic toxicity in culture. When cells that are contacted with a yellow tetrazolium salt are observed through a microscope, the purple formazan is first visible in intracellular vesicles that fill the cell. Over time, the vesicles are exocytosed and the formazan precipitates as needle-shaped crystals on the outer surface of the plasma membrane as the insoluble formazan is exposed to the aqueous media environment. Still other effects of Abeta species include cognitive decline, such as a decline in the ability to form new memories and memory loss which can be measured in assays using animal models in vivo. In some embodiments, an Abeta effect is selected from Abeta oligomer-induced synaptic dysfunction, for example, as seen in an in vitro assay, such as a membrane trafficking assay, or a synapse loss assay, or Abeta oligomer mediated sigma-2 receptor activation of caspase-3, or Abeta induced neuronal dysfunction, Abeta mediated decrease in long term potentiation (LTP), or in cognitive decline in a behavioral assay, or in a patient in need thereof.

In some embodiments, a test compound is said to be effective to treat cognitive decline or a disease associated therewith when it can inhibit an effect associated with soluble Abeta oligomer species on a neuronal cell more than about 10%, preferably more than 15%, and preferably more than 20% as compared to a negative control. In some embodiments, a test agent is said to be effective when it can inhibit a processed product of amyloid precursor protein-mediated effect more than about 10%, preferably more than 15%, and preferably more than 20% as compared to a positive control. Although the present specification focuses on inhibition of nonlethal effects of Abeta species, such as abnormalities in neuronal metabolism and synapse number reduction, these are shown to correlate with cognitive function and are furthermore expected, over time, to result in reduction (compared to untreated subjects) of downstream measurable symptoms of amyloid pathology, notably clinical symptoms such as 1) fibril or plaque accumulation measured by amyloid imaging agents such as fluorbetapir, PittB or any other imaging agent, 2) synapse loss or cell death as measured by glucose hypometabolism detected with FDG-PET, 3) changes in protein expression or metabolite amount in the brain or body detectable by imaging or protein/metabolite detection in cerebrospinal fluid, brain biopsies or plasma obtained from patients by ELISA, (such as changes in levels and or ratios of Abeta 42, phosphorylated tau, total tau measured by ELISA, or patterns of protein expression changes detectable in an ELISA panel), 4) cerebral vascular abnormalities as measured by the presence of vascular edema or microhemorrhage detectable by MRI and any other symptoms detectable by imaging techniques, and 5) cognitive loss as measured by any administered cognitive test such as ADAS-Cog, MMSE, CBIC or any other cognitive testing instrument.

The term “animal” as used herein, includes, but is not limited to, humans and non-human vertebrates such as wild, experimental, domestic and farm animals and pets.

The term “antagonist” refers to an entity, e.g. a compound, antibody or fragment, the presence of which results in a decrease in the magnitude of a biological activity of a receptor. In certain embodiments, the presence of an antagonist results in complete inhibition of a biological activity of a receptor. As used herein, the term “sigma-2 receptor antagonist” is used to describe a compound that acts as a “functional antagonist” at the sigma-2 receptor in that it blocks Abeta effects, for example, Abeta oligomer-induced synaptic dysfunction, for example, as seen in an in vitro assay, such as a membrane trafficking assay, or a synapse loss assay, or Abeta oligomer mediated sigma-2 receptor activation of caspase-3, or in a behavioral assay, or in a patient in need thereof. The functional antagonist may act directly by inhibiting binding of, for example, an Abeta oligomer to a sigma-2 receptor, or indirectly, by interfering with downstream signaling resultant from Abeta oligomer binding the sigma-2 receptor.

As used herein, “aryl” refers to monocyclic or polycyclic (e.g. having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms. In some embodiments, aryl groups have from 5 to about 10 carbon atoms.

As used herein, “arylalkyl” refers to an aryl group attached to an alkyl radical. In preferred embodiments the alkyl is a C₁₋₆ alkyl.

The term “aroyl” or “arylcarbonyl” as used herein, refers to an aryl group attached to a carbonyl radical. Examples of aroyl include but are not limited to benzoyl.

As used herein the term “brain penetrability” refers to the ability of a drug, antibody or fragment, to cross the blood-brain barrier. In some embodiments, an animal pharmacokinetic (pK) study, for example, a mouse pharmacokinetic/blood-brain barrier study can be used to determine or predict brain penetrability. In some embodiments various concentrations of a compound or pharmaceutical composition according to any embodiment described herein, can be administered, for example at 3, 10 and 30 mg/kg, for example p.o. for 5 days and various pK properties are measured, e.g., in an animal model. In some embodiments, dose related plasma and brain levels are determined. In some embodiments, brain Cmax>100, 300, 600, 1000, 1300, 1600, or 1900 ng/mL. In some embodiments good brain penetrability is defined as a brain/plasma ratio of >0.1, >0.3, >0.5, >0.7, >0.8, >0.9, preferably >1, and more preferably >2, >5, or >10. In other embodiments, good brain penetrability is defined as greater than about 0.1%, 1%, 5%, greater than about 10%, and preferably greater than about 15% of an administered dose crossing the BBB after a predetermined period of time. In certain embodiments, the dose is administered orally (p.o.). In other embodiments, the dose is administered intravenously (i.v.), prior to measuring pK properties.

As used herein, “cognitive decline” can be any negative change in an animal's cognitive function. For example cognitive decline, includes but is not limited to, memory loss (e.g. behavioral memory loss), failure to acquire new memories, confusion, impaired judgment, personality changes, disorientation, or any combination thereof. A compound that is effective to treat cognitive decline can be thus effective by restoring long term neuronal potentiation (LTP) or long term neuronal depression (LTD) or a balance of synaptic plasticity measured electrophysiologically; inhibiting, treating, and/or abatement of neurodegeneration; inhibiting, treating, and/or abatement of general amyloidosis; inhibiting, treating, abatement of one or more of amyloid production, amyloid assembly, amyloid aggregation, and amyloid oligomer binding; inhibiting, treating, and/or abatement of a nonlethal effect of one or more of Abeta species on a neuron cell (such as synapse loss or dysfunction and abnormal membrane trafficking); and any combination thereof. Additionally, that compound can also be effective in treating Abeta related neurodegenerative diseases and disorders including, but not limited to dementia, including but not limited to Alzheimer's Disease (AD) including mild Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD), preclinical Alzheimer's Disease (PCAD); and Cognitive Impairment No Dementia (CIND).

As used herein, the term “contacting” refers to the bringing together or combining of molecules (or of a molecule with a higher order structure such as a cell or cell membrane) such that they are within a distance that allows for intermolecular interactions such as the non-covalent interaction between two peptides or one protein and another protein or other molecule, such as a small molecule. In some embodiments, contacting occurs in a solution in which the combined or contacted molecules are mixed in a common solvent and are allowed to freely associate. In some embodiments, the contacting can occur at or otherwise within a cell or in a cell-free environment. In some embodiments, the cell-free environment is the lysate produced from a cell. In some embodiments, a cell lysate may be a whole-cell lysate, nuclear lysate, cytoplasm lysate, and combinations thereof. In some embodiments, the cell-free lysate is lysate obtained from a nuclear extraction and isolation wherein the nuclei of a cell population are removed from the cells and then lysed. In some embodiments, the nuclei are not lysed, but are still considered to be a cell-free environment. The molecules can be brought together by mixing such as vortexing, shaking, and the like.

The term “cyclic amino” or “cyclic amino group” as used herein, is a heterocycloalkyl or heteroaryl group containing a nitrogen radical, thus allowing bonding through the nitrogen atom. The group can be represented by the formula:

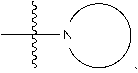 [SMILES: *N]

wherein

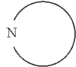

is any heterocyclic or heteroaromatic ring containing 0-3 additional heteroatoms selected from nitrogen, sulfur and oxygen.

The term “cycloalkanoyl” or “cycloalkylcarbonyl” as used herein, is meant to describe a cycloalkyl group attached to a carbonyl radical. Examples of cycloalkanoyl include but are not limited to,

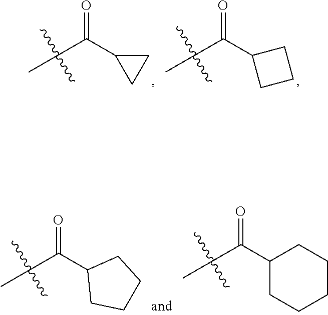

As used herein, “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic (e.g. having 2, 3 or 4 fused rings) ring systems as well as spiro ring systems. A cycloalkyl group can contain from 3 to about 15, from 3 to about 10, from 3 to about 8, from 3 to about 6, from 4 to about 6, from 3 to about 5, or from 5 to about 6 ring-forming carbon atoms. Ring-forming carbon atoms of a cycloalkyl group can be optionally substituted by oxo or sulfido. Example of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e. having a bond in common with) to the cycloalkyl ring, for example, benzo or thienyl derivatives of cyclopentane, cyclopentene, cyclohexane, and the like (e.g. 2,3-dihydro-1H-indene-1-yl, or 1H-inden-2(3H)-one-1-yl). Preferably, “cycloalkyl” refers to cyclized alkyl groups that contain up to 20 ring-forming carbon atoms. Examples of cycloalkyl preferably include cyclopropyl, cyclobutyl, cyclopentyl, cyclohexyl, cycloheptyl, adamantyl, and the like

The term “cycloalkylalkyl” refers to a cycloalkyl group attached to an alkyl radical. In preferred embodiments the alkyl is a C₁₋₆ alkyl.

The term “drug-like properties” is used herein, to describe the pharmacokinetic and stability characteristics of a compound upon administration; including brain penetrability, metabolic stability and/or plasma stability.

As used herein, the term “effective amount” refers to an amount that results in measurable inhibition of at least one symptom or parameter of a specific disorder or pathological process. For example, an amount of a disclosure compound according to any embodiment described herein, that provides a measurably lower synapse reduction in the presence of Abeta oligomer qualifies as an effective amount because it reduces a pathological process even if no clinical symptoms of amyloid pathology are altered, at least immediately.

As used herein, “halo” or “halogen” includes fluorine, chlorine, bromine, and iodine.

As used herein, “haloalkoxy” represents a haloalkyl group as defined herein, with the indicated number of carbon atoms, attached through an oxygen bridge. For example, “C₁-C₆ haloalkoxy” or “C₁₋₆ haloalkoxy”, is intended to include C₁, C₂, C₃, C₄, C₅, and C₆ haloalkoxy groups. An example haloalkoxy group is OCF₃. As used herein, “trihalomethoxy” refers to a methoxy group having three halogen substituents. Examples of trihalomethoxy groups include, but are not limited to, —OCF₃, —OCClF₂, —OCCl₃, and the like.

As used herein, “haloalkyl” is intended to include both branched and straight-chain saturated aliphatic hydrocarbon groups having the specified number of carbon atoms, substituted with one or more halogens. Example haloalkyl groups include, but are not limited to, CF₃, C₂F₅, CHF₂, CCl₃, CHCl₂, C₂Cl₅, CH₂CF₃, and the like.

As used herein, “heteroaryl” groups refer to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms each independently selected from sulfur, oxygen, and nitrogen. Heteroaryl groups include monocyclic and polycyclic (e.g. having 2, 3 or 4 fused rings) systems. Examples of heteroaryl groups include without limitation, pyridyl (a.k.a. pyridinyl), pyrimidinyl, pyrazinyl, pyridazinyl, triazinyl, furyl, quinolyl, isoquinolyl, thienyl, imidazolyl, thiazolyl, indolyl, pyrryl (a.k.a. pyrrolyl), oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 1 to about 5, from about 1 to about 4, from about 1 to about 3, from about 1 to about 2, carbon atoms as ring-forming atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

The term “heterocycloalkoxy” as used herein, refers to an —O-heterocycloalkyl group. An example of a heterocycloalkoxy group is

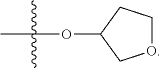 [SMILES: *OC1CCOC1]

As used herein, “heterocycloalkyl” or “heterocyclyl” refers to a non-aromatic heterocyclyl group having up to 20 ring-forming atoms including cyclized alkyl, alkenyl, and alkynyl groups where one or more of the ring-forming carbon atoms is replaced by a heteroatom such as an O, N, or S atom. Heterocycloalkyl groups can be mono or polycyclic (e.g. both fused and spiro systems). For example, “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. Ring-forming carbon atoms and heteroatoms of a heterocycloalkyl group can be optionally substituted by oxo or sulfido. For example, a ring-forming S atom can be substituted by 1 or 2 oxo (i.e. form a S(O) or S(O)₂). For example, a ring-forming C atom can be substituted by oxo (i.e. form carbonyl). Also included in the definition of heterocycloalkyl are moieties that have one or more aromatic rings fused (i.e. having a bond in common with) to the nonaromatic heterocyclic ring, for example pyridinyl, thiophenyl, phthalimidyl, naphthalimidyl, and benzo derivatives of heterocycles such as indoline, isoindoline, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido. In some embodiments, the heterocycloalkyl group has from 2 to about 20 carbon atoms or 3 to 20 carbon atoms. In some embodiments, the heterocycloalkyl group contains 3 to about 14, 3 to about 7, or 5 to 6 ring-forming atoms. In some embodiments, the heterocycloalkyl group has 1 to 4 heteroatoms. In some embodiments, the heterocycloalkyl group contains 0 to 3 double bonds. In some embodiments, the heterocycloalkyl group contains 0 to 2 triple bonds.

In the present application, the term “high affinity” is intended to mean a compound which exhibits a Kᵢ value of less than 600 nM, 500 nM, 400 nM, 300 nM, 200 nM, less than 150 nM, less than 100 nM, less than 80 nM, less than 60 nM, or preferably less than 50 nM in a sigma receptor binding assay, for example against [3H]-DTG, as disclosed by Weber et al., Proc. Natl. Acad. Sci (USA) 83: 8784-8788 (1986), incorporated herein by reference, which measures the binding affinity of compounds toward both the sigma-1 and sigma-2 receptor sites. Especially preferred compounds exhibit Kᵢ values of less than about 150 nM, preferably less than 100 nM, less than about 60 nM, less than about 10 nM, or less than about 1 nM against [3H]-DTG.

The terms “hydroxyl” and “hydroxy” are used interchangeably to mean an OH group.

The term “improves” is used to convey that the disclosure changes either the characteristics and/or the physical attributes of the tissue to which it is being provided, applied or administered. The term “improves” may also be used in conjunction with a disease state such that when a disease state is “improved” the symptoms or physical characteristics associated with the disease state. are diminished, reduced, eliminated, delayed or averted.

The term “inhibiting” includes the blockade, aversion of a certain result or process, or the restoration of the converse result or process. In terms of prophylaxis or treatment by administration of a compound of the disclosure, “inhibiting” includes protecting against (partially or wholly) or delaying the onset of symptoms, alleviating symptoms, or protecting against, diminishing or eliminating a disease, condition or disorder.

The term “inhibiting trafficking deficits” refers to the ability to block soluble Aβ oligomer-induced membrane trafficking deficits in a cell, preferably a neuronal cell. A compound capable of inhibiting trafficking deficits has an EC₅₀<20 PM, less than 15 μM, less than 10 μM, less than 5 μM, and preferably less than 1 μM in the membrane trafficking assay, and further is capable of at least 50%, preferably at least 60%, and more preferably at least 70% maximum inhibition of the Abeta oligomer effects of soluble Abeta oligomer-induced membrane trafficking deficits, for example, as described in Example 6.

The term “log P” refers to the partition coefficient of a compound. The partition coefficient is the ratio of concentrations of un-ionized compound in each of two solution phases, for example, octanol and water. To measure the partition coefficient of ionizable solute compounds, the pH of the aqueous phase is adjusted such that the predominant form of the compound is un-ionized. The logarithm of the ratio of concentrations of the un-ionized solute compound in the solvents is called log P. The log P is a measure of lipophilicity. For example, log P_{oct/wat}=log ([solute]_{octanol}/[solute]_{un-ionized, water}).

As used herein the term “metabolic stability” refers to the ability of a compound to survive first-pass metabolism (intestinal and hepatic degradation or conjugation of a drug administered orally). This can be assessed, for example, in vitro by exposure of the compounds to mouse or human hepatic microsomes. In some embodiments, good metabolic stability refers to a t_{1/2}>5 min, >10 min, >15 minutes, >20 minutes, and preferably >30 min upon exposure of a compound to mouse or human hepatic microsomes. In some embodiments, good metabolic stability refers to an Intrinsic Clearance Rate (Clᵢₙₜ) of <300 uL/min/mg, preferably ≤200 uL/min/mg, and more preferably ≤100 uL/min/mg.

The term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl group.

As used herein, the term “natural ligand” refers to a ligand present in a subject that can bind to a protein, receptor, membrane lipid or other binding partner in vivo or that is replicated in vitro. The natural ligand can be synthetic in origin, but must also be present naturally and without human intervention in the subject. For example, Abeta oligomers are known to exist in human subjects. Therefore the Abeta oligomers found in a subject would be considered natural ligands. The binding of Abeta oligomers to a binding partner can be replicated in vitro using recombinant or synthetic techniques, but the Abeta oligomer would still be considered a natural ligand regardless of how the Abeta oligomer is prepared or manufactured. A synthetic small molecule that can also bind to the same binding partner is not a natural ligand if it does not exist in a subject. For example, compounds which are described herein, are not normally present in a subject, and, therefore, would not be considered natural ligands.

As used herein, the term “a neuronal cell” can be used to refer to a single cell or to a population of cells. In some embodiments, the neuronal cell is a primary neuronal cell. In some embodiments, the neuronal cell is an immortalized or transformed neuronal cell or a stem cell. A primary neuronal cell is a neuronal cell that cannot differentiate into other types of neuronal cells, such as glia cells. A stem cell is one that can differentiate into neurons and other types of neuronal cells such as glia. In some embodiments, assays utilize a composition comprising at least one neuronal cell is free of glia cells. In some embodiments, the composition comprises less than about 30%, 25%, 20%, 15%, 10%, 5%, or 1% of glia cells, which are known to internalize and accumulate Abeta. The primary neuronal cell can be derived from any area of the brain of an animal. In some embodiments, the neuronal cell is a hippocampal or cortical cell. The presence of glia cells can be determined by any method. In some embodiments, glia cells are detected by the presence of GFAP and neurons can be detected by staining positively with antibodies directed against MAP2.

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valence of the designated atom or moiety is not exceeded, and that the substitution results in a stable compound. For example, if a methyl group (i.e. CH₃) is optionally substituted, then up to 3 hydrogen atoms on the carbon atom can be replaced with substituent groups. Substituent groups include, but are not limited to, alkanoyl, alkoxy, alkoxyalkyl, (alkoxy)alkoxyalkyl, alkoxycarbonyl, alkyl, aryloxy, aryloyl, cycloalkanoyl, substituted or unsubstituted C₃-C₁₀ cycloalkyl, —OC(O)NCH(CH₃)₂, (N,N-dimethylamino)pyridinyl, (N,N-dimethylamino)sulfonyl, halo, heterocyclyl, (heterocyclyl)alkoxyalkyl, heterocycloalkyl, hydroxyl, hydroxyalkyl, methylpiperidinyl, methylsulfonyl, methylsulfonylphenyl, morpholinylpyridinyl, optionally substituted C₁-C₁₀ alkyl, optionally substituted C₅-C₁₀ aryl, optionally substituted C₃-C₁₀ heteroaryl, perfluoroalkyl, phenyl, piperidinyl, pyrrolidinylpyridinyl, tetrahydropyranyl, CF₃. A substituted alkyl group for example indicates that one or more hydrogen atoms on the alkyl group is replaced with a substituent group, selected from but not limited to, halo, hydroxyl, alkoxy, heterocycloalkoxy, alkoxyalkoxy, C(O)OMe, and C(O)OEt. A substituted aryl group for example, indicates that one or more hydrogen atoms on the aryl group is replaced with a substituent group, selected from but not limited to, —SO₂Me or phenyl group. A substituted heteroaryl group for example, indicates that one or more hydrogen atoms on the heteroaryl group is replaced with a substituent group, selected from, but not limited to, heterocycloalkyl, heteroaryl, N,N-dimethylamino. A substituted heterocycloalkyl group for example, indicates that one or more hydrogen atoms on the heterocycloalkyl group is replaced with a substituent group, selected from, but not limited to, heterocyclalkyl, heteroaryl, N,N-dimethylamino, hydroxyl, alkoxy, alkoxycarbonyl, alkyl, aryl, sulfonyl, dimethylaminosulfonyl, aroyl, cycloalkanoyl, alkanoyl and —OC(O)NCH(CH₃)₂. In some instances two hydrogen atoms on the same carbon of, for example, a heterocyclyl or alkyl group are replaced with a group to form a spiro compound selected from but not limited to, for example,

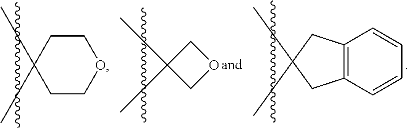

The term “partial agonist” refers to a compound the presence of which results in a biological activity of a receptor that is of the same type as that resulting from the presence of a naturally occurring ligand for the receptor, but of a lower magnitude.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “selectivity” or “selective” refers to a difference in the binding affinity of a compound (Kᵢ) for a sigma receptor, for example, a sigma-2 receptor, compared to a non-sigma receptor. The compound possess high selectivity for a sigma receptor in synaptic neurons. The Kᵢ for a sigma-2 receptor or both a sigma-2 and a sigma-1 receptor is compared to the K; for a non-sigma receptor. In some embodiments, the compound is a selective sigma-2 receptor antagonist, or sigma-1 receptor ligand, and has at least 10-fold, 20-fold, 30-fold, 50-fold, 70-fold, 100-fold, or 500-fold higher affinity, or more, for binding to a sigma receptor compared to a non-sigma receptor as assessed by a comparison of binding dissociation constant Kᵢ values, or IC₅₀ values, or binding constant, at different receptors. Any known assay protocol can be used to assess the Kᵢ or IC₅₀ values at different receptors, for example, by monitoring the competitive displacement from receptors of a radiolabeled compound with a known dissociation constant, for example, by the method of Cheng and Prusoff (1973) (Biochem. Pharmacol. 22, 3099-3108), or specifically as provided herein.

As used herein the term “plasma stability” refers to the degradation of compounds in plasma, for example, by enzymes such as hydrolases and esterases. Any of a variety of in vitro assays can be employed. Test compounds are incubated in plasma over various time periods. The percent parent compound (analyte) remaining at each time point reflects plasma stability. Poor stability characteristics can tend to have low bioavailability. Good plasma stability can be defined as greater than 50% analyte remaining after 30 min, greater than 50% analyte remaining after 45 minutes, and preferably greater than 50% analyte remaining after 60 minutes.

“Sigma-2 ligand” refers to a compound that binds to a sigma-2 receptor and includes agonists, antagonists, partial agonists, inverse agonists and simply competitors for other ligands of this receptor or protein.

The term “sigma-2 receptor antagonist compound” refers to a compound that binds to a sigma-2 receptor in a measurable amount and acts as a functional antagonist with respect to Abeta effects oligomer induced synaptic dysfunction resultant from sigma-2 receptor binding.

The terms “subject,” “individual” or “patient” are used interchangeably and as used herein, are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients. The methods are particularly suitable for treating human patients having a disease or disorder described herein.

A “test compound” is a compound according to any embodiment described herein that is being tested in any test. Tests include any in vivo or in vitro test, computer model or simulation, virtual drug trial, stem cell and genetic testing methods, non-invasive imaging techniques and the like.

As used herein, the term “therapeutic” means an agent utilized to treat, combat, ameliorate, protect against or improve an unwanted condition or disease of a subject.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). A therapeutically effective amount of a compound, according to any embodiment described herein, may broadly range from 0.01 mg/kg to about 500 mg/kg, about 0.01 to about 250 mg/kg, about 0.01 to about 25 mg/kg, about 0.05 mg/kg to about 20 mg/kg, about 0.1 mg/kg to about 400 mg/kg, about 0.1 mg/kg to about 200 mg/kg, about 0.1 mg/kg to about 25 mg/kg, about 0.1 to about 10 mg/kg, about 0.2 to about 5 mg/kg, about 1 mg/kg to about 300 mg/kg, about 10 mg/kg to about 100 mg/kg, body weight. The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue. The total daily dose of the compounds according to any embodiment described herein administered to a human or other animal in single or in divided doses can be in amounts, for example, from about 0.01 mg/kg to about 500 mg/kg, about 0.01 to about 250 mg/kg, about 0.01 to about 25 mg/kg, about 0.05 mg/kg to about 20 mg/kg, about 0.1 mg/kg to about 400 mg/kg, about 0.1 mg/kg to about 200 mg/kg, about 0.1 mg/kg to about 25 mg/kg, about 0.1 to about 10 mg/kg, about 0.2 to about 5 mg/kg, about 1 mg/kg to about 300 mg/kg, about 10 mg/kg to about 100 mg/kg, body weight per day. Single dose pharmaceutical compositions of any embodiment described herein, may contain such amounts or submultiples thereof to make up the daily dose. For example, the compounds according to any embodiment described herein, may be administered on a regimen of 1 to 4 times per day, such as once, twice, three times or four times per day. In some embodiments, the therapeutically effective amount of a compound according to any embodiment disclosed herein, can range between about 0.01 and about 25 mg/kg/day. In some embodiments the therapeutically effective amount is between a lower limit of about 0.01 mg/kg of body weight, about 0.1 mg/kg of body weight, about 0.2 mg/kg of body weight, about 0.3 mg/kg of body weight, about 0.4 mg/kg of body weight, about 0.5 mg/kg of body weight, about 0.60 mg/kg of body weight, about 0.70 mg/kg of body weight, about 0.80 mg/kg of body weight, about 0.90 mg/kg of body weight, about 1 mg/kg of body weight, about 2.5 mg/kg of body weight, about 5 mg/kg of body weight, about 7.5 mg/kg of body weight, about 10 mg/kg of body weight, about 12.5 mg/kg of body weight, about 15 mg/kg of body weight, about 17.5 mg/kg of body weight, about 20 mg/kg of body weight, about 22.5 mg/kg of body weight, and about 25 mg/kg of body weight; and an upper limit of 25 mg/kg of body weight, about 22.5 mg/kg of body weight, about 20 mg/kg of body weight, about 17.5 mg/kg of body weight, about 15 mg/kg of body weight, about 12.5 mg/kg of body weight, about 10 mg/kg of body weight, about 7.5 mg/kg of body weight, about 5 mg/kg of body weight, about 2.5 mg/kg of body weight, about 1 mg/kg of body weight, about 0.9 mg/kg of body weight, about 0.8 mg/kg of body weight, about 0.7 mg/kg of body weight, about 0.6 mg/kg of body weight, about 0.5 mg/kg of body weight, about 0.4 mg/kg of body weight, about 0.3 mg/kg of body weight, about 0.2 mg/kg of body weight, about 0.1 mg/kg of body weight, and about 0.01 mg/kg of body weight. In some embodiments, the therapeutically effective amount is about 0.1 mg/kg/day to about 10 mg/kg/day; in some embodiments the therapeutically effective amount is about 0.2 and about 5 mg/kg/day. In some embodiments, treatment regimens according to the disclosure comprise administration to a patient in need of such treatment will usually include from about 1 mg to about 5000 mg, about 10 mg to about 2000 mg, about 10 mg to about 200 mg, about 20 to about 1000 mg, about 20 to about 500 mg, about 20 to about 400 mg, about 40 to about 800 mg, about 50 mg to about 500 mg, about 80 to about 1600 mg and about 50 mg, of a compound according to any embodiment disclosed herein, or a pharmaceutically acceptable salt thereof, per day in single or multiple doses. In some embodiments the therapeutically effective amount is a total daily dose of 50 mg to 500 mg. In some embodiments, the daily dose is between a lower limit of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg; about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg; about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, 300 mg, about 305 mg, about 310 mg, about 315 mg; about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395, about 400 mg, about 405 mg, about 410 mg, about 415 mg; about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, and about 500 mg and an upper limit of about 500 mg, about 495 mg, about 490 mg, about 485 mg, about 480 mg, about 475 mg, about 470 mg, about 465 mg, about 460 mg, about 455 mg, about 450 mg, about 445 mg, about 440 mg, about 435 mg, about 430 mg, about 425 mg, about 420 mg, about 415 mg, about 410 mg, about 405 mg, about 400 mg, about 395 mg, about 390 mg, about 385 mg, about 380 mg, about 375 mg, about 370 mg, about 365 mg, about 360 mg, about 355 mg, about 350 mg, about 345 mg, about 340 mg, about 335 mg, about 330 mg, about 325 mg, about 320 mg, about 315 mg, about 310 mg, about 305 mg about 300 mg, about 295 mg, about 290 mg, about 285 mg, about 280 mg, about 275 mg, about 270 mg, about 265 mg, about 260 mg, about 255 mg, about 250 mg, about 245 mg, about 240 mg, about 235 mg, about 230 mg, about 225 mg, about 220 mg, about 215 mg, about 210 mg, about 205 mg 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 95 mg, about 90 mg; about 85 mg, about 80 mg, about 75 mg, about 70 mg, about 65 mg, about 60 mg, about 55 mg, and about 50 mg of a compound according to any embodiment herein. In some embodiments, the total daily dose is about 50 mg to 150 mg. In some embodiments, the total daily dose is about 50 mg to 250 mg. In some embodiments, the total daily dose is about 50 mg to 350 mg. In some embodiments, the total daily dose is about 50 mg to 450 mg. In some embodiments, the total daily dose is about 50 mg. It will be understood that the pharmaceutical formulations of the disclosure need not necessarily contain the entire amount of the compound that is effective in treating the disorder, as such effective amounts can be reached by administration of a plurality of divided doses of such pharmaceutical formulations. The compounds may be administered on a regimen of 1 to 4 times per day, such as once, twice, three times or four times per day.

The term “therapeutic phenotype” is used to describe a pattern of activity for compounds in the in vitro assays that is predictive of behavioral efficacy. A compound that (1) selectively binds with high affinity to a sigma-2 receptor, and (2) acts as a functional antagonist with respect to Abeta oligomer-induced effects in a neuron, is said to have the “therapeutic phenotype” if (i) it blocks or reduces Aβ-induced membrane trafficking deficits; (ii) it blocks or reduces Aβ-induced synapse loss and (iii) it does not affect trafficking or synapse number in the absence of Abeta oligomer. This pattern of activity in the in vitro assays is termed the “therapeutic phenotype” and is predictive of behavioral efficacy.

The term “therapeutic profile” is used to describe a compound that meets the therapeutic phenotype, and also has good brain penetrability (the ability to cross the blood brain barrier), good plasma stability and good metabolic stability.

The term “tissue” refers to any aggregation of similarly specialized cells which are united in the performance of a particular function.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

### Human Amyloid Beta and Sigma-2 Antagonists

Dysfunction of retinal pigment epithelial (RPE) cells play a key role in the development of geographic atrophic (GA) dry age-related macular degeneration (AMD). There is strong evidence to indicate that the Alzheimer's disease-linked amyloid beta (Aβ) proteins. Smaller, soluble Aβ oligomers interfere with a number of signaling pathways.

A subset of sigma-2 receptor binding sites/signaling pathways are relevant to Aβ oligomer signaling in Alzheimer's Disease and knockdown of sigma-2 receptors protects retinal pigment epithelial cells (RPE) from oxidative stress-induced cell death. Sigma-2 receptors are implicated in many signaling pathways such as heme binding, Cytochrome P450 metabolism, cholesterol synthesis, progesterone signaling, apoptosis and membrane trafficking. Dysfunction of retinal pigment epithelial (RPE) cells play a key role in the development of geographic atrophic (GA) dry age-related macular degeneration (AMD) and there is strong evidence to indicate that the Alzheimer's disease-linked amyloid beta (Aβ) proteins are involved in this process.

Vision loss is becoming a major public health problem as the population ages. The American Foundation for the Blind reports that blindness or low vision affects 32.2 million Americans of age 18 and over. The most common eye diseases in Americans age 40 and over are age-related macular degeneration, glaucoma, cataracts and diabetic retinopathy. The causes for these diseases are varied, and include injury, exposure to 15 toxins, underlying health conditions (e.g., diabetes, arteriosclerosis), and genetic factors (e.g., overproduction of aqueous humor). With the exception of cataracts, where the lens can be removed and replaced, there is no cure for these diseases and vision loss is generally permanent.

There is a need for protective compounds that inhibit, reduce, or otherwise treat vision impairment or progression. These protective compounds would be useful in the context of injuries arising from impact or toxic chemicals including counteracting toxic side-effects associated with certain chemotherapeutic regimes, or improving quality of life in populations experiencing progressive vision impairment.

While not being bound by theory, it is proposed that the sigma-2 receptor is a receptor for Abeta oligomer in the retinal pigment epithelial cells (RPE) and the retinal ganglion cells (RGC) of the eye. Various receptors have been proposed in the literature for soluble Abeta oligomers including prion protein, insulin receptor, beta adrenergic receptor and RAGE (receptor for advanced glycation end products). Laurén, J. et al, 2009, Nature, 457(7233): 1128-1132; Townsend, M. et al, J. Biol. Chem. 2007, 282:33305-33312; Sturchler, E. et al, 2008, J. Neurosci. 28(20):5149-5158. Indeed many investigators believe that Abeta oligomer may bind to more than one receptor protein. Without being bound by theory, the present inventors postulate an additional receptor for Abeta oligomer located in the eye.

Without being bound by theory, Abeta oligomers are sigma receptor agonists that bind to sigma protein complexes and cause aberrant trafficking and cell damage. It is demonstrated herein, that compounds described herein that antagonize this interaction and/or sigma receptor function in RPEs and/or RGCs will compete or otherwise interfere with Abeta oligomers to prevent further cell damange and cell death. Such compounds are considered functional sigma-2 receptor antagonists for the treatment of ocular-related neurodegenerative diseases such as age-related macular degeneration.

In some embodiments, a compound of any embodiment described herein, may act as a functional antagonist in a RPE or RGC with respect to inhibiting soluble AP oligomer induced cell damage or cell death, and inhibiting soluble AP oligomer induced deficits in membrane trafficking assays and cell health assays.

In some embodiments, a compound according to any embodiment described herein, that acts as functional antagonist meeting certain in vitro assay criteria detailed herein, will exhibit behavioral efficacy, or be predicted to have behavioral efficacy, in one or more relevant in vitro assays or animal behavioral models.

In accordance with the in vitro assay platform, a compounds of any embodiment described herein, may bind with high affinity to a sigma-2 receptor; acts as a functional antagonist with respect to Abeta oligomer-induced effects in a RPE and RGC; inhibits Abeta oligomer-induced cell damage or death in a RPE or RGC; and does not affect membrane trafficking or cell health in the absence of Abeta oligomer. This pattern of activity in the in vitro assays is termed the “therapeutic phenotype”. The ability of a compound according to any embodiment described herein, to block Abeta oligomer effects in RPEs and RGCs without affecting normal function in the absence of Abeta oligomers meets the criteria for the therapeutic phenotype. A compounds of any embodiment described herein, having a therapeutic phenotype, can block Abeta oligomer-induced RPE and RGC cell damage or death.

In some embodiments, a compound according to any embodiment described herein, exhibits sigma-2 antagonist activity, high affinity for the sigma-2 receptor, and the ability to block soluble Abeta oligomer binding or Abeta oligomer-induced RPE and RGC cell damage or death.

In some embodiments, a compound according to any embodiment described herein, blocks binding between soluble Abeta oligomers and a sigma-2 receptor.

In some embodiments, a compound according to any embodiment described herein, exhibits high affinity for the sigma-2 receptor.

### Methods of Use

Various embodiments are directed to a method of treating an ocular-related neurodegenerative disease, comprising administering to a subject in need thereof, a therapeutically effective amount of a compound as described herein.

Some embodiments are directed to a use of a compound selected from

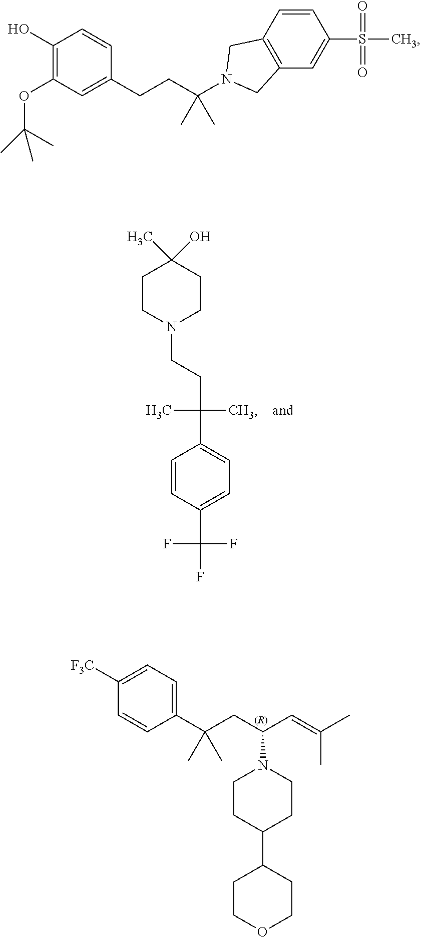

a pharmaceutically acceptable salt thereof, in the manufacture of a medicament for the treatment of dry age-related macular degeneration as described herein.

Some embodiments are directed to a use of a composition comprising a compound

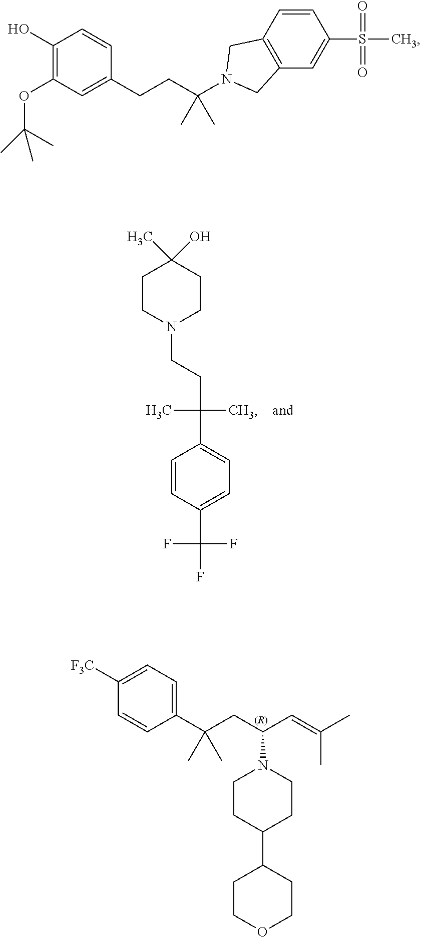

or a pharmaceutically acceptable salt thereof, and a pharmaceutically acceptable excipient; in the manufacture of a medicament for the treatment of dry age-related macular degeneration as described herein.

In some embodiments the ocular-related neurodegenerative disease is selected from the group consisting of glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis.

Various embodiments are directed to a method of treating age-related macular degeneration (AMD), comprising administering to a subject in need thereof, a therapeutically effective amount of a compound described herein.

Some embodiments are directed to a method of treating wet age-related macular degeneration (wet AMD), comprising administering to a subject in need thereof, a therapeutically effective amount of a compound as described herein.

Some embodiments are directed to a method of treating dry age-related macular degeneration (dry AMD), comprising administering to a subject in need thereof, a therapeutically effective amount of a compound as described herein.

Some embodiments are directed to a method of treating geographic atrophic (GA) dry age-related macular degeneration (AMD), comprising administering to a subject in need thereof, a therapeutically effective amount of a compound as described herein.

Some embodiments are directed to a method of preventing cell death in a neuronal cell, comprising administering to a subject in need thereof, a therapeutically effective amount of a compound as described herein.

Some embodiments are directed to a method of preventing cell death in a retinal pigment epithelial cell (RPE), comprising administering to a subject in need thereof, a therapeutically effective amount of a compound as described herein.

Some embodiments are directed to a method of preventing cell death in a retinal ganglion cell (RGC), comprising administering to a subject in need thereof, a therapeutically effective amount of a compound as described herein.

In some embodiments, a compound according to any embodiment described herein, may be protective against cellular dysfunction in retinal pigment epithelial (RPE) cells and retinal ganglion cells (RGCs) associated with dry AMD. In some embodiments, a compound according to any embodiment described herein, may prevent cellular dysfunction associated with dry AMD. In some embodiments, a compound according to any embodiment described herein, may prevent cellular dysfunction associated with dry AMD. wherein the cellular dysfunction may be caused by exposure to inflammatory stimuli, oligomeric Abeta, 4-Hydroxynonenal, or 4-hydroxy-2-nonena (4-HNE), hydrogen peroxide, oxidative stress, and activities of complement C3.

Some embodiments are directed to a method of treating or preventing oxidative stress in RPEs and RGCs, comprising administering to a subject in need thereof, a therapeutically effective amount of a compound as described herein.

In some embodiments, the oxidative stress in RPEs and RGCs results in cellular damage. In some embodiments cellular damage is selected from the group consisting of cytotoxicity, lipid peroxidation, carbonyl formation, formation of reactive oxygen species, changes in mitochondrial membrane potential, changes in mitochondrial mass, changes in mitochondrial function, changes in autophagic flux, loss of lysosomal integrity, changes in lysosomal activity, defects in photoreceptor outer segment (POS) trafficking, accumulation of toxic macromolecules, axonal injury, cell senescence, apoptosis, and cell death. In some embodiments, the method of treating or preventing oxidative stress comprising administering to a subject in need thereof, a therapeutically effective amount of a pharmaceutical composition comprising any compound described herein.

Some embodiments are directed to a method of treating or preventing cytotoxicity in RPEs and RGCs, comprising administering to a subject in need thereof, a therapeutically effective amount of any compound as described herein.

Some embodiments are directed to a method of treating or preventing changes in lysosomal activity in RPEs and RGCs, comprising administering to a subject in need thereof, a therapeutically effective amount of any compound as described herein.

Some embodiments are directed to a method of treating or preventing changes in autophagic flux in RPEs and RGCs, comprising administering to a subject in need thereof, a therapeutically effective amount of any compound as described herein.

Some embodiments are directed to a method of treating or preventing defects in photoreceptor outer segment (POS) trafficking in RPEs, comprising administering to a subject in need thereof, a therapeutically effective amount of any compound as described herein.

Some embodiments are directed to a method of preventing cell death in RPEs and RGCs, comprising administering to a subject in need thereof, a therapeutically effective amount of any compound as described herein.

Some embodiments are directed to a method of preventing apoptosis in RPEs and RGCs, comprising administering to a subject in need thereof, a therapeutically effective amount of any compound as described herein.

Some embodiments are directed to a method of treating or preventing complement C3 dysfunction in RPEs and RGCs, comprising administering to a subject in need thereof, a therapeutically effective amount of any compound as described herein.

In some embodiments, the complement C3 dysfunction in RPEs and RGCs results in cellular damage. In some embodiments cellular damage is selected from the group consisting of cell death, deficits in trans-epithelial electrical resistance (TEER), and deficits in RPE barriers.

Some embodiments are directed to a method of treating or preventing inflammation in RPEs and RGCs, comprising administering to a subject in need thereof, a therapeutically effective amount of an compound as described herein.

In some embodiments, the inflammation is caused by inflammatory stimuli. In some embodiments, inflammatory stimuli are selected from the group consisting of tumor necrosis factor-alpha (TNF-alpha), interferon-gamma (IFNg), 4-Hydroxynonenal, or 4-hydroxy-2-nonena, rotenone, ter-butyl hydroperoxide, and hydrogen peroxide. In some embodiments, the method of treating or preventing inflammation caused by any inflammatory stimuli disclosed herein.

Some embodiments are directed to a methods of slowing the progression of dry age-related macular degeneration (dry AMD), comprising administering to a subject in need thereof, a therapeutically effective amount of any compound as described herein.

Some embodiments are directed to a methods of preventing dry age-related macular degeneration (dry AMD), comprising administering to a subject in need thereof, a therapeutically effective amount of any compound as described herein.

Some embodiments are directed to a methods of slowing the progression of a symptom associated with dry age-related macular degeneration (dry AMD), comprising administering to a subject in need thereof, a therapeutically effective amount of any compound as described herein.

In some embodiments, the symptom associated with dry age-related macular degeneration (dry AMD) is selected from the group consisting of the number of drusen, drusen size, intraocular hypertension, and vision loss. In some embodiments, the method of treating or preventing a symptom associated with dry AMD as described herein, comprising administering to a subject in need thereof, a therapeutically effective amount of a pharmaceutical composition comprising any compound described herein.

### Compounds for Use in the Invention

In some embodiments the compound for use in the invention is a compound selected from the group consisting of:
A. a compound of Formula I,

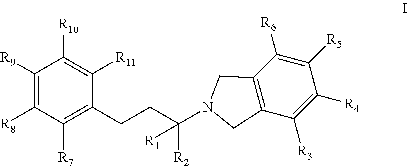

or a pharmaceutically-acceptable salt thereof:
wherein:
each of R₁ and R₂ is independently selected from H, C₁-C₆ alkyl, or CH₂OR′; wherein each R′ if present in R₁, and R₂ is independently H or C₁-C₆ alkyl;
each of R₃, R₄, R₅, and R₆ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁-C₆ alkoxy C₁-C₆alkyl, aryl, heteroaryl, C₃-C₇ cycloalkyl, heterocycloalkyl, alkylaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₃, R₄, R₅, and R₆ is independently selected from the group consisting of H, CH₃, CH₂CH₃, C₃-C₆ alkyl, C₁-C₆ haloalkyl, or optionally substituted aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂, wherein the optionally substituted group is selected from C₁-C₆ alkyl or C₂-C₇ acyl;
or R₃ and R₄, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₃ and R₄ are linked together to form a —O—C₁-C₂ methylene-O— group;
or R₄ and R₅, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁. C₆ alkyl, C₁-C₆haloalkyl, C₁-C₆ alkoxy, C₁-C₆haloalkoxy, aryl, arylalkyl, heteroaryl, heteroarylalkyl, cycloalkyl, and heterocycloalkyl or R₄ and R₅ are linked together to form a —O—C₁₋₂ methylene-O— group;
each of R₇, R₈, R₉, R₁₀, and R₁₁ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁-C₆ alkoxy C₁-C₆ alkyl, aryl, heteroaryl, C₃-C₇ cycloalkyl, heterocycloalkyl, alkylaryl, heteroaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₇, R₈, R₉, R₁₀, and R₁₁ is independently selected from the group consisting of H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂;
or R₇ and R₈, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₇ and R₈ are linked together to form a —O—C₁₋₂ methylene-O— group;
or R₈ and R₉, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₈ and R₉ are linked together to form a —O—C₁₋₂ methylene-O— group;
each n is independently 0, 1, or 2;
with the proviso that R₇, R₈, R₉, R₁₀, and R₁₁ are not all H; and
with the proviso that the following compounds, or pharmaceutically acceptable salts thereof are excluded:

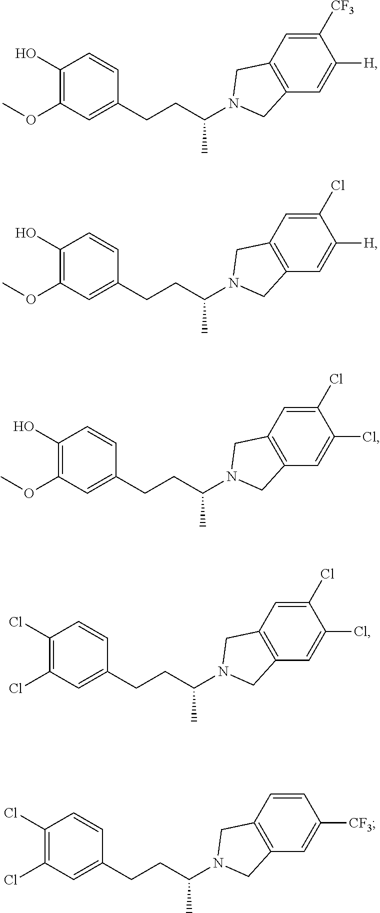

or
B. a compound of Formula IA

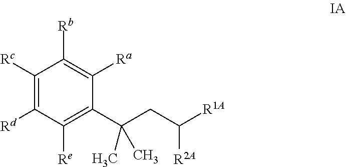

or pharmaceutically acceptable salt thereof:
wherein:
each of R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino;
R^{1A} is selected from the group consisting of hydrogen, alkyl, phenyl, or —CH═C(CH₃)₂; and
R^{2A} is an optionally substituted cyclic amino group.

### Compounds of Formula I

In some embodiments a compound for use in the invention is selected from a compound of Formula I

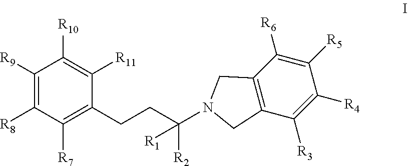

or a pharmaceutically-acceptable salt thereof:
wherein:
each of R₁ and R₂ is independently selected from H, C₁-C₆ alkyl, or CH₂OR′; wherein each R′ if present in R₁, and R₂ is independently H or C₁-C₆ alkyl; each of R₃, R₄, R₅, and R₆ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁-C₆ alkoxy C₁-C₆alkyl, aryl, heteroaryl, C₃-C₇ cycloalkyl, heterocycloalkyl, alkylaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₃, R₄, R₅, and R₆ is independently selected from the group consisting of H, CH₃, CH₂CH₃, C₃-C₆ alkyl, C₁-C₆ haloalkyl, or optionally substituted aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂, wherein the optionally substituted group is selected from C₁-C₆ alkyl or C₂-C₇ acyl;
or R₃ and R₄, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆haloalkyl, C₁-C₆ alkoxy, C₁-C₆haloalkoxy, aryl, arylalkyl, heteroaryl, heteroarylalkyl, cycloalkyl, and heterocycloalkyl or R₃ and R₄ are linked together to form a —O—C₁-C₂ methylene-O— group;
or R₄ and R₅, together with the C atom to which they are attached form a 4-, 5-6-7- or 8-membered cycloalkyl, aryl, heteroaryl, or heterocycloalkyl that is optionally substituted with 1, 2, 3, 4, or 5 substituents independently selected from OH, amino, halo, C₁. C₆ alkyl, C₁-C₆haloalkyl, C₁-C₆ alkoxy, C₁-C₆haloalkoxy, aryl, arylalkyl, heteroaryl, heteroarylalkyl, cycloalkyl, and heterocycloalkyl or R₄ and R₅ are linked together to form a —O—C₁₋₂ methylene-O— group;
each of R₇, R₈, R₉, R₁₀, and R₁₁ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁-C₆ alkoxy C₁-C₆ alkyl, aryl, heteroaryl, C₃-C₇ cycloalkyl, heterocycloalkyl, alkylaryl, heteroaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₇, R₈, R₉, R₁₀, and R₁₁ is independently selected from the group consisting of H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂;
or R₇ and R₈, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₇ and R₈ are linked together to form a —O—C₁₋₂ methylene-O— group;
or R₈ and R₉, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₈ and R₉ are linked together to form a —O—C₁₋₂ methylene-O— group;
each n is independently 0, 1, or 2;
with the proviso that R₇, R₈, R₉, R₁₀, and R₁₁ are not all H; and
with the proviso that the following compounds, or pharmaceutically acceptable salts thereof are excluded:

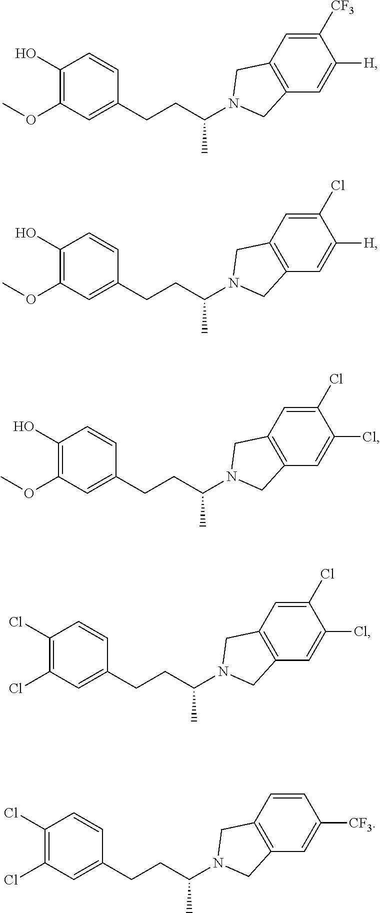

In some embodiments, a compound for use in the invention, is a compound of Formula I, or pharmaceutically acceptable salt thereof, wherein R₁ and R₂ are each independently selected from H or CH₃; R₃, R₄, R₅, and R₆ are each independently selected from H, C₁-C₆ alkyl, OH, OCH₃, O(C₁-C₆ alkyl), O(C₁-C₆ haloalkyl), F, Cl, CF₃, aryl, heteroaryl, C₃₋₇ cycloalkyl, CO₂R′, C(O)R′, OC(O)N(R′)₂, CONR′₂, NC(O)R′, NS(O)ₙR′, S(O)ₙNR′₂, S(O)ₙR′; where n=0, 1, or 2; R′ are each independently H, CH₃, CH₂CH₃, C₃-C₆ alkyl, C₁-C₆ haloalkyl; or optionally substituted piperazin-1-yl, piperidin-1-yl, morpholinyl, heterocycloalkyl, or aryl, wherein optionally substituted group is selected from C₁-C₆ alkyl or C₂-C₇ acyl; or R₃ and R₄, together with the C atom to which they are attached, form a 5-, or 6-membered C₃₋₇cycloalkyl, or aryl; or R₄ and R₅, together with the C atom to which they are attached, form a C₃₋₇cycloalkyl, or a 5- or 6-membered aryl; or R₃ and R₄ are linked together to form a —O—C₁₋₂ methylene-O— group; or R₄ and R₅ are linked together to form a —O—C₁₋₂ methylene-O— group; and R₇, R₈, R₉, R₁₀, and R₁₁ are each independently selected from H, OH, CH₃, CH₂CH₃, F, Cl, CF₃, OCF₃, C₁-C₆ haloalkyl, OCH₃, O(C₁-C₆ alkyl), OCH₂CH₂OH, O(C₁-C₆ alkyl)OH, aryl, heteroaryl, C₃₋₇ cycloalkyl, alkylaryl, CO₂R′, CONR′₂, S(O)ₙNR′₂, S(O)ₙR′, C(O)O(C₁₋₄ alkyl), OC(O)N(R′)₂, and C(O)NH(C₁₋₄ alkyl); where n=0, 1, or 2; R′ are each independently H, C₁-C₆ alkyl, C₁-C₆ haloalkyl, aryl, alkylaryl, or C₁₋₆ alkoxy.

In some embodiments, a compound for use in the invention, a compound for use in the invention, is a compound of Formula I, or pharmaceutically acceptable salt thereof, wherein R₇, R₁₀, R₁₁ are each H; R₃ and R₄ are each independently selected from H, F, Cl, S(O)ₙR′, C(O)R′, wherein n=2, and R′ is selected from CH₃, piperazin-1-yl, piperidin-1-yl, morpholinyl; R₈ is selected from OH, OCH₃, OCH(CH₃)₂, OCH₂CH(CH₃)₂, or OC(CH₃)₃; and R₉ is OH.

In some embodiments, a compound for use in the invention a compound for use in the invention, is a compound of Formula I, or pharmaceutically acceptable salt thereof, selected from the group consisting of:

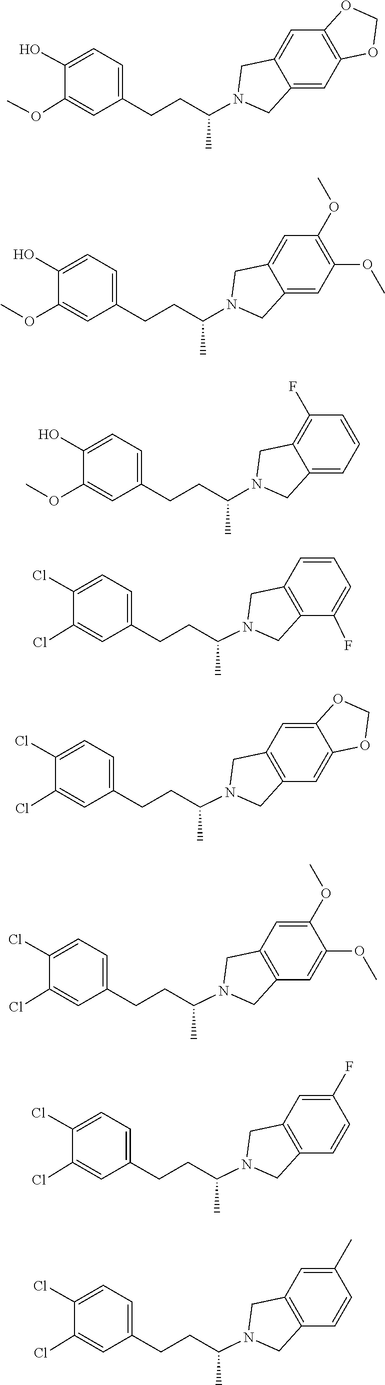

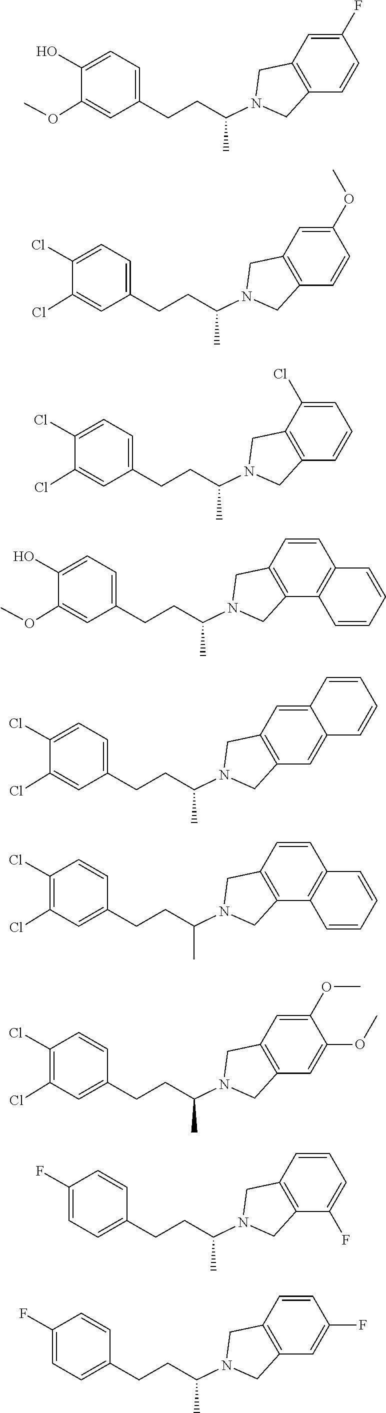

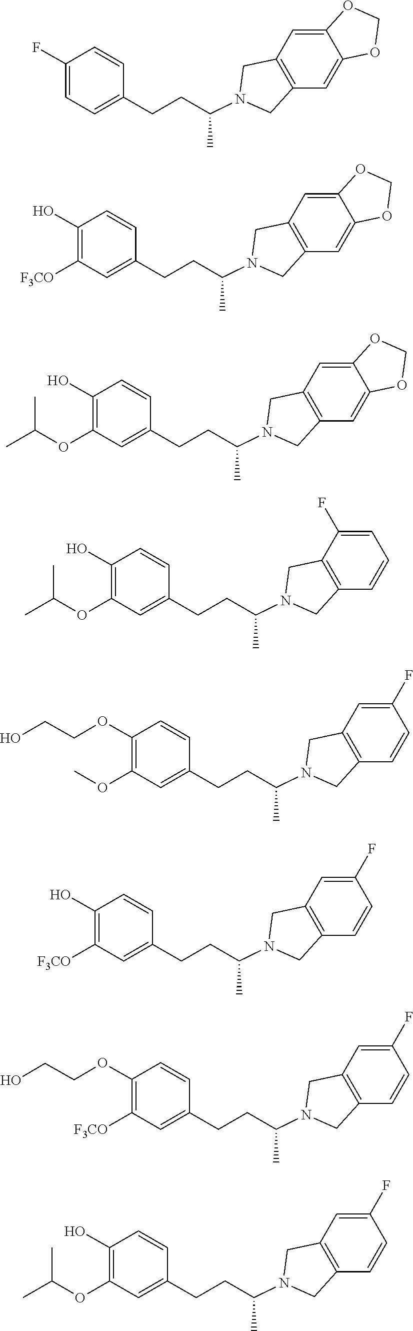

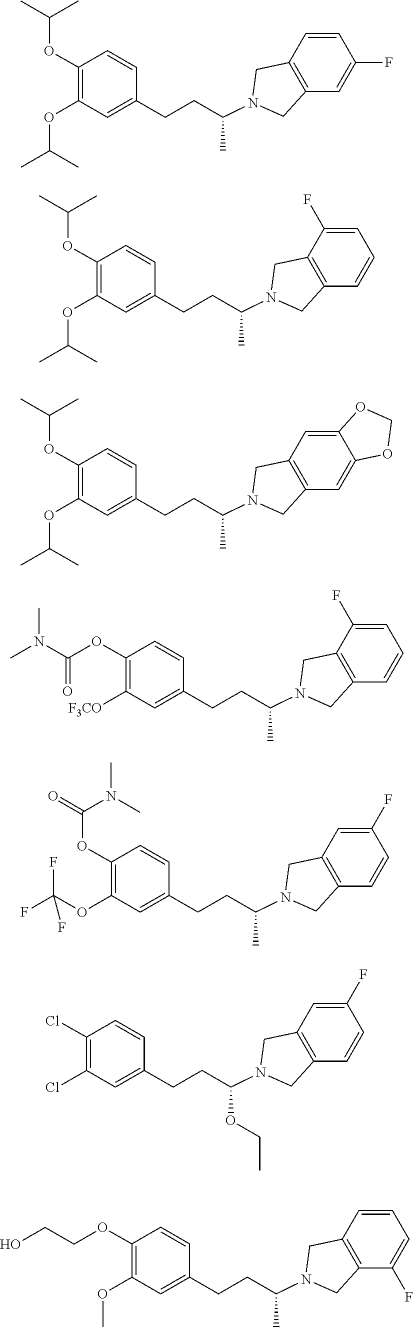

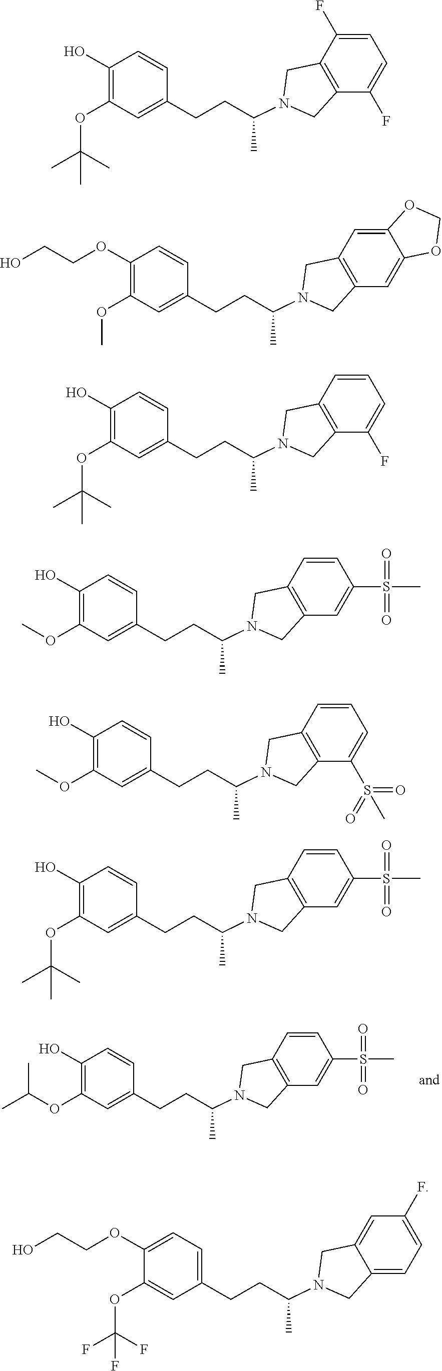

In further embodiments, a a compound for use in the invention a compound of Formula II, or pharmaceutically acceptable salt thereof:

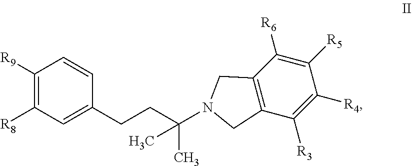

wherein R₃, R₄, R₅, and R₆ are each independently selected from H, Cl, F, OH, CH₃, C₁₋₆ alkyl, OCH₃, OCH(CH₃)₂, OCH₂CH(CH₃)₂, OC(CH₃)₃, OC₁₋₆ alkyl, aryl, heteroaryl, heterocycloalkyl, CO₂R′, CONR′₂, NC(O)R′, NS(O)ₙR′, S(O)ₙNR′₂, S(O)ₙR′, C(O)R′, OC(O)N(R′)₂, or C(O)NH(C₁₋₄ alkyl), wherein n=0, 1, or 2; and R′ are each independently H, C₁-C₆ alkyl, C₁-C₆ haloalkyl; or optionally substituted aryl, alkylaryl, piperazin-1-yl, piperidin-1-yl, morpholinyl, heterocycloalkyl, heteroaryl, C₁₋₆ alkoxy, NH(C₁₋₄ alkyl), or NH(C₁₋₄ alkyl)₂, wherein optionally substituted group is selected from C₁-C₆ alkyl or C₂-C₇ acyl;
or R₃ and R₄, together with the C atom to which they are attached, form a 6-membered aryl; or R₃ and R₄ are linked together to form a —O—C₁₋₂ methylene-O— group; or R₄ and R₅, together with the C atom to which they are attached, form a 6-membered aryl; or R₄ and R₅ are linked together to form a —O—C₁-2 methylene-O— group; and
R₈ and R₉ are each independently selected from H, Cl, F, OH, CH₃, C₁₋₆ alkyl, OCH₃, OCH(CH₃)₂, OCH₂CH(CH₃)₂, OC(CH₃)₃, O(CO)R′, OC₁₋₆ alkyl, aryl, heteroaryl, heterocycloalkyl, CO₂R′, CONR′₂, NC(O)R′, NS(O)ₙR′, S(O)ₙNR′₂, S(O)ₙR′, OC(O)N(R′)₂, or C(O)NH(C₁₋₄ alkyl);
or R₈ and R₉, together with the N or C atoms to which they are attached form a form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁₋₆alkyl, C₁₋₆ haloalkyl, C₁₋₆alkoxy, C₁₋₆ haloalkoxy, aryl, arylalkyl, heteroaryl, heteroarylalkyl, cycloalkyl, and heterocycloalkyl and R⁹ and R¹⁰ are each independently selected from a bond, C, N, S, and O; or R₈ and R₉ are linked together to form a —O—C₁₋₂ methylene-O— group.

In further embodiments, a compound for use in the invention, is a compound of Formula II, or pharmaceutically acceptable salt thereof, wherein at least one of R₃, R₄, R₅ and R₆ is not H; and at least one of R₈ and R₉ is not H.

In other embodiments, a compound for use in the invention, is a compound of Formula II, or pharmaceutically acceptable salt thereof, wherein R₇, R₁₀, R₁₁ are each H; R₃ and R₄ are each independently selected from H, F, Cl, S(O)ₙR′, C(O)R′, wherein n=2, and R′ is selected from CH₃, or optionally substituted piperazin-1-yl, piperidin-1-yl, or morpholinyl, wherein optionally substituted group is selected from C₁-C₆ alkyl or C₂-C₇ acyl; R₈ is selected from OH, C₁, OCH₃, OCH(CH₃)₂, OCH₂CH(CH₃)₂, or OC(CH₃)₃; and R₉ is OH or C₁.

In further embodiments, a compound for use in the invention, is a compound of Formula II, or pharmaceutically acceptable salt thereof, wherein R₃ and R₄ are each independently selected from H, F, Cl, S(O)ₙR′, C(O)R′, wherein n=2, and R′ is selected from CH₃, piperazin-1-yl, piperidin-1-yl, or morpholinyl; R₅ and R₆ are each H; R₈ is selected from OH, OCH₃, OCH(CH₃)₂, OCH₂CH(CH₃)₂, or OC(CH₃)₃; and R₉ is OH.

In further embodiments, a compound for use in the invention, or pharmaceutically acceptable salt thereof is selected from the group consisting of

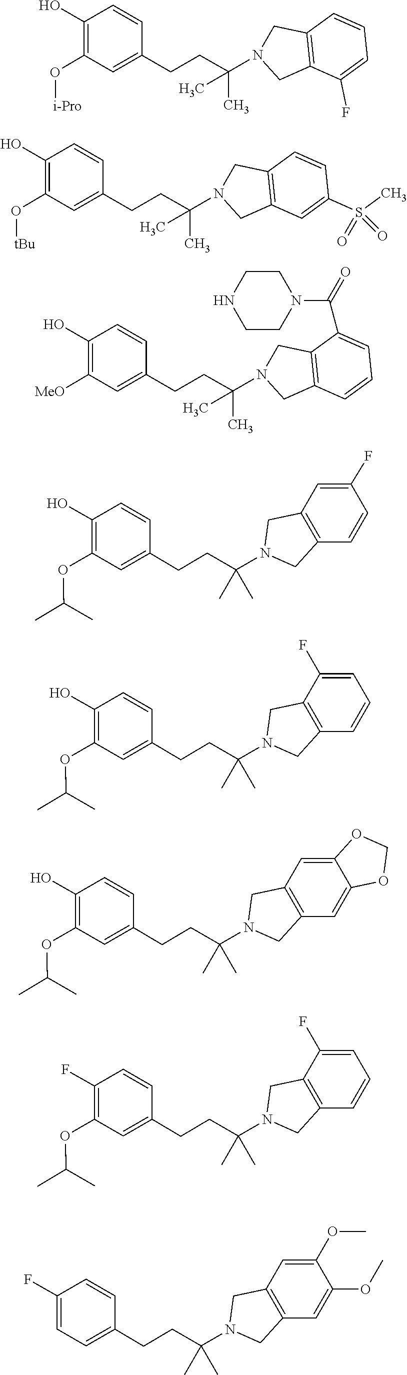

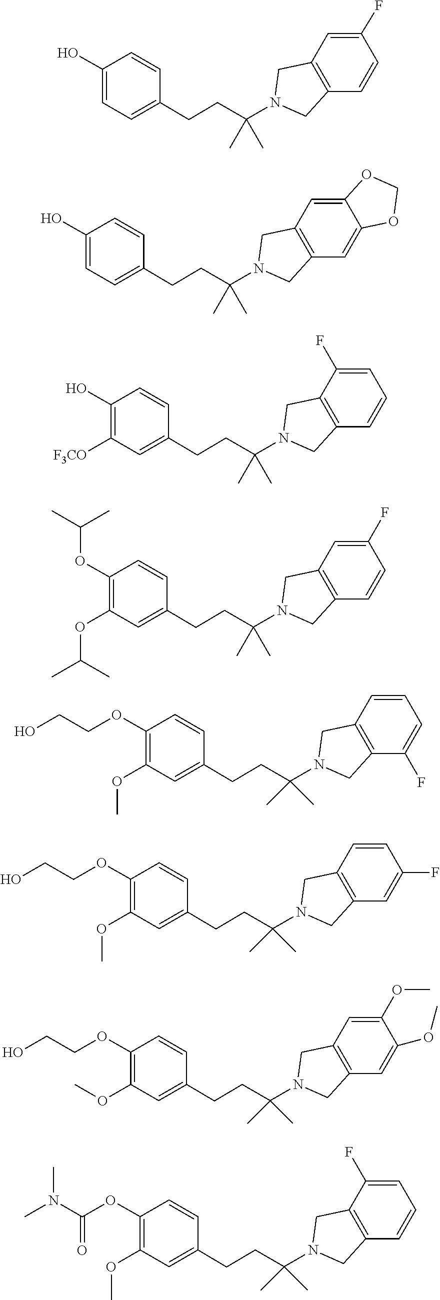

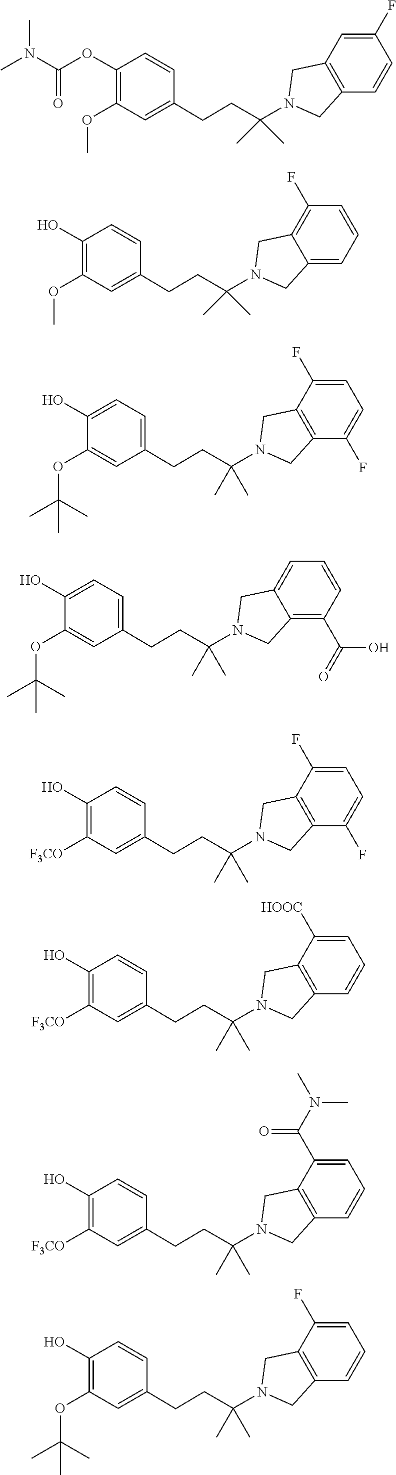

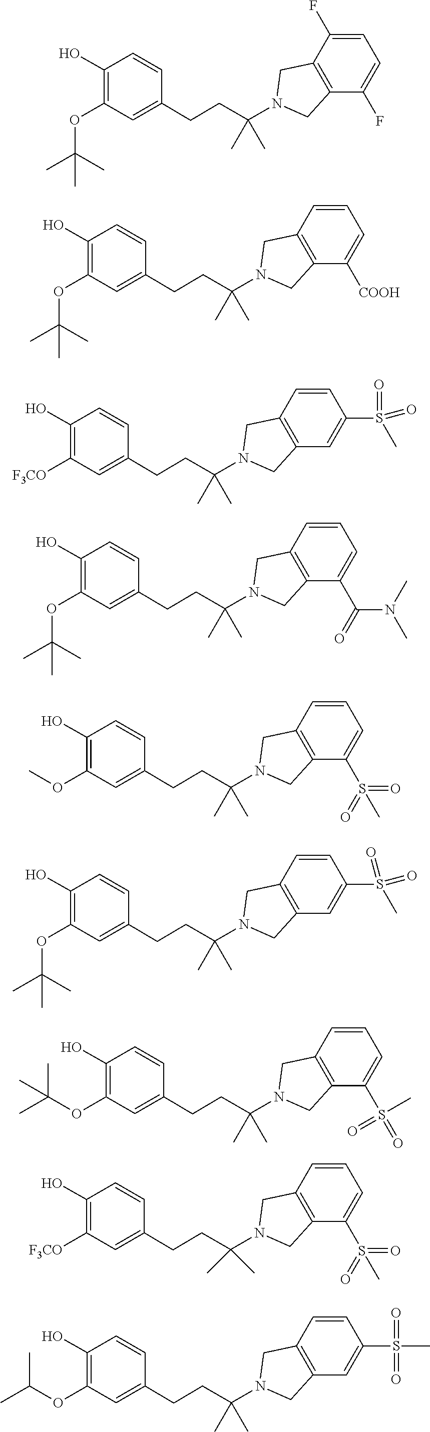

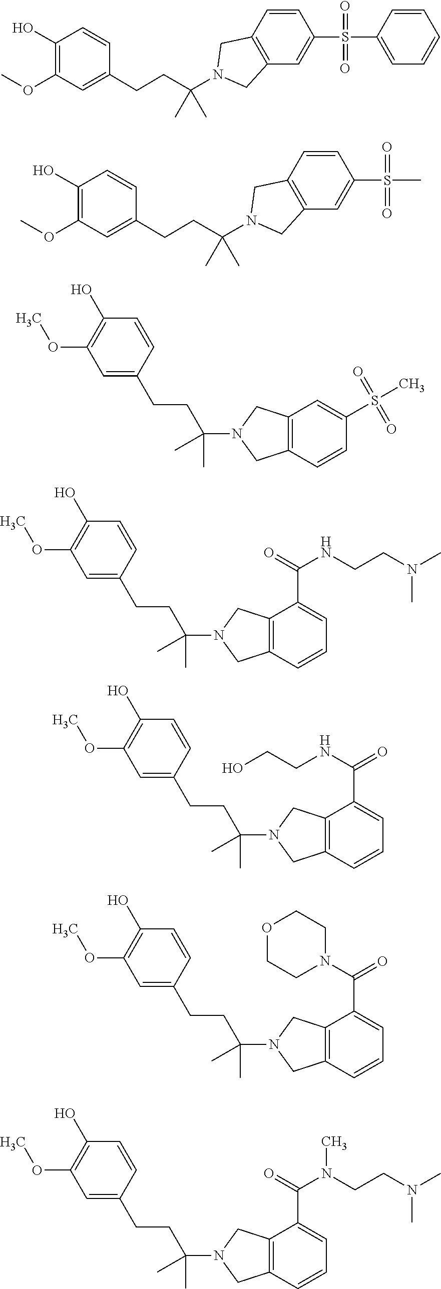

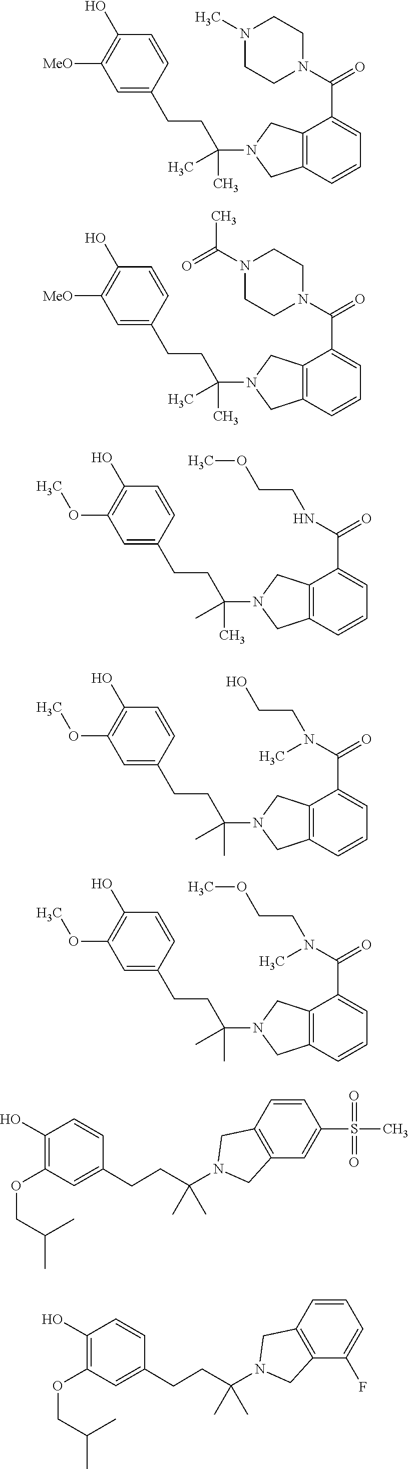

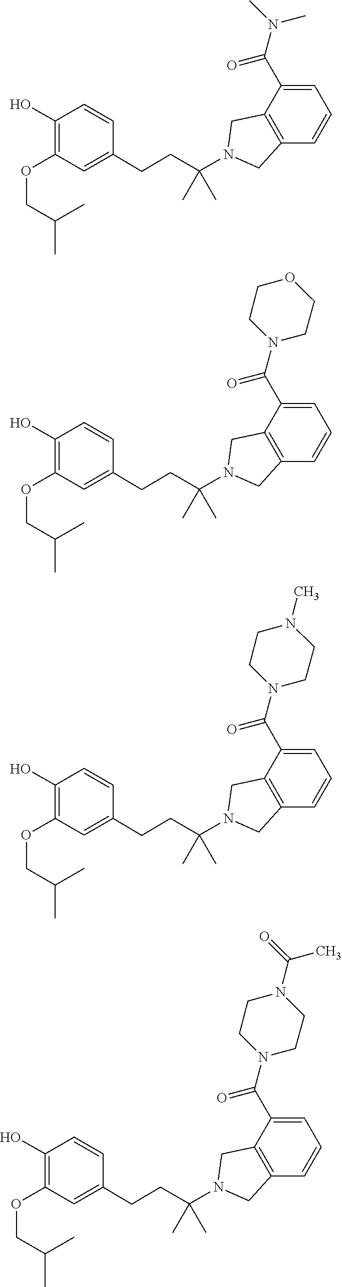 [SMILES: CC(C)COc1cc(CCC(C)(C)N2Cc3cccc(C(=O)N(C)C)c3C2)ccc1O ; CC(C)COc1cc(CCC(C)(C)N2Cc3cccc(C(=O)N4CCOCC4)c3C2)ccc1O ; CC(C)COc1cc(CCC(C)(C)N2Cc3cccc(C(=O)N4CCN(C)CC4)c3C2)ccc1O ; CC(=O)N1CCN(C(=O)c2cccc3c2CN(C(C)(C)CCc2ccc(O)c(OCC(C)C)c2)C3)CC1]

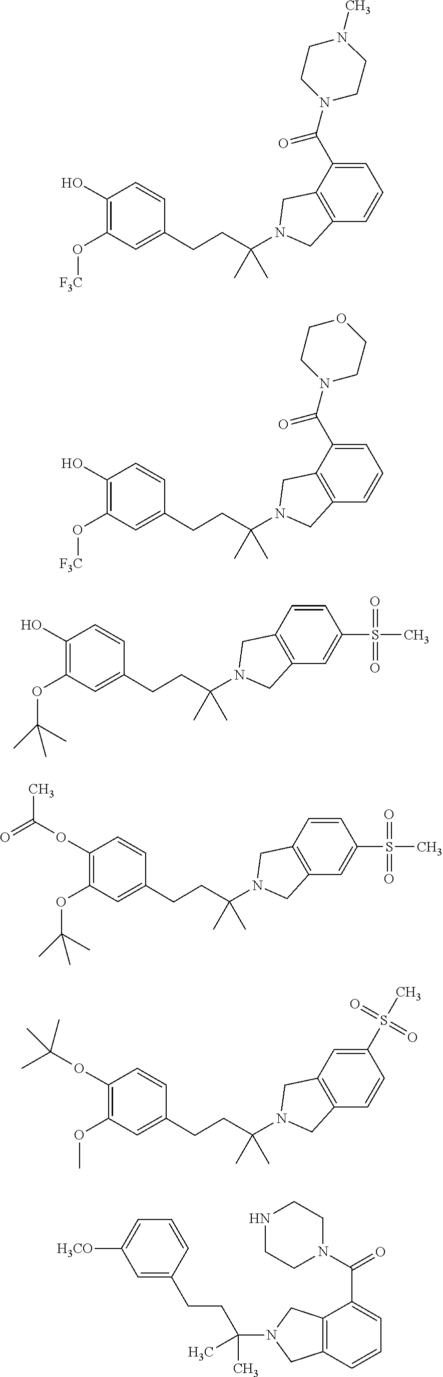

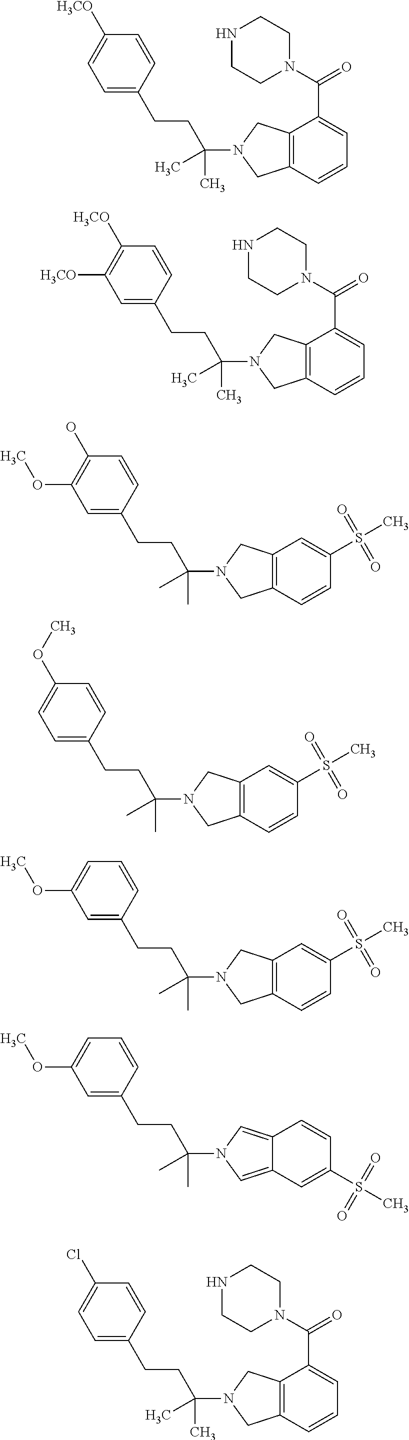

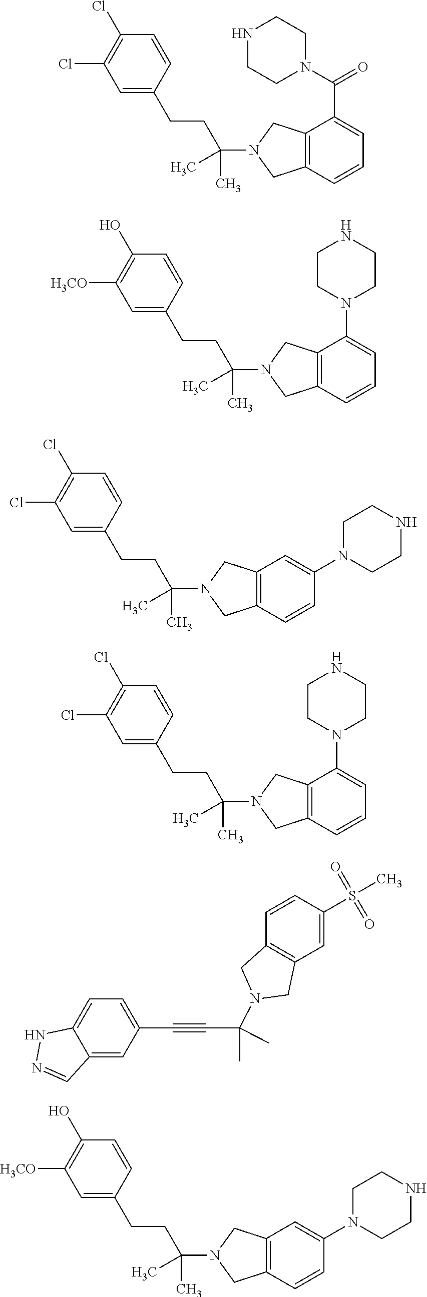

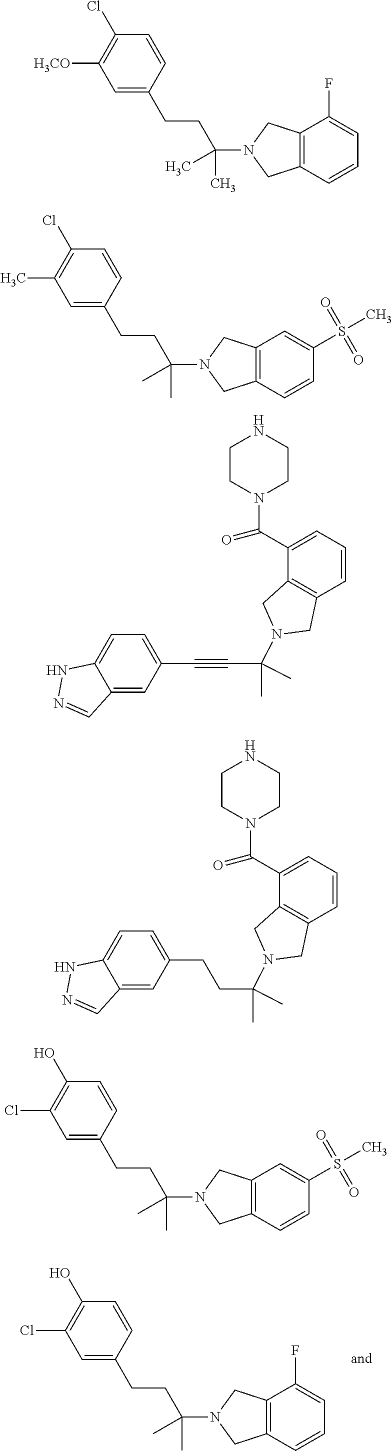

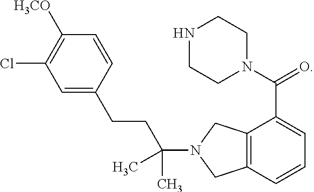 [SMILES: COc1ccc(CCC(C)(C)N2Cc3cccc(C(=O)N4CCNCC4)c3C2)cc1Cl]

In further embodiments, a compound for use in the invention, or pharmaceutically acceptable salt thereof, is a compound selected from the group consisting of:

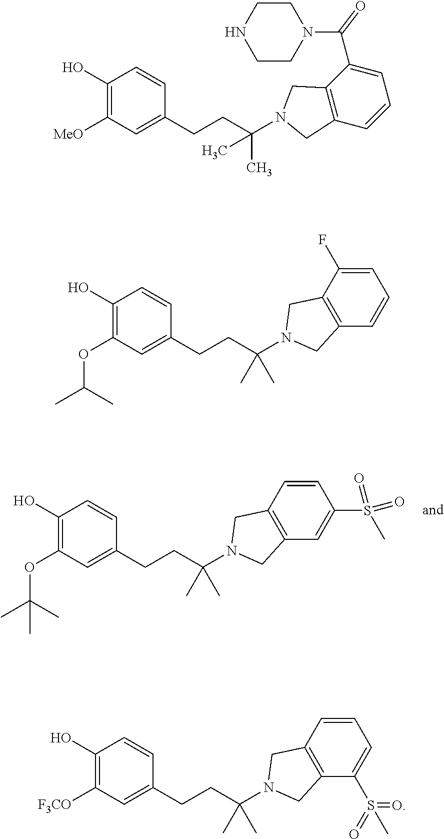 [SMILES: COc1cc(CCC(C)(C)N2Cc3cccc(C(=O)N4CCNCC4)c3C2)ccc1O ; CC(C)Oc1cc(CCC(C)(C)N2Cc3cccc(F)c3C2)ccc1O ; CC(C)(C)Oc1cc(CCC(C)(C)N2Cc3ccc(S(C)(=O)=O)cc3C2)ccc1O ; CC(C)(CCc1ccc(O)c(OC(F)(F)F)c1)N1Cc2cccc(S(C)(=O)=O)c2C1]

In further embodiments, a compound for use in the invention, or pharmaceutically acceptable salt thereof, is a compound selected from the group consisting of:

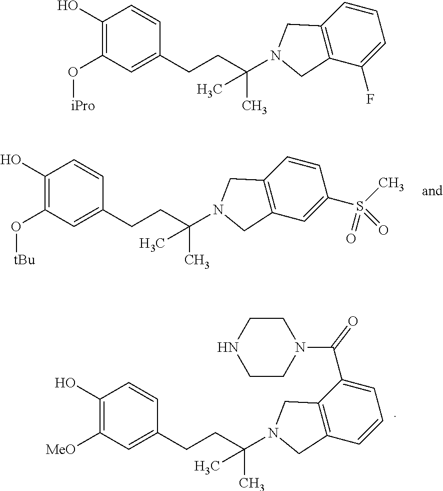

In some embodiments, a compound for use in the invention, is:

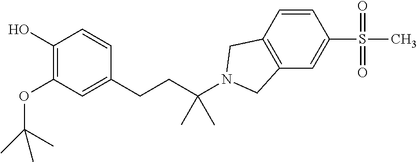 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments, a compound for use in the invention, is:

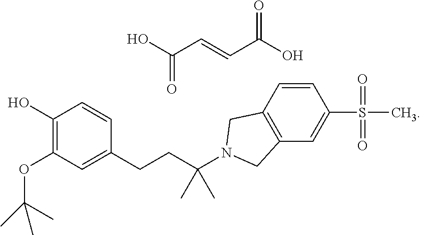

In some embodiments a compound for use in the invention, or pharmaceutically acceptable salt thereof, is a compound wherein each of R₁, R₂, R₃, R₄, R₅, R₆, R₇, R₈, R₉, R₁₀, and R₁₁ of Formula I is as defined herein, with the proviso that when R₁, R₃, R₆, R₇, R₁₀ and R₁₁ are each H; R₂ is CH₃; R₈ is OCH₃ or Cl; and R₉ is OH or Cl; then R₄ is not Cl or CF₃, and R₅ is not Cl or CF₃.

In some embodiments, a compound for use in the invention, is a compound of Formula II:

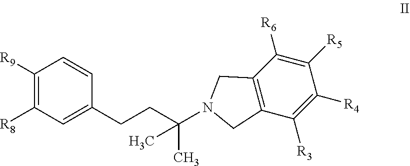

or a pharmaceutically acceptable salt thereof
wherein R₃, R₄, R₅, R₆, R₈, and R₉ are as described herein.

In another embodiment, a compound for use in the invention, is a compound of Formula III:

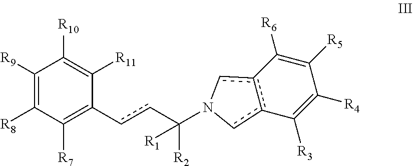

or pharmaceutically acceptable salt thereof, wherein R₃, R₄, R₅, R₆, R₇, R₈, R₉, R₁₀ and R₁₁ are as provided herein and wherein each [symbol] is independently selected from a single, double or triple bond.

In some aspects, a compound for use in the invention, is a compound according to Formula III selected from the group consisting of:

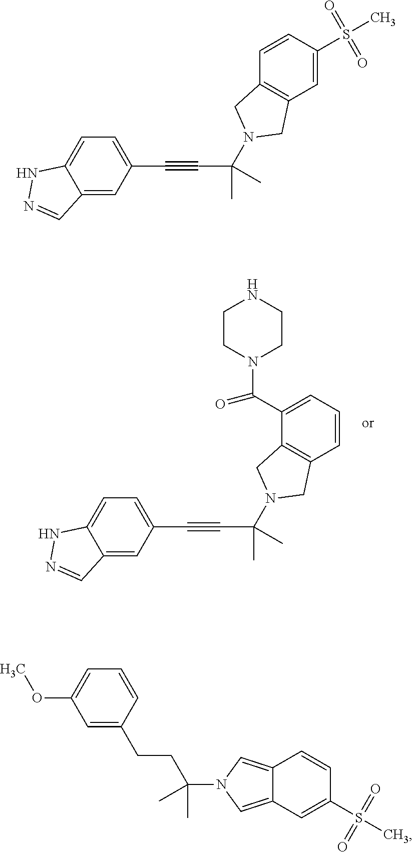

or a pharmaceutically acceptable salt thereof.

In some embodiments, a compound for use in the invention comprises a racemic mixture or an enantiomer of a compound of Formula I, wherein R₃, R₄, R₅, R₆, R₈, and R₉ are as described herein.

In some embodiments, a compound for use in the invention comprises a compound of Formula I, or a pharmaceutically acceptable salt thereof, wherein R₈ and R₉ are independently selected from OH, C₁₋₆ alkoxy, and hydroxy C₁₋₆ alkoxy.

In some embodiments, a compound for use in the invention comprises a compound of Formula I, or a pharmaceutically acceptable salt thereof, wherein R₈ and R₉ are independently selected from OH and NH(C₁₋₄ alkyl).

In some embodiments, a compound for use in the invention comprises a compound of Formula I, or a pharmaceutically acceptable salt thereof, wherein R₈ and R₉ are independently selected from H, halo, C₁₋₆ haloalkyl, and C₁₋₆ haloalkoxy.

In some embodiments, a compound for use in the invention comprises a compound of Formula I, or a pharmaceutically acceptable salt thereof, wherein R₈ and R₉ are each independently selected from OH, halo, C₁₋₆ alkoxy and C₁₋₆ haloalkoxy and R₁ and R₂ are each independently C₁₋₆ alkyl.

In some embodiments, a compound for use in the invention comprises a compound of Formula I, or a pharmaceutically acceptable salt thereof, wherein R₁ and R₂ are each methyl.

In some embodiments, a compound for use in the invention comprises a compound of Formula I, or a pharmaceutically acceptable salt thereof, wherein one of R₁ and R₂ is methyl and the other is H.

In some embodiments, a compound for use in the invention comprises a compound of Formula I, or a pharmaceutically acceptable salt thereof, wherein R₈ and R₉ are each independently selected from OH and C₁₋₆ alkoxy and R₁ and R₂ are each independently methyl.

In some embodiments, a compound for use in the invention comprises a compound of Formula I, or a pharmaceutically acceptable salt thereof, wherein R₈ and R₉ are independently selected from H, halo, and C₁₋₆ haloalkyl, and R₁ and R₂ are each methyl.

In some embodiments, a compound for use in the invention comprises a compound of Formula I, or a pharmaceutically acceptable salt thereof, wherein R₈ and R⁹ are each independently selected from H, halo and C₁₋₆ haloalkyl.

In some embodiments, a compound for use in the invention comprises a compound of Formula I, or a pharmaceutically acceptable salt thereof, wherein R₇ and R₁₁ are each H.

In some embodiments, a compound for use in the invention comprises a compound of Formula I, or a pharmaceutically acceptable salt thereof, wherein R₃, R₄, R₅, and R₆ are each independently selected from H, halo, C₁₋₆ alkyl, C₁₋₆ haloalkyl and C₁₋₆ alkoxy.

In some embodiments, a compound for use in the invention comprises a compound of Formula I, or a pharmaceutically acceptable salt thereof, wherein R₃, R₄ and R₅ are each independently selected from H, halo, C₁₋₆ alkyl, C₁₋₆ haloalkyl and C₁₋₆ alkoxy.

In some embodiments, a compound for use in the invention comprises a compound of Formula I, or a pharmaceutically acceptable salt thereof, wherein R₃, R₄, R₅, and R₆ are each independently selected from H, halo, S(O)ₙR′, C(O)OR′, C(O)N(R′)₂, and C(O)R′; where n=2; R′ are each independently H, CH₃, CH₂CH₃, C₃-C₆ alkyl, C₁-C₆ haloalkyl, or optionally C₁-C₆ alkyl or C₂-C₇ acyl substituted aryl, alkylaryl, piperazinyl, piperidinyl, morpholinyl, heterocycloalkyl, and heteroaryl.

In some embodiments, a compound for use in the invention comprises a compound of Formula I, or a pharmaceutically acceptable salt thereof, wherein R₃, R₄ and R₅ are each independently selected from H, halo, S(O)ₙR′, and C(O)R′; where n=2; R′ are each independently CH₃, CH₂CH₃, C₃-C₆ alkyl, aryl, piperazin-1-yl, piperidin-1-yl, and morpholinyl-4-yl.

In some embodiments, a compound for use in the invention comprises a compound of Formula I, or a pharmaceutically acceptable salt thereof, wherein R₃, R₄ and R₅ are each independently selected from H, halo, S(O)ₙR′, and C(O)R′; where n=2; R′ are each independently CH₃, CH₂CH₃, C₃-C₆ alkyl, aryl, piperazin-1-yl, piperidin-1-yl, and morpholinyl-4-yl; R₈ and R₉ are each independently selected from OH, halo, C₁₋₆ alkoxy and C₁₋₆ haloalkoxy; and R₁ and R₂ are each methyl.

In some embodiments, a compound for use in the invention comprises a compound of Formula I, or a pharmaceutically acceptable salt thereof, wherein R₃ and R₄ or R₄ and R₅ together with the C atom to which they are attached form a 6-membered cycloalkyl, or a heterocycloalkyl, aryl or heteroaryl ring.

In some embodiments, a compound for use in the invention comprises a compound of Formula I, or a pharmaceutically acceptable salt thereof, wherein R₃ and R₄ or R₄ and R₅ are O, and are linked together to form a —O—C₁₋₂ methylene-O— group.

In some embodiments, a compound for use in the invention comprises a compound of Formula I, or a pharmaceutically acceptable salt thereof, wherein R² and R³ are independently selected from H, OH, halo, C₁₋₆ alkoxy and C₁₋₆ haloalkyl.

In some embodiments, a compound for use in the invention comprises a compound of Formula II, or a pharmaceutically acceptable salt thereof, wherein R₃ and R₄ are independently selected from H, Cl, F, —OMe, —CF₃, S(O)ₙR′, and C(O)R′; where n=2; R′ are each independently H, CH₃, CH₂CH₃, C₃-C₆ alkyl, aryl, piperazin-1-yl, piperidin-1-yl, and morpholinyl-4-yl; R₈ and R₉ are each independently selected from OH and C₁₋₆ alkoxy.

In some embodiments, a compound for use in the invention comprises a compound of Formula I, or a pharmaceutically acceptable salt thereof, wherein R² and R³ are independently selected from H, OH, Cl, F, —OMe, and —CF₃, wherein R² and R³ are each independently selected from H and C₁₋₆ alkyl, wherein R⁹ is H, and wherein R⁵ and R⁶ are each independently selected from H and C₁₋₆ haloalkyl.

In some embodiments, a compound of any of Formulas I-III according to any embodiment described herein, may contain a proviso to remove one or more of the following compounds:

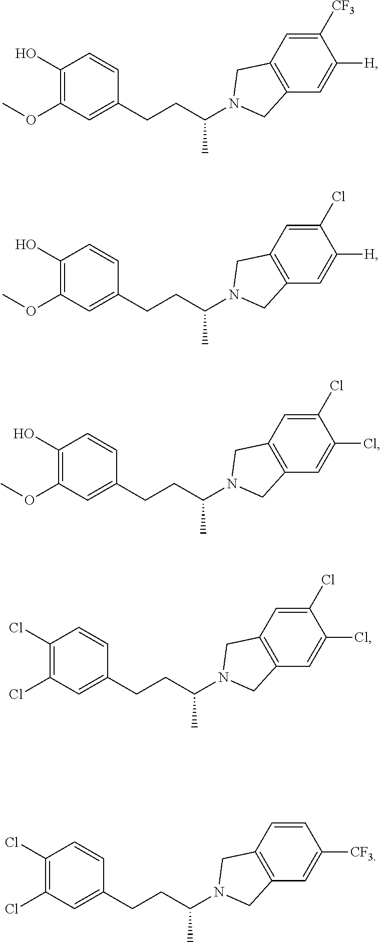

### Compounds of Formula IA

In some embodiments a compound for use in the invention comprises a compound of Formula IA

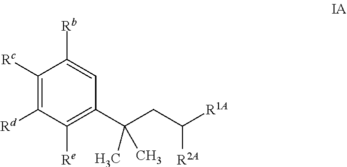

or pharmaceutically acceptable salt thereof:
wherein:
each of R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino;
R^{1A} is selected from the group consisting of hydrogen, alkyl, phenyl, or —CH═C(CH₃)₂; and
R^{2A} is an optionally substituted cyclic amino group.

In some embodiments, a compound for use in the invention comprises a compound of Formula IA wherein each of substituents R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino.

In some embodiments, a compound for use in the invention comprises a compound of Formula IA wherein each of substituents R^{a}, R^{b}, R^{c}, R^{d} and R^{e} is independently selected from the group consisting of, H, Cl, F, and CF₃.

In some embodiments, a compound for use in the invention comprises a compound of Formula IA wherein each of substituents R^{a}, R^{b}, R^{d} and R^{e} is independently H; and R^{c} is selected from the group consisting of H, hydroxyl, halo, alkyl, alkoxy, CF₃, SO₂CH₃, and morpholino.

In some embodiments, a compound for use in the invention comprises a compound of Formula IA wherein each of substituents R^{a}, R^{b}, R^{d} and R^{e} is independently H; and R^{c} is selected from the group consisting of H, hydroxyl, Cl, F, methyl, —OCH₃, —OC(CH₃)₃, O—CH(CH₃)₂, CF₃, SO₂CH₃, and morpholino.

In some embodiments, a compound for use in the invention comprises a compound of Formula IA wherein each of substituents R^{a}, R^{b}, R^{d} and R^{e} is independently H; and R^{c} is selected from the group consisting of H, Cl, F, and CF₃.

In various embodiments, a compound for use in the invention comprises a compound of Formula IA wherein R^{2A} is any heterocycloalkyl or heteroaryl containing a nitrogen in the ring that is bound to the aliphatic chain of Formula IA through the nitrogen atom. In some embodiments, for example, R^{2A} is an optionally substituted cyclic amino group selected from:

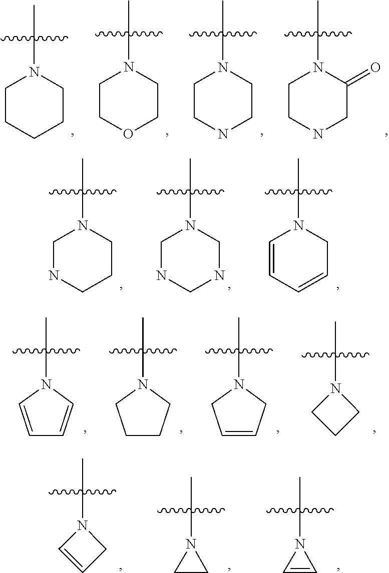

and the like, wherein each nitrogen containing heterocycloalkyl or heteroaryl can be optionally substituted with one or more substituents selected from, hydroxyl, halo, CF₃, alkoxy, aryloxy, optionally substituted C₁-C₁₀ alkyl, optionally substituted C₅-C₁₀ aryl, optionally substituted C₃-C₁₀ heteroaryl, substituted or unsubstituted C₃-C₁₀ cycloalkyl or heterocycloalkyl.

In various embodiments, a compound for use in the invention comprises a compound of Formula IA wherein R^{2A} is selected from the group consisting of optionally substituted aziridinyl, optionally substituted pyrrolidinyl, optionally substituted imidizolidinyl, optionally substituted piperidinyl, optionally substituted piperazinyl, optionally substituted oxopiperazinyl, and optionally substituted morpholinyl.

In some embodiments, a compound for use in the invention comprises a compound of Formula IA wherein when R^{2A} is a substituted cyclic amino, one or more of the hydrogen atoms in the cyclic amino group is replaced with a group selected from alkanoyl, alkoxy, alkoxyalkyl, (alkoxy)alkoxyalkyl, alkoxycarbonyl, alkyl, aryloxy, aryloyl, cycloalkanoyl, —OC(O)NCH(CH₃)₂, (N,N-dimethylamino)pyridinyl, (N,N-dimethylamino)sulfonyl, halo, heterocyclyl, (heterocyclyl)alkoxyalkyl, hydroxyl, hydroxyalkyl, methylpiperidinyl, methylsulfonyl, methylsulfonylphenyl, morpholinylpyridinyl, perfluoroalkyl, phenyl, piperidinyl, pyrrolidinylpyridinyl, tetrahydropyranyl, and CF₃. In some embodiments two hydrogen atoms on the same carbon of the cyclic amino group are replaced with a compound selected from

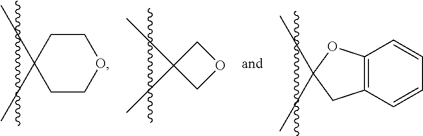

and to form a spiro compound.

In some embodiments, a compound for use in the invention comprises a compound of Formula IA wherein R^{2A} is a pyrrolidinyl or a substituted pyrrolidinyl substituted with one or more substituents selected from the group consisting of alkoxyalkyl, alkoxycarbonyl, alkyl, hydroxyl, and hydroxyalkyl. In some embodiments R^{2A} is a substituted pyrrolidinyl substituted with a single substituent selected from the group consisting of alkoxyalkyl, alkoxycarbonyl, alkyl, hydroxyl, and hydroxyalkyl. In some embodiments R² is a substituted pyrrolidinyl substituted with a single substituent selected from the group consisting of hydroxyl, hydroxymethyl, methoxymethyl, methoxycarbonyl and methyl.

In some embodiments, a compound for use in the invention comprises a compound of Formula IA wherein R^{2A} is a piperidinyl or a substituted piperidinyl substituted with one or more substituents selected from the group consisting of alkoxy, alkoxyalkyl, (alkoxy)alkoxyalkyl, alkoxycarbonyl, alkyl, aryloxy, —OC(O)NCH(CH₃)₂, (N,N-dimethylamino)pyridinyl, halo, heterocyclyl, (heterocyclyl)alkoxyalkyl, hydroxy, hydroxyalkyl, methylpiperidinyl, methylsulfonylphenyl, morpholinylpyridinyl, perfluoroalkyl, phenyl, piperidinyl, pyrrolidinylpyridinyl, tetrahydropyranyl, and CF₃. In some embodiments, R^{2A} is a piperidinyl or a substituted piperidinyl substituted with a single substituent selected from the group consisting of alkoxy, alkoxyalkyl, (alkoxy)alkoxyalkyl, alkoxycarbonyl, alkyl, aryloxy, —OC(O)NCH(CH₃)₂, (N,N-dimethylamino)pyridinyl, halo, heterocyclyl, (heterocyclyl)alkoxyalkyl, hydroxyl, hydroxyalkyl, methylpiperidinyl, methylsulfonylphenyl, morpholinylpyridinyl, perfluoroalkyl, phenyl, piperidinyl, pyrrolidinylpyridinyl, tetrahydropyranyl, and CF₃. In some embodiments, R^{2A} is a piperidinyl or a substituted piperidinyl substituted with a single substituent selected from the group consisting of alkoxy, alkoxyalkyl, (alkoxy)alkoxyalkyl, alkoxycarbonyl, alkyl, aryloxy, —OC(O)NCH(CH₃)₂, (N,N-dimethylamino)pyridinyl, halo, heterocyclyl, (heterocyclyl)alkoxyalkyl, hydroxyl, hydroxyalkyl, methylpiperidinyl, methylsulfonylphenyl, morpholinylpyridinyl, perfluoroalkyl, phenyl, piperidinyl, pyrrolidinylpyridinyl, tetrahydropyranyl, and CF₃. In some embodiments, R^{2A} is a piperidinyl or a substituted piperidinyl substituted with a single substituent selected from the group consisting of methyl, isopropyl, isobutyl, CF₃, hydroxymethyl, hydroxyethyl, (isopropyloxy)ethyl, —(CH₂)₂O(CH₂)₂OCH₃, —(CH₂)₃OCH₃, —C(O)OMe, —C(O)OEt, hydroxyl, methoxy, isopropyloxy, phenyloxy, F, ethoxy, phenyl,

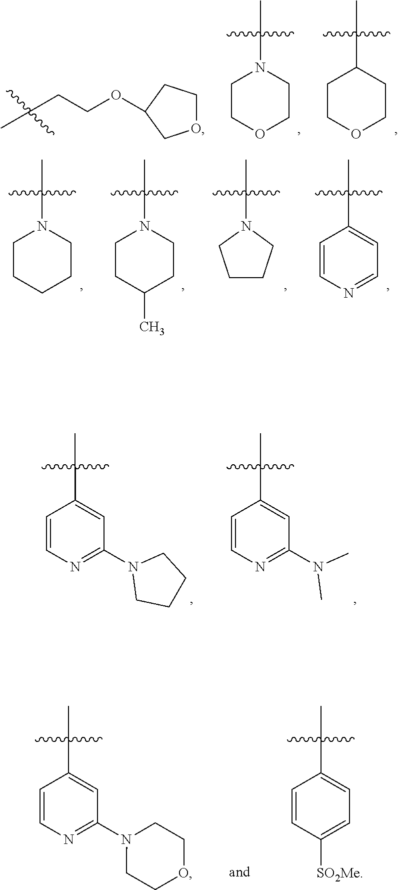

In some embodiments, R^{2A} is a piperidinyl or a substituted piperidinyl substituted at the 4 position of the piperidinyl with a single substituent selected from the group consisting of alkoxy, alkoxyalkyl, (alkoxy)alkoxyalkyl, alkoxycarbonyl, alkyl, aryloxy, —OC(O)NCH(CH₃)₂, (N,N-dimethylamino)pyridinyl, halo, heterocyclyl, (heterocyclyl)alkoxyalkyl, hydroxyl, hydroxyalkyl, methylpiperidinyl, methylsulfonylphenyl, morpholinylpyridinyl, perfluoroalkyl, phenyl, piperidinyl, pyrrolidinylpyridinyl, tetrahydropyranyl, and CF₃. In some embodiments, R^{2A} is a piperidinyl or a substituted piperidinyl substituted at the 4 position of the piperidinyl with a single substituent selected from the group consisting of methyl, isopropyl, isobutyl, CF₃, hydroxymethyl, hydroxyethyl, (isopropyloxy)ethyl, —(CH₂)₂O(CH₂)₂OCH₃, —(CH₂)₃OCH₃, —C(O)OMe, —C(O)OEt, hydroxyl, methoxy, isopropyloxy, phenyloxy, F, ethoxy, phenyl,

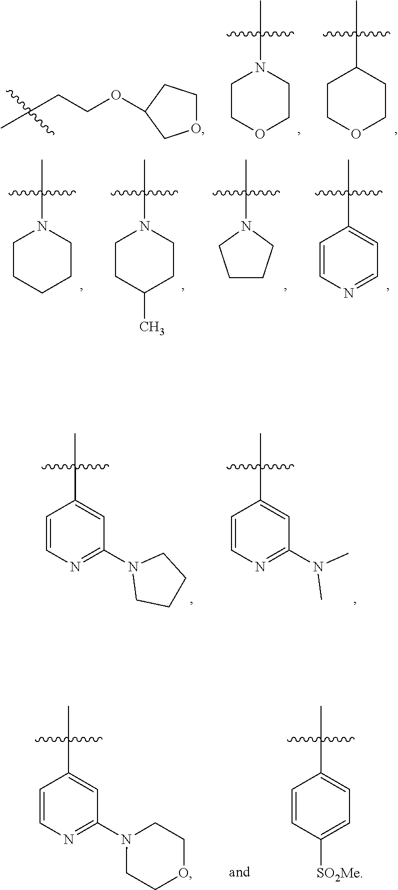

In some embodiments, a compound for use in the invention comprises a compound of Formula IA wherein R^{2A} is a piperidinyl or a substituted piperidinyl substituted with two substituent groups on the same carbon of the piperidinyl independently selected from the group consisting of alkoxyalkyl, alkyl, —OC(O)NCH(CH₃)₂, hydroxyl, and phenyl. In some embodiments, R^{2A} is a piperidinyl or a substituted piperidinyl substituted with two substituent groups at the 4 position of the piperidinyl independently selected from the group consisting of alkoxyalkyl, alkyl, —OC(O)NCH(CH₃)₂, hydroxyl, and phenyl. In some embodiments R² is a piperidinyl or a substituted piperidinyl substituted with two substituent groups at the 4 position selected from the group consisting of hydroxyl and methyl; hydroxyl and ethyl; hydroxyl and —(CH₂)₂OCH₃; hydroxyl and phenyl; methyl and phenyl; methyl and —OC(O)NCH(CH₃)₂; and butyl and —OC(O)NCH(CH₃)₂. In some embodiments two hydrogen atoms on the same carbon of the piperidinyl are replaced with a compound selected from

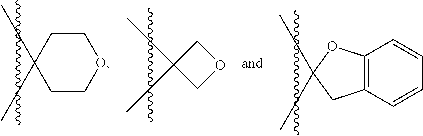

to form a spiro compound. In some embodiments two hydrogen atoms at the 4 position of the piperidinyl are replaced with a compound selected from

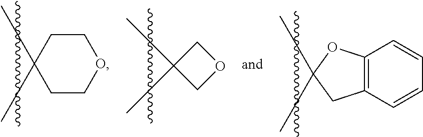

to form a spiro compound.

In some embodiments, a compound for use in the invention comprises a compound of Formula IA wherein R_{2A} is a piperazinyl or a substituted piperazinyl substituted with one or more substituents selected from the group consisting of alkanoyl, alkoxycarbonyl, aryloyl, cycloalkanoyl, (N,N-dimethylamino)sulfonyl, heterocyclyl, methylsulfonyl, and phenyl. In some embodiments, R^{2A} is a substituted piperazinyl substituted with a single substituent selected from the group consisting of alkanoyl, alkoxycarbonyl, aryloyl, cycloalkanoyl, (N,N-dimethylamino)sulfonyl, heterocyclyl, methylsulfonyl, and phenyl. In some embodiments, R^{2A} is a substituted piperazinyl substituted with a single substituent selected from the group consisting of —C(O)OC(CH₃)₃, —C(O)OCH₂CH(CH₃)₂, —C(O)OCH₂CH₃, —C(O)OCH₃, phenyl, —C(O)CH₃, —C(O)Ph, —SO₂Me, —SO₂N(CH₃)₂,

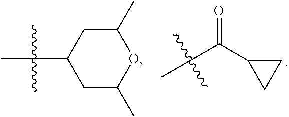

In some embodiments, R^{2A} is a substituted piperazinyl substituted with a single substituent at the 4 position selected from the group consisting of —C(O)OC(CH₃)₃, —C(O)OCH₂CH(CH₃)₂, —C(O)OCH₂CH₃, —C(O)OCH₃, phenyl, —C(O)CH₃, —C(O)Ph, —SO₂Me, —SO₂N(CH₃)₂,

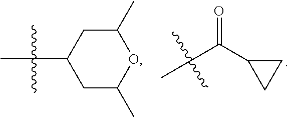

In certain embodiments, a compound for use in the invention comprises a compound of Formula IA wherein R^{2A} is a substituted piperdinyl of formula:

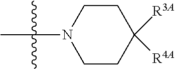

wherein, R^{3A} is hydrogen or C₁-C₈ alkyl, and R^{4A} is hydrogen, hydroxyl, halogen, CF₃, alkoxy, aryloxy, optionally substituted C₁-C₁₀ alkyl, optionally substituted C₅-C₁₀ aryl, optionally substituted C₃-C₁₀ heteroaryl, optionally substituted C₃-C₁₀ cycloalkyl or optionally substituted C₃-C₁₀ heterocycloalkyl.

In some embodiments, a compound for use in the invention comprises a compound of Formula IA wherein R^{2A} is

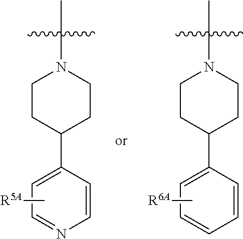

wherein each of R^{5A} and R^{6A} is independently, hydrogen, hydroxyl, sulfonyl, dialkylamino, optionally substituted C₁-C₁₀ alkyl, optionally substituted C₅-C₁₀ aryl optionally substituted C₃-C₁₀ heteroaryl, optionally substituted C₃-C₁₀ cycloalkyl or optionally substituted C₃-C₁₀ heterocycloalkyl. In some embodiments R^{5A} is hydrogen, dialkylamino, or C₃-C₁₀ heterocycloalkyl. In some embodiments R^{5A} is hydrogen, dialkylamino, pyrrolidinyl or morpholinyl. In some embodiments, R^{6A} is sulfonyl. In some embodiments, R^{6A} is methylsulfonyl.

In some embodiments, a compound for use in the invention comprises a compound of Formula IA wherein R^{2A} is:

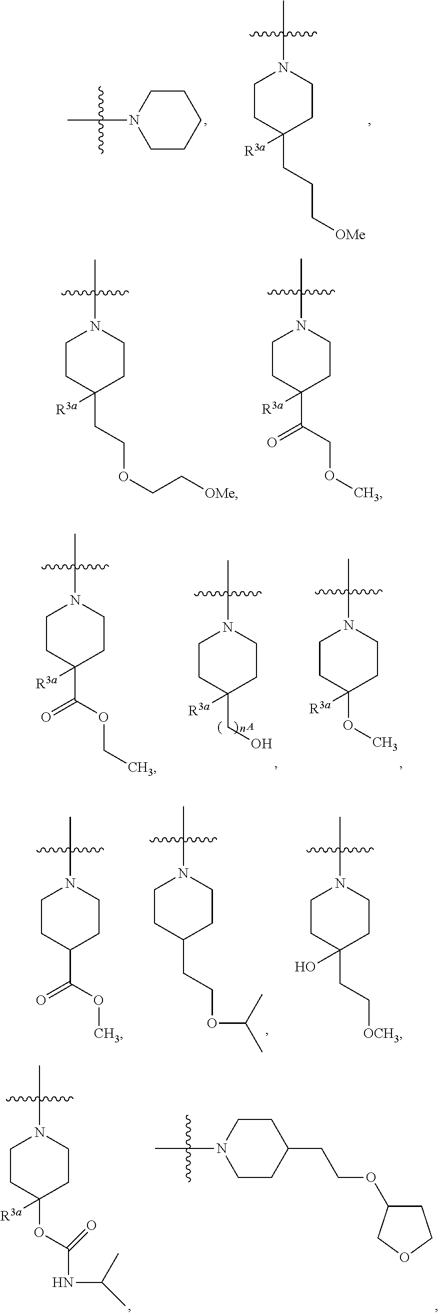

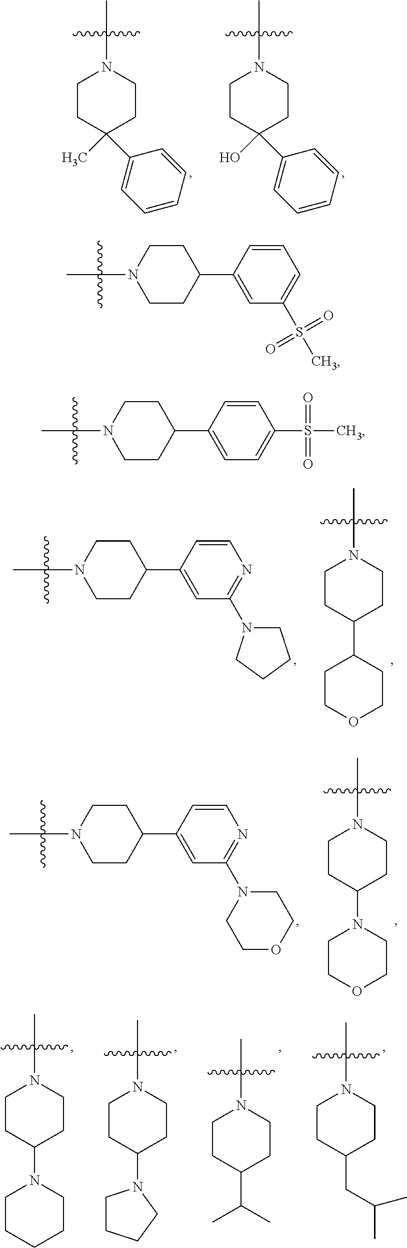

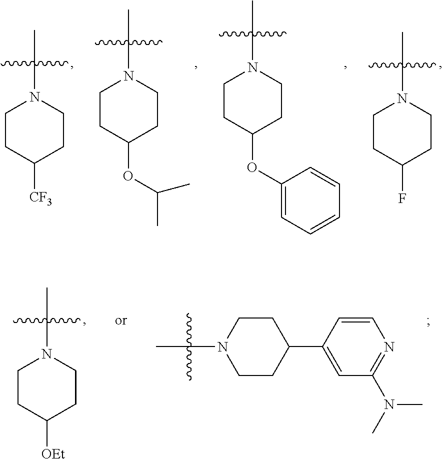

wherein R^{3a} selected from the group consisting of hydrogen and C₁-C₈ alkyl; and n^{A} is an integer selected from 0, 1 and 2.

In some embodiments a compound for use in the invention comprises a compound of Formula IA wherein R^{2A} is

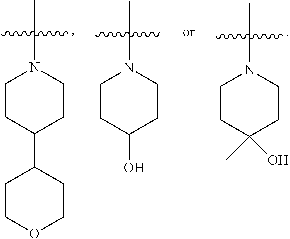

In some embodiments, a compound for use in the invention comprises a compound of Formula IA wherein R^{2A} is optionally substituted morpholinyl. In some embodiments, R^{2A} is morpholinyl.

In some embodiments a compound for use in the invention comprises a compound of Formula IA wherein R^{2A} or is optionally substituted piperazinyl of the formula

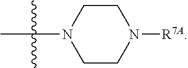

wherein R⁷ is hydrogen, hydroxyl, sulfonyl, dialkylaminosulfonyl, alkoxycarbonyl, acyl, benzoyl, cycloalkylcarbonyl, optionally substituted C₁-C₁₀ alkyl, optionally substituted C₅-C₁₀ aryl optionally substituted C₃-C₁₀ heteroaryl, optionally substituted C₃-C₁₀ cycloalkyl or optionally substituted C₃-C₁₀ heterocycloalkyl. In some embodiments R^{7A} is sulfonyl, dialkylaminosulfonyl, alkoxycarbonyl, acyl, benzoyl, cycloalkylcarbonyl, C₅-C₁₀ aryl or optionally substituted C₃-C₁₀ heterocycloalkyl.

In some embodiments a compound for use in the invention comprises a compound of Formula IA wherein R^{2A} is

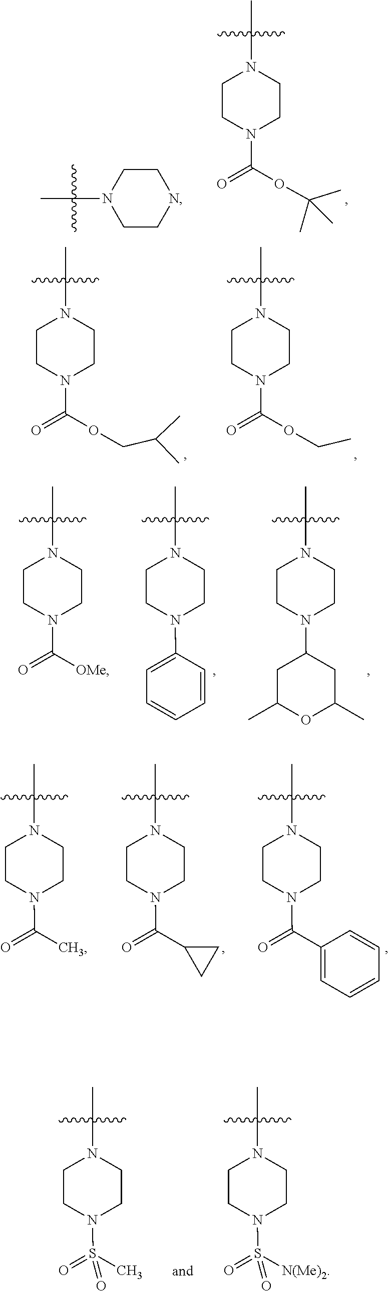

In various embodiments, a compound for use in the invention comprises a compound of Formula IA wherein R^{2A} is optionally substituted pyrrolidinyl:

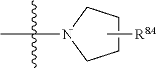

where R^{8A} is hydrogen, hydroxyl, sulfonyl, optionally substituted C₁-C₁₀ alkyl, optionally substituted C₅-C₁₀ aryl, optionally substituted C₃-C₁₀ heteroaryl, optionally substituted C₃-C₁₀ cycloalkyl or optionally substituted C₃-C₁₀ heterocycloalkyl. In some embodiments, R^{8A} is hydrogen, hydroxyl or optionally substituted C₁-C₁₀ alkyl.

In some embodiments, a compound for use in the invention comprises a compound of Formula IA wherein R_{2A} is:

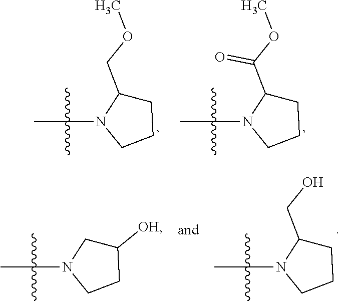

In some embodiments, a compound for use in the invention comprises a compound of Formula IA wherein R^{2A} is an optionally substituted bicyclic ring or an optionally substituted fused ring. For example, in some embodiments, R^{2A} is selected from the group consisting of:

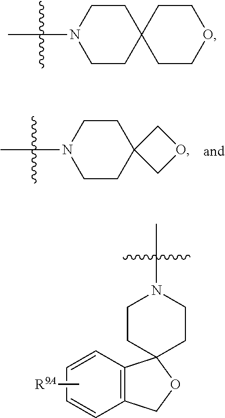

where R^{9A} is hydrogen, hydroxyl, sulfonyl, optionally substituted C₁-C₁₀ alkyl, optionally substituted C₅-C₁₀ aryl, optionally substituted C₃-C₁₀ heteroaryl, optionally substituted C₃-C₁₀cycloalkyl or optionally substituted C₃-C₁₀ heterocycloalkyl.

In some embodiments, a compound for use in the invention comprises a compound of Formula IA wherein R^{2A} is

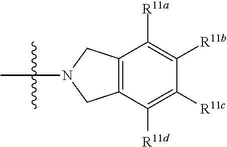

wherein each of R^{11a}, R^{11b}, R^{11c}, and R^{11d}, is, independently selected from, hydrogen, hydroxy, sulfonyl, optionally substituted C₁-C₁₀ alkyl, optionally substituted C₅-C₁₀ aryl, optionally substituted C₃-C₁₀ heteroaryl, optionally substituted C₃-C₁₀ cycloalkyl or optionally substituted C₃-C₁₀ heterocycloalkyl. In particular embodiments, R^{2A} is

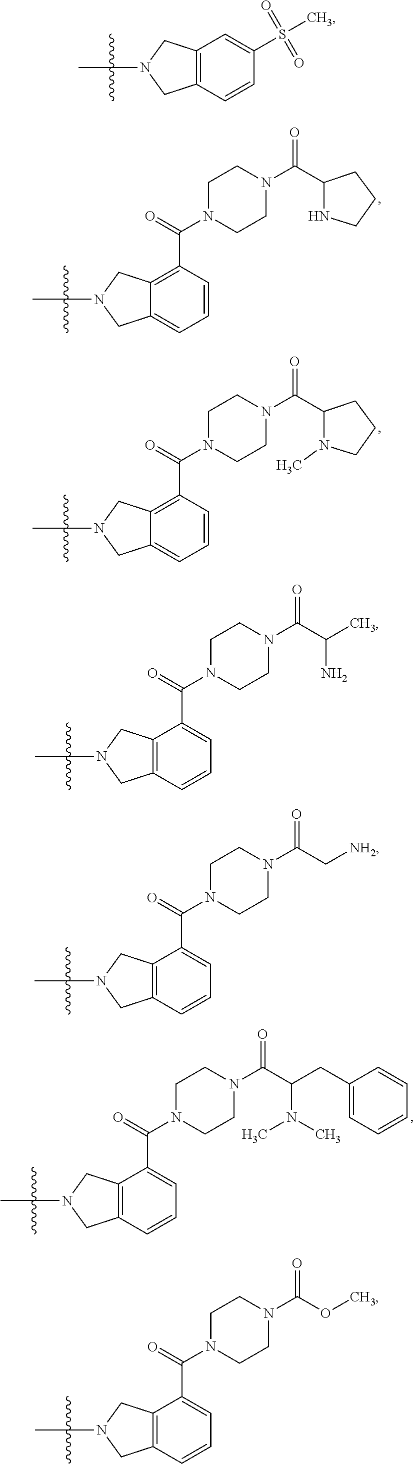

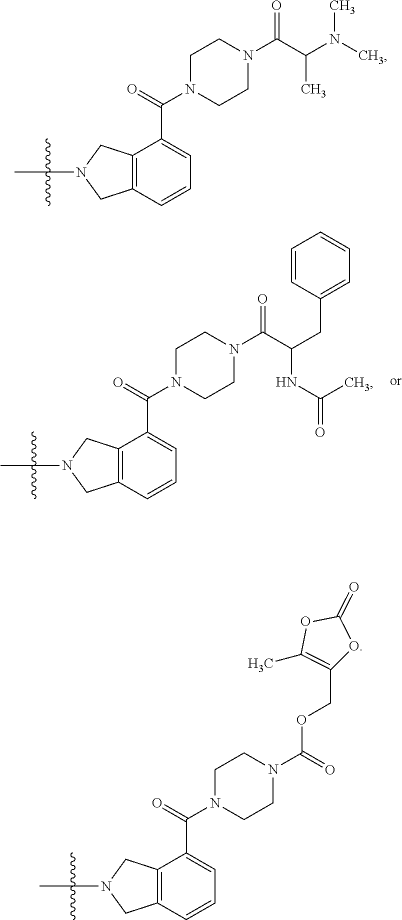 [SMILES: *N1Cc2cccc(C(=O)N3CCN(C(=O)C(C)N(C)C)CC3)c2C1 ; *N1Cc2cccc(C(=O)N3CCN(C(=O)C(Cc4ccccc4)NC(C)=O)CC3)c2C1 ; "or" ; *N1Cc2cccc(C(=O)N3CCN(C(=O)OCc4oc(=O)oc4C)CC3)c2C1]

In some embodiments a compound for use in the invention is a compound of Formula IA wherein each R^{a}, R^{b}, R^{c}, R^{d} and R^{e} is selected from any embodiment disclosed herein for each of R^{a}, R^{b}, R^{c}, R^{d} and R^{e}; R^{1A} is selected from any embodiment disclosed herein for R^{1A}; and R^{2A} is selected from any embodiment disclosed herein for R^{2A}.

In some embodiments a compound for use in the invention is a compound selected from the group consisting of:

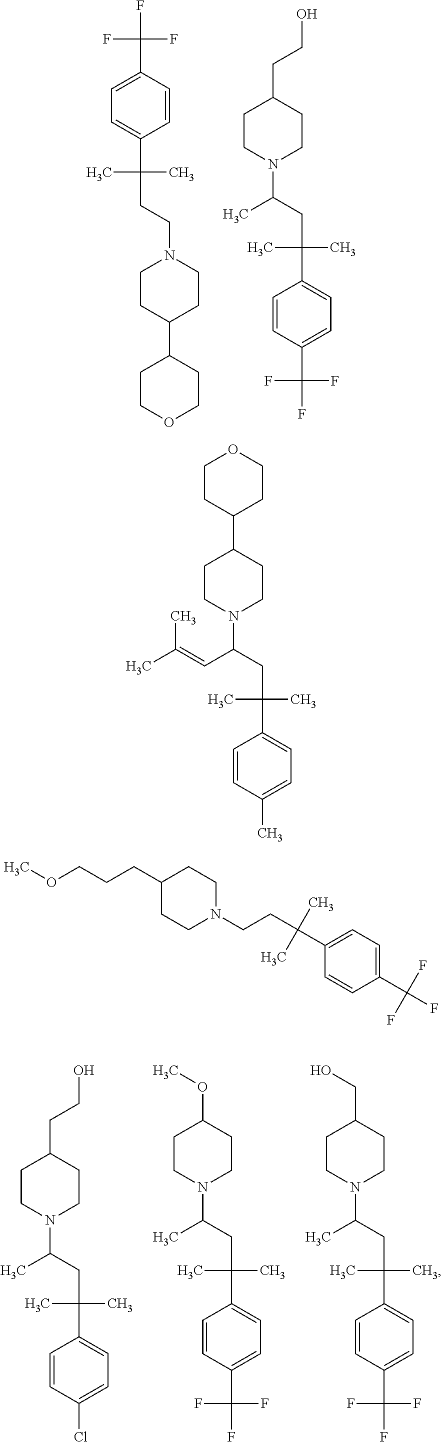

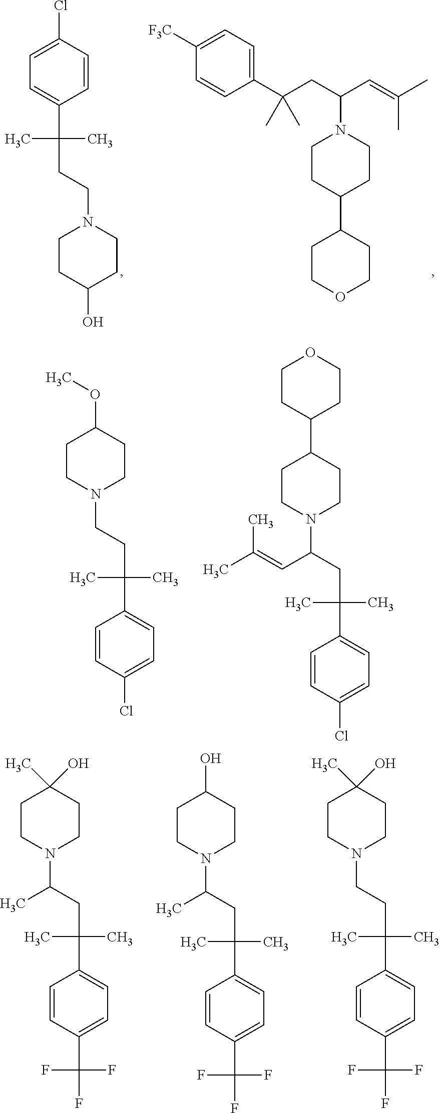

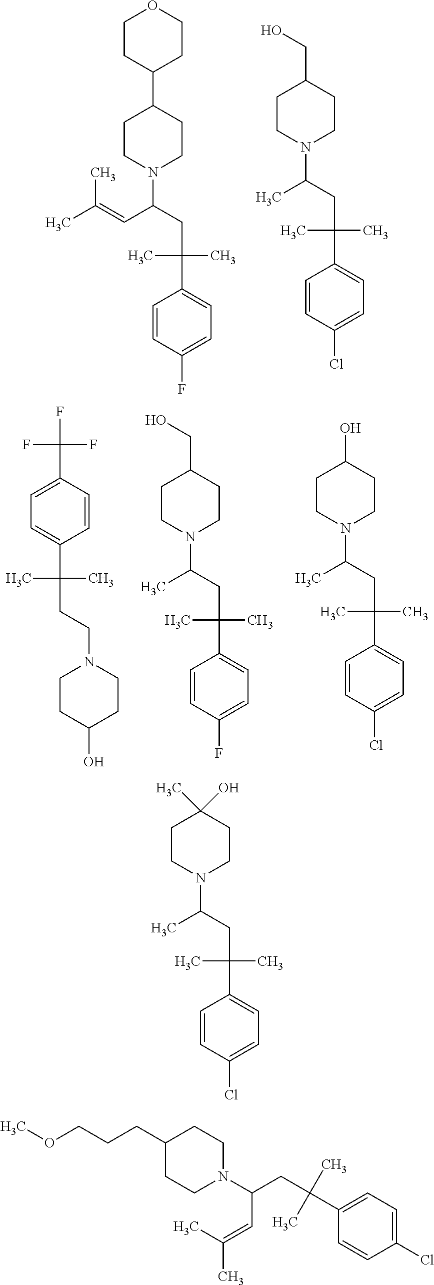

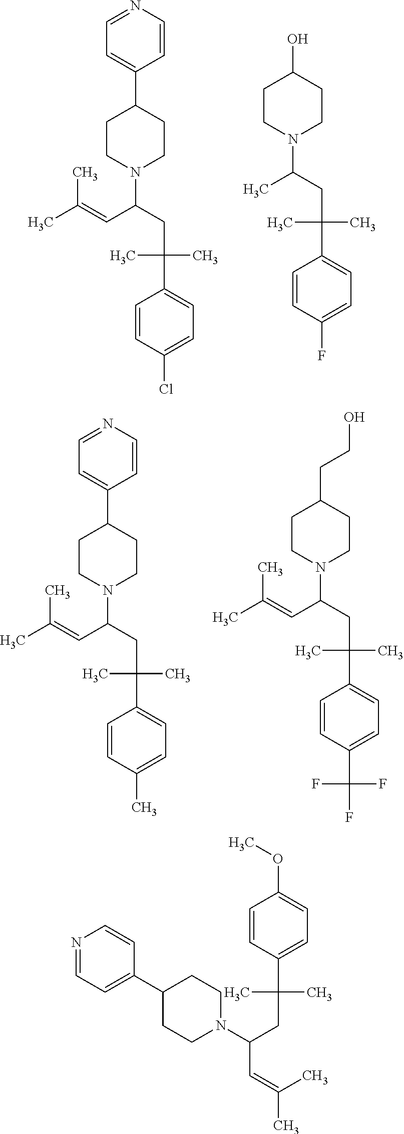 [SMILES: CC(C)=CC(CC(C)(C)c1ccc(Cl)cc1)N1CCC(c2ccncc2)CC1 ; CC(CC(C)(C)c1ccc(F)cc1)N1CCC(O)CC1 ; CC(C)=CC(CC(C)(C)c1ccc(C)cc1)N1CCC(c2ccncc2)CC1 ; CC(C)=CC(CC(C)(C)c1ccc(C(F)(F)F)cc1)N1CCC(CCO)CC1 ; COc1ccc(C(C)(C)CC(C=C(C)C)N2CCC(c3ccncc3)CC2)cc1]

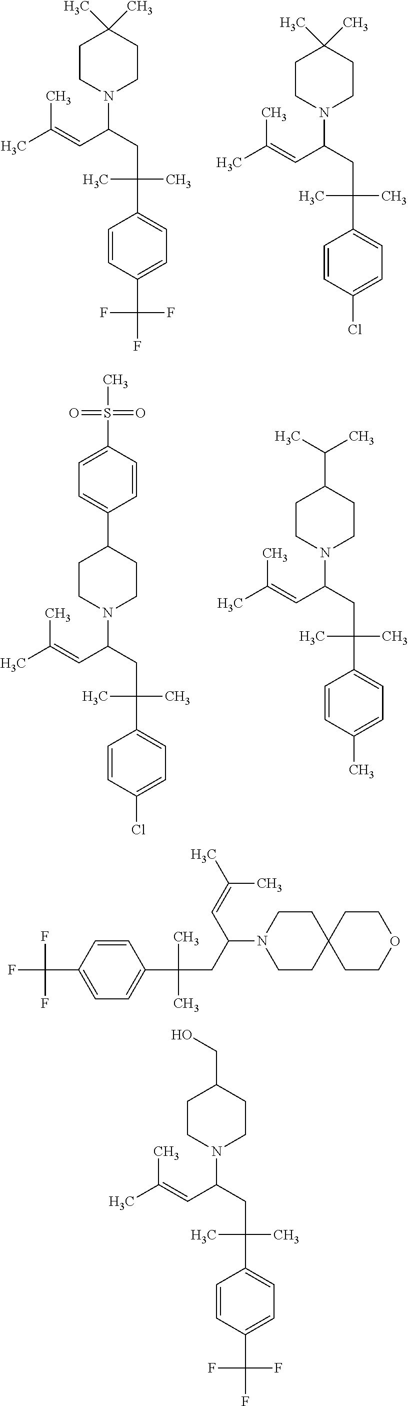

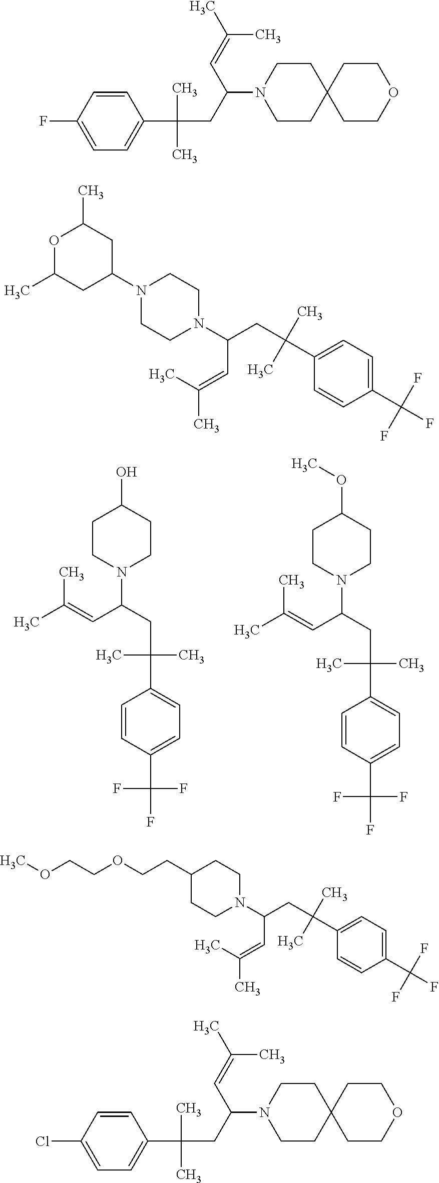

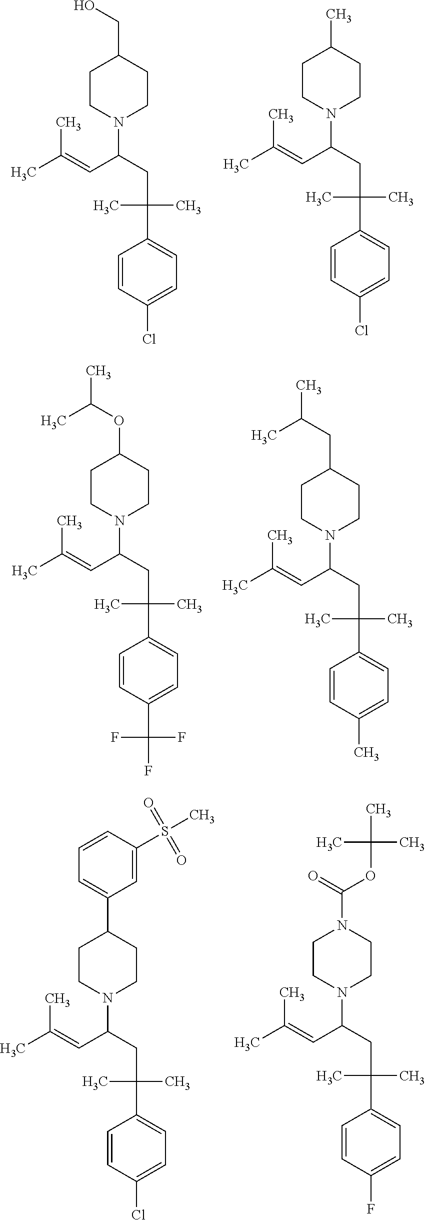

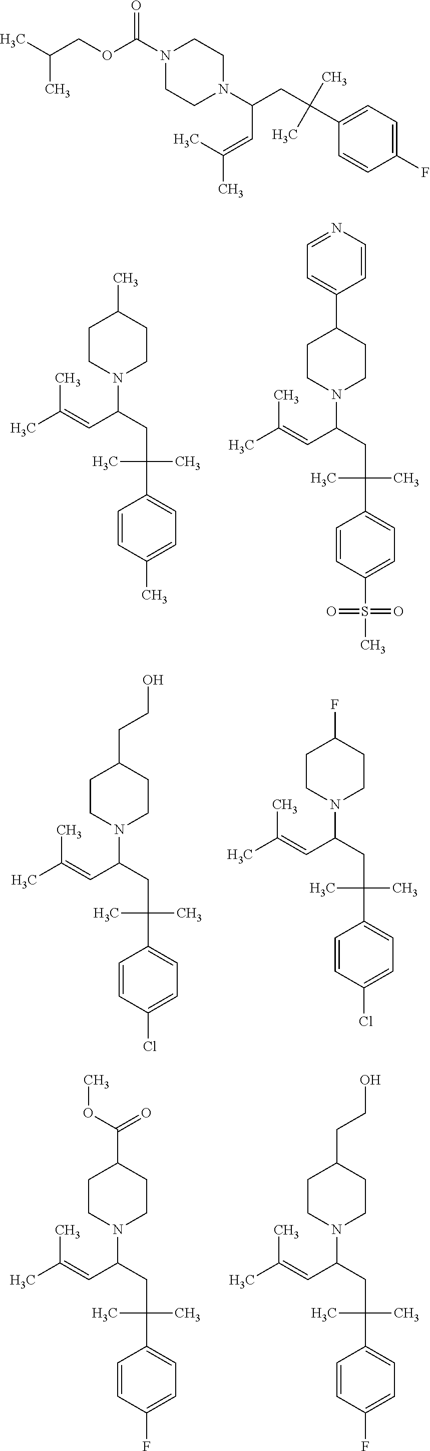

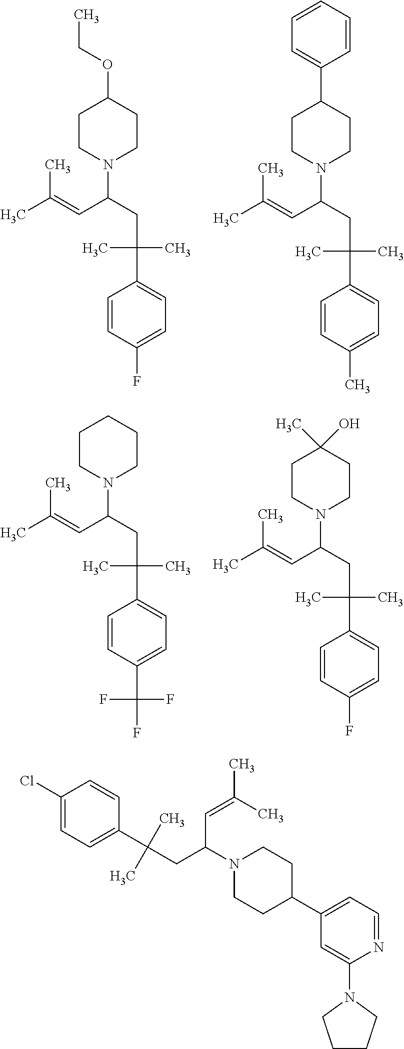

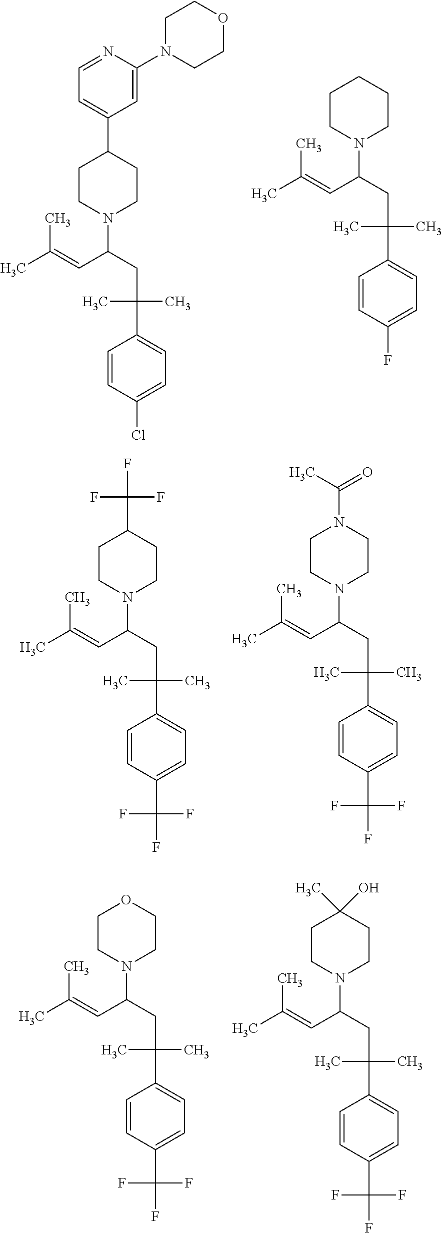

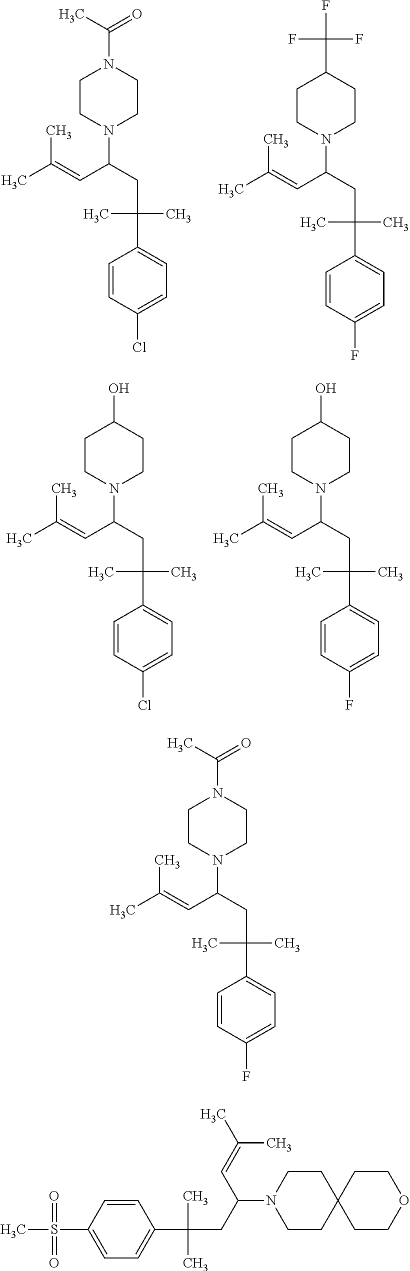

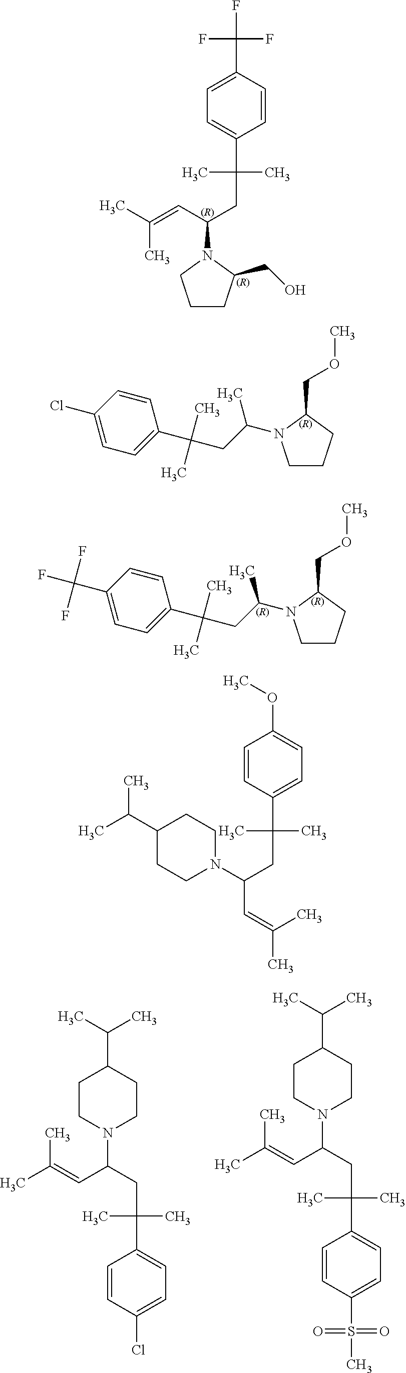

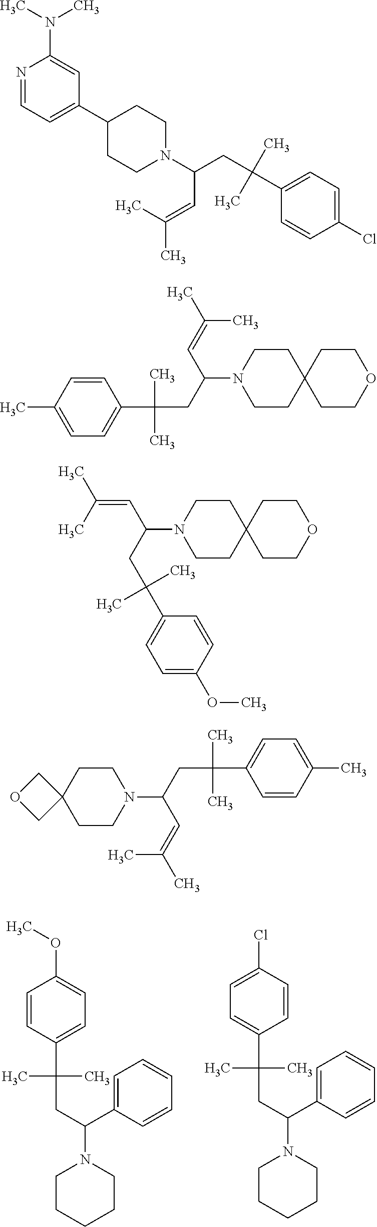

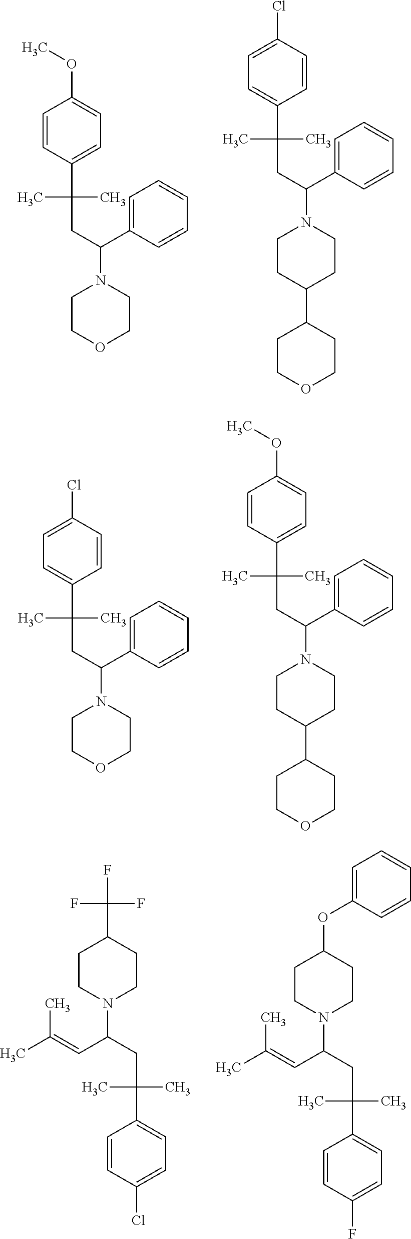 [SMILES: COc1ccc(C(C)(C)CC(c2ccccc2)N2CCOCC2)cc1 ; CC(C)(CC(c1ccccc1)N1CCC(C2CCOCC2)CC1)c1ccc(Cl)cc1 ; CC(C)(CC(c1ccccc1)N1CCOCC1)c1ccc(Cl)cc1 ; COc1ccc(C(C)(C)CC(c2ccccc2)N2CCC(C3CCOCC3)CC2)cc1 ; CC(C)=CC(CC(C)(C)c1ccc(Cl)cc1)N1CCC(C(F)(F)F)CC1 ; CC(C)=CC(CC(C)(C)c1ccc(F)cc1)N1CCC(Oc2ccccc2)CC1]

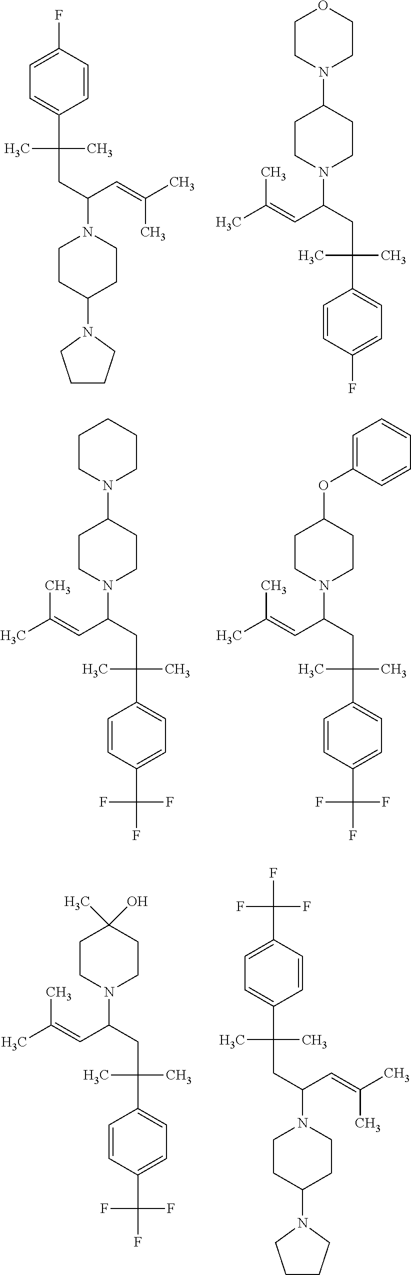

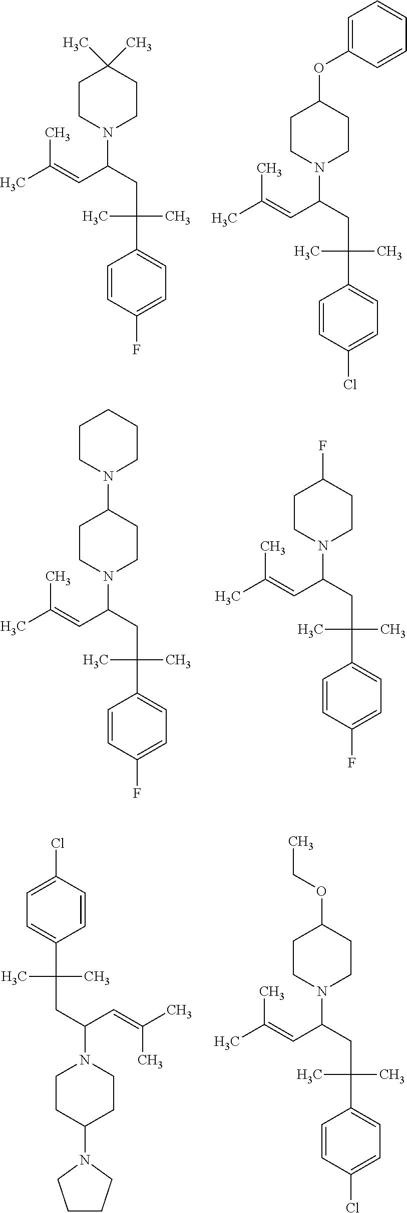 [SMILES: CC(C)=CC(CC(C)(C)c1ccc(F)cc1)N1CCC(C)(C)CC1 ; CC(C)=CC(CC(C)(C)c1ccc(Cl)cc1)N1CCC(Oc2ccccc2)CC1 ; CC(C)=CC(CC(C)(C)c1ccc(F)cc1)N1CCC(N2CCCCC2)CC1 ; CC(C)=CC(CC(C)(C)c1ccc(F)cc1)N1CCC(F)CC1 ; CC(C)=CC(CC(C)(C)c1ccc(Cl)cc1)N1CCC(N2CCCC2)CC1 ; CCOC1CCN(C(C=C(C)C)CC(C)(C)c2ccc(Cl)cc2)CC1]

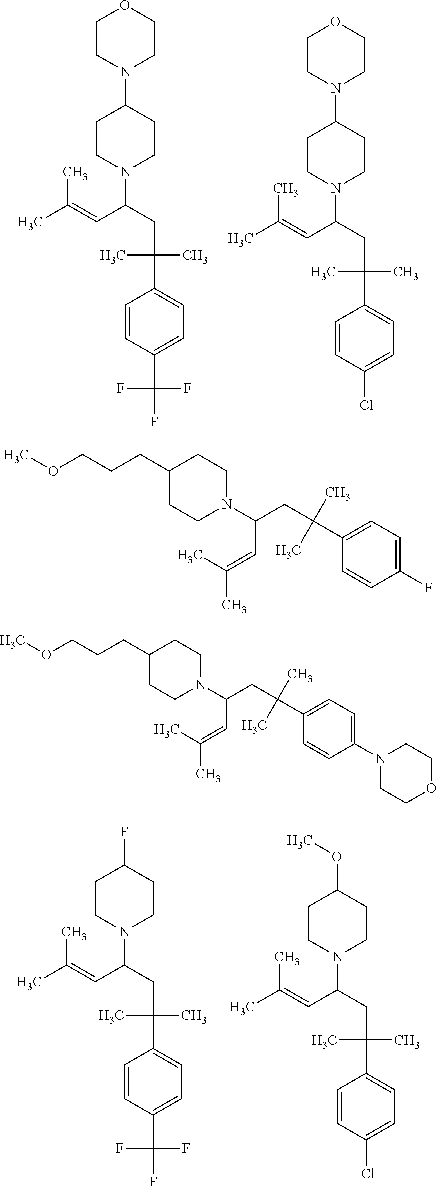

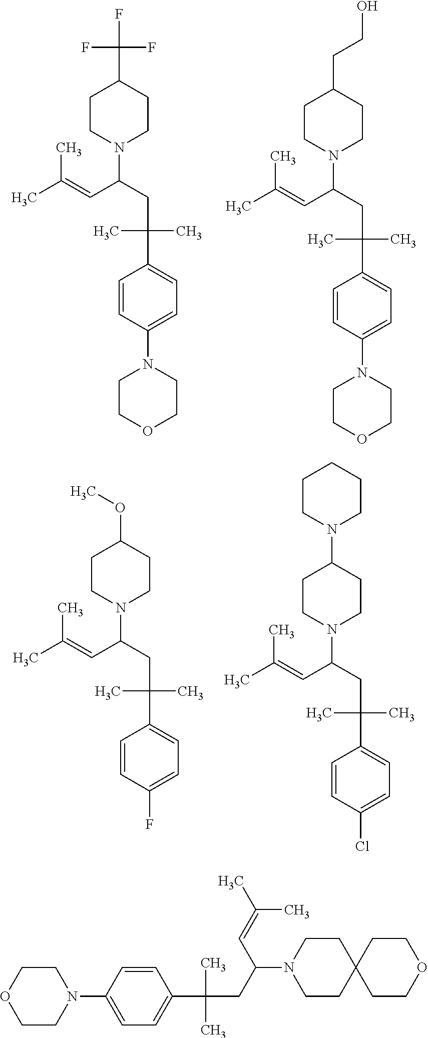

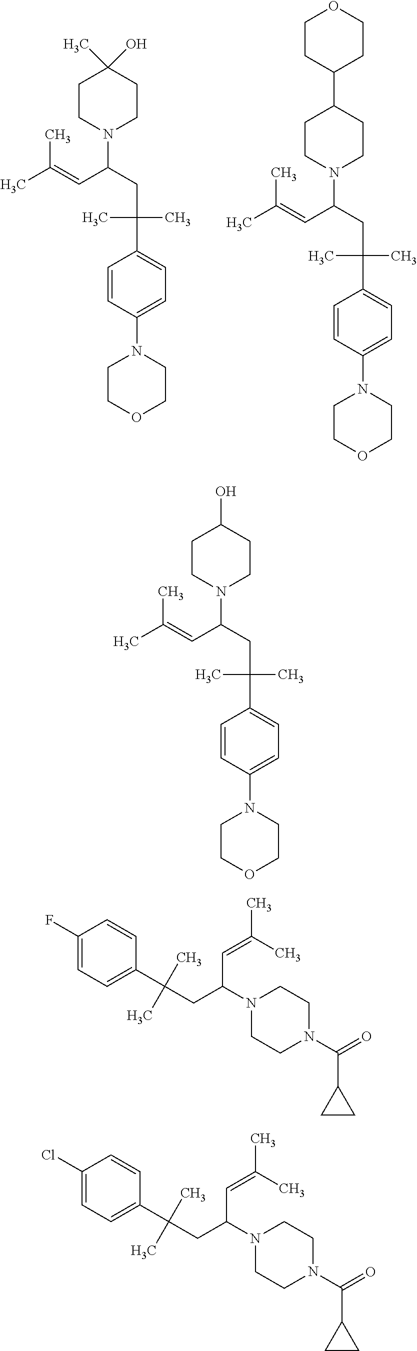

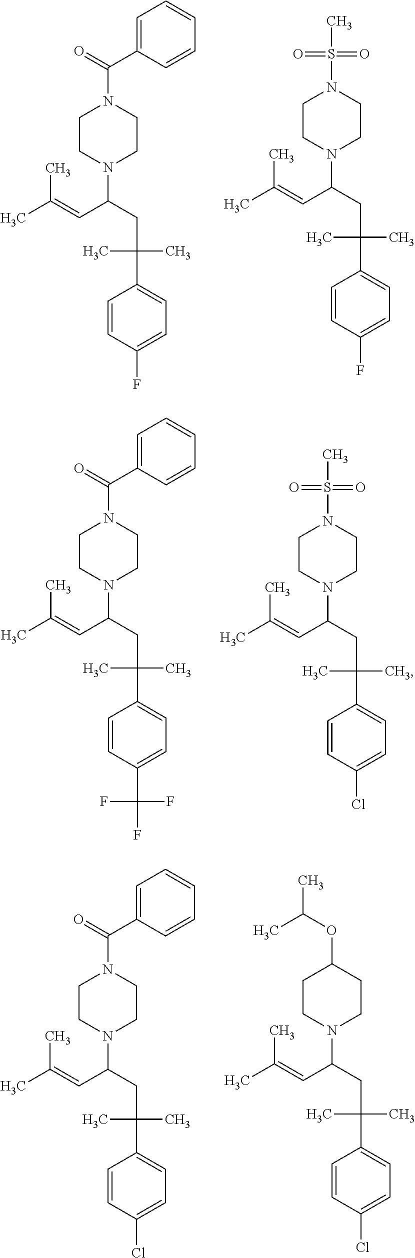

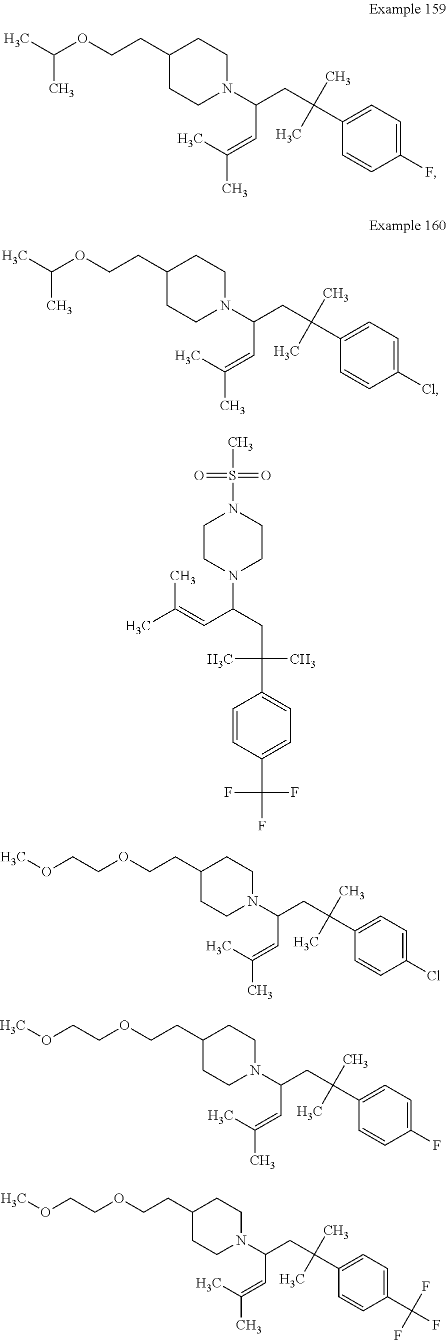

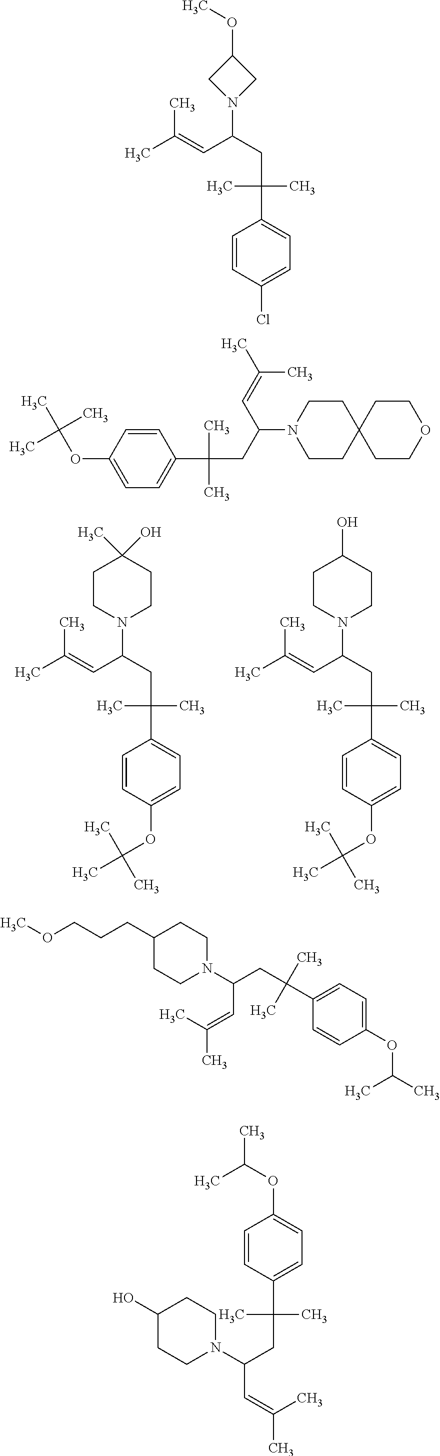

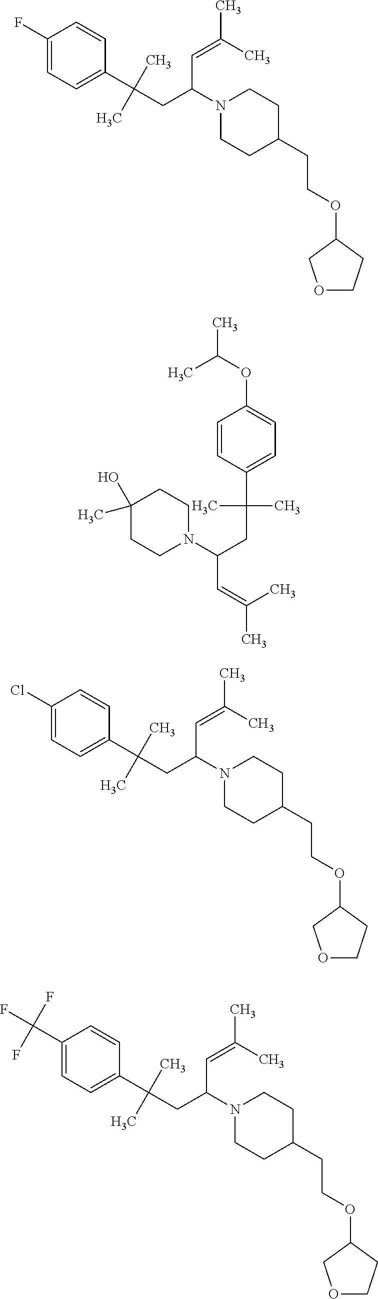

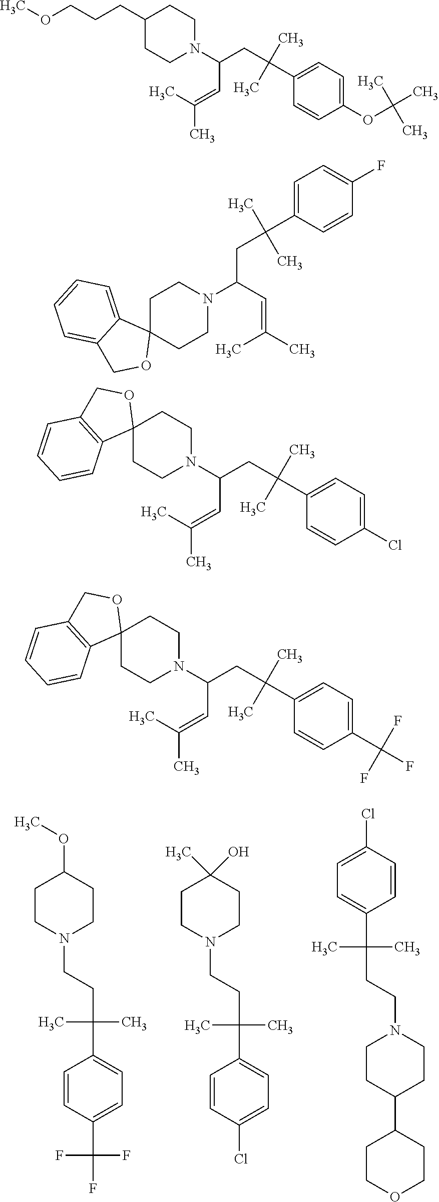

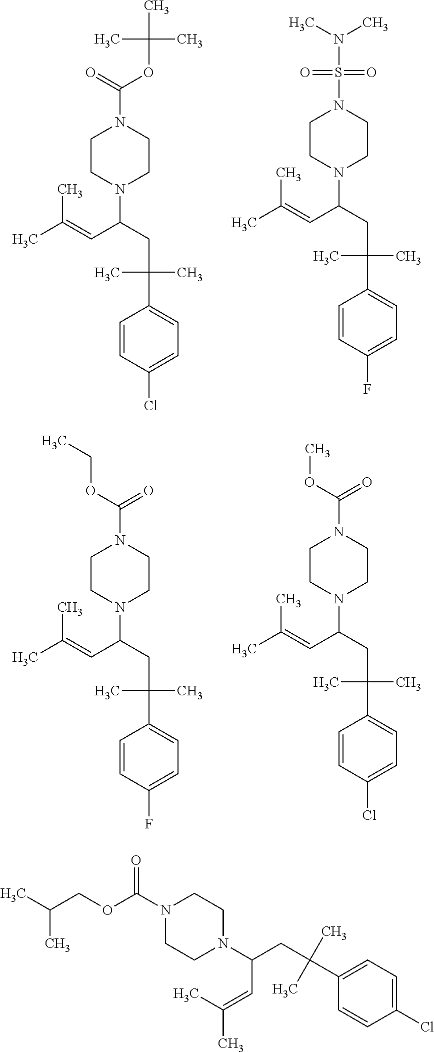

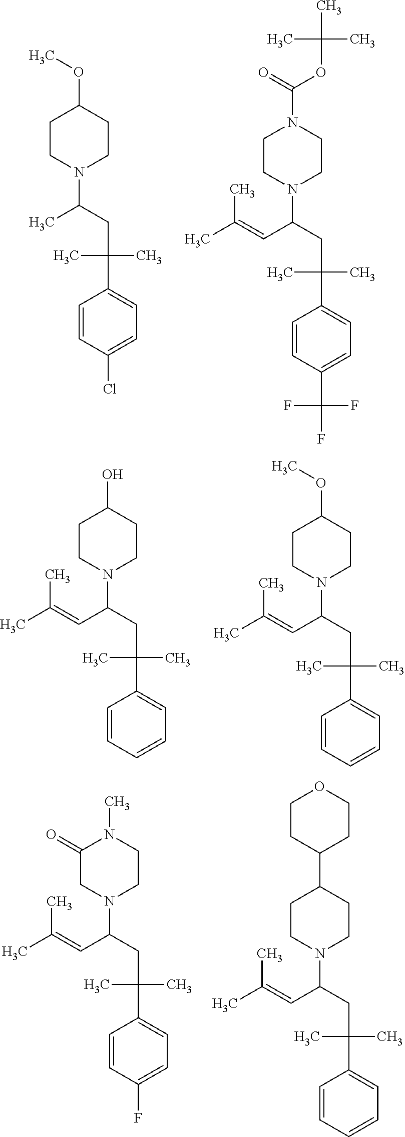

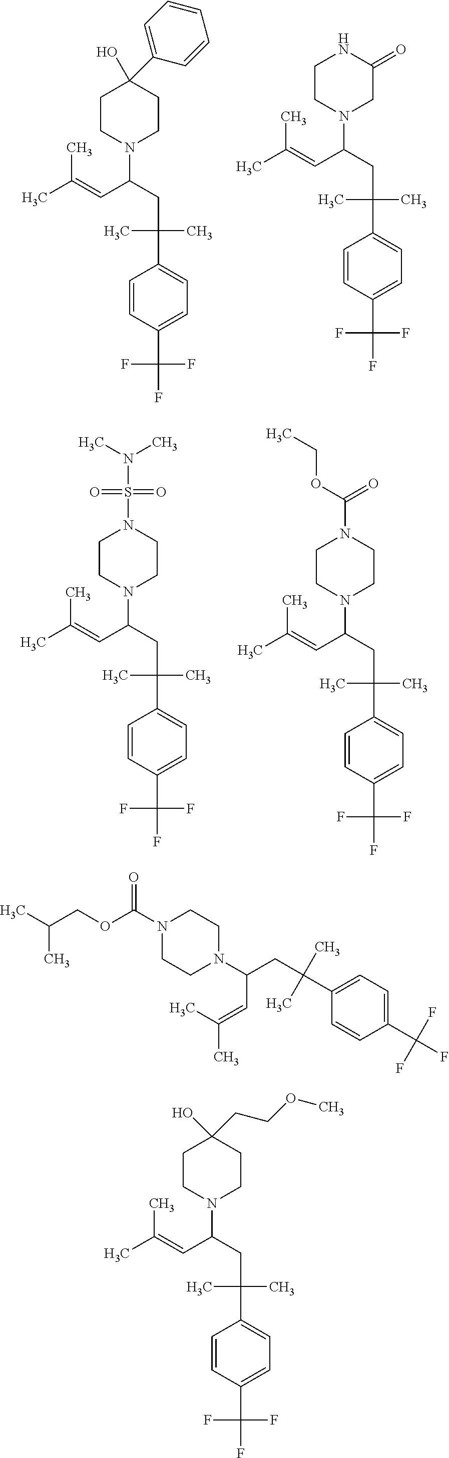

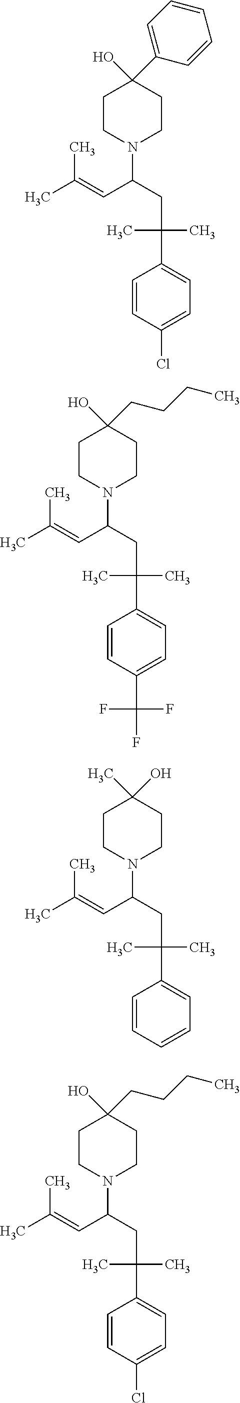

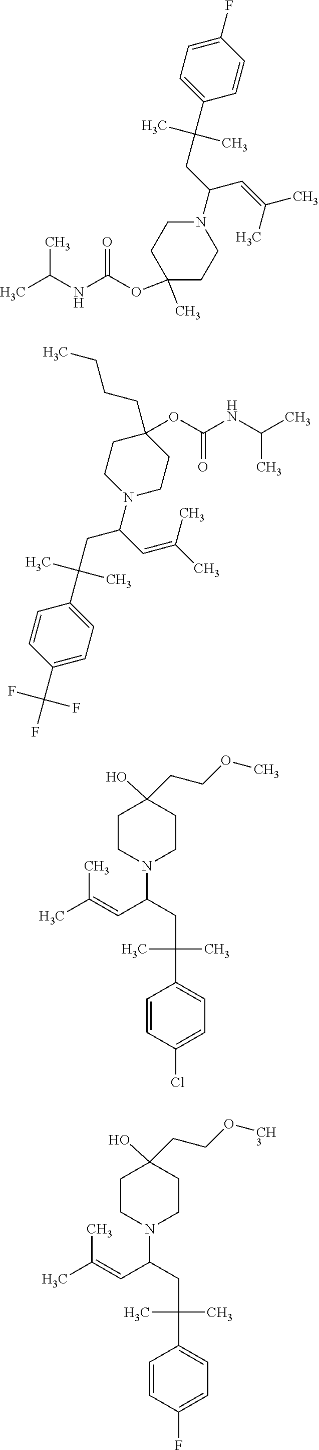

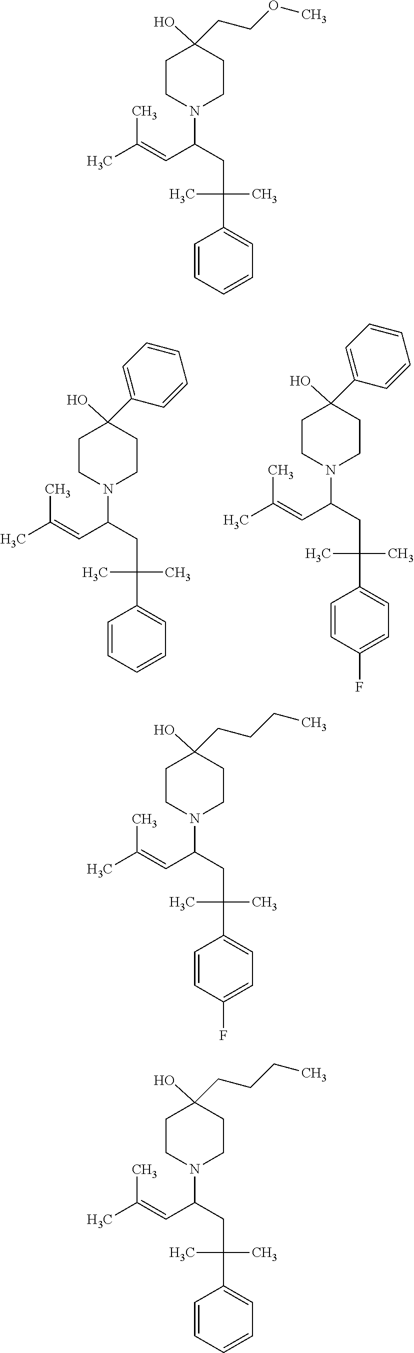

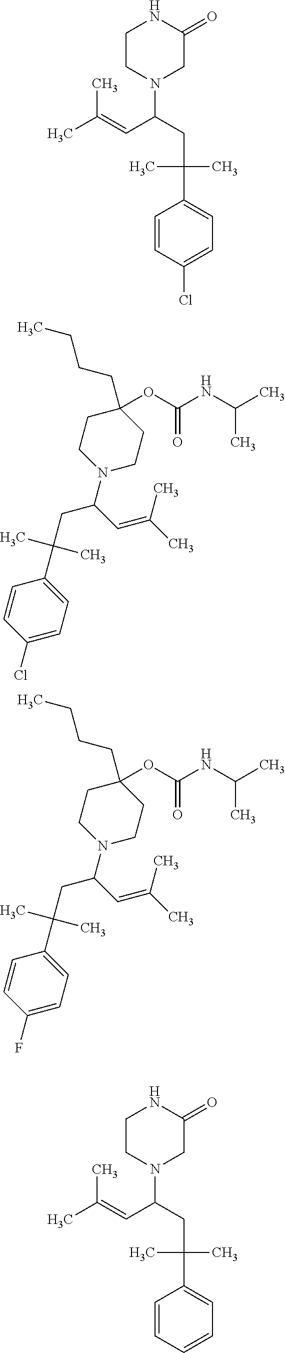

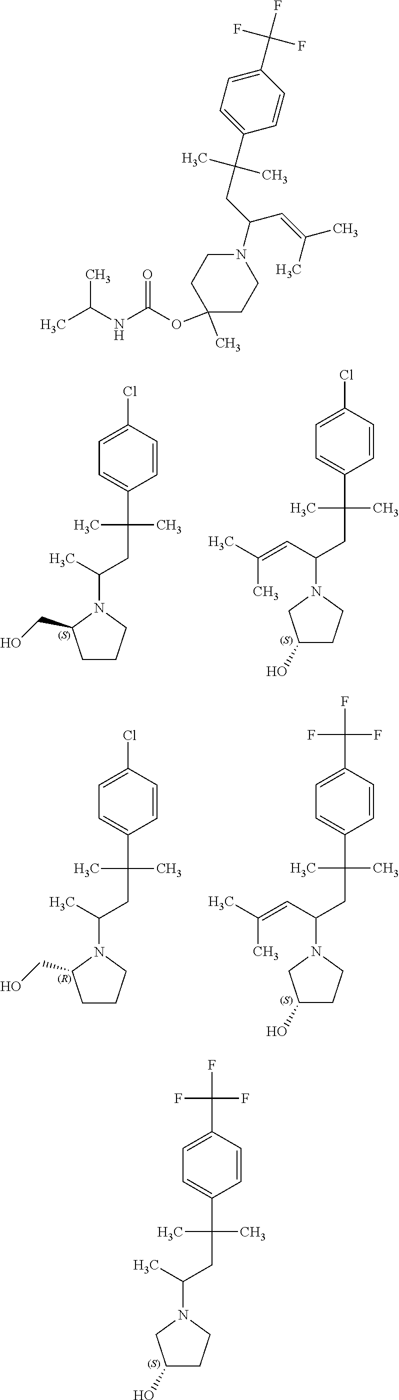

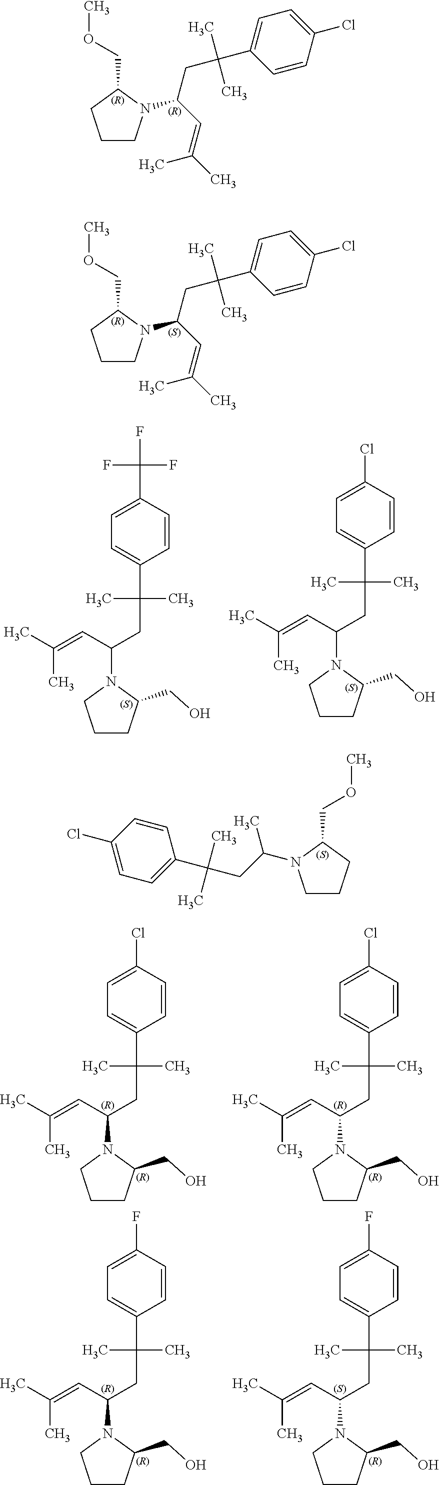

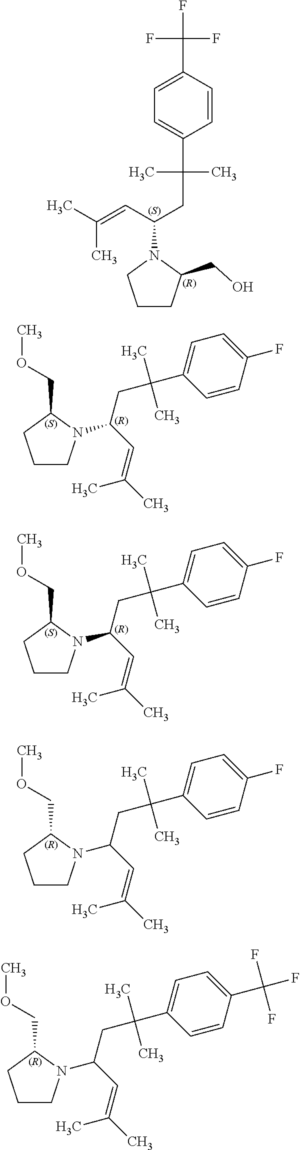

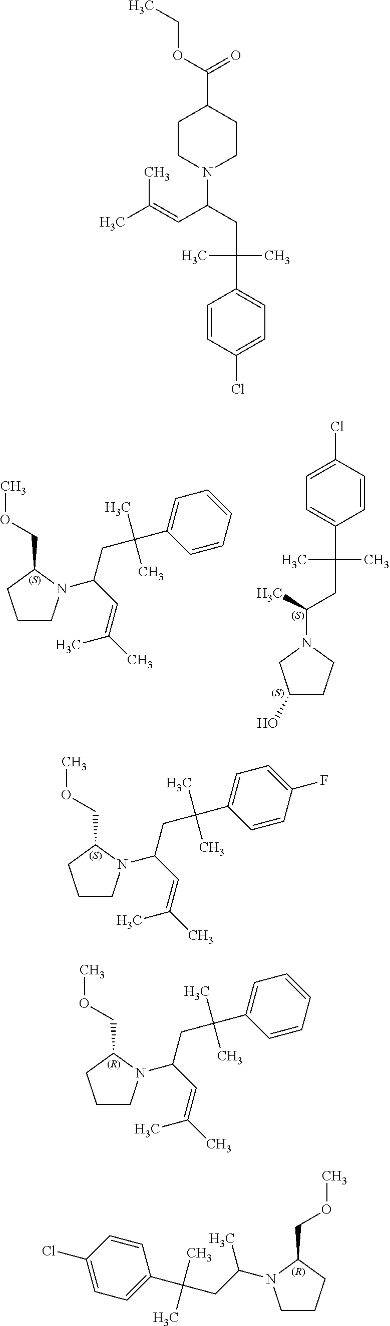

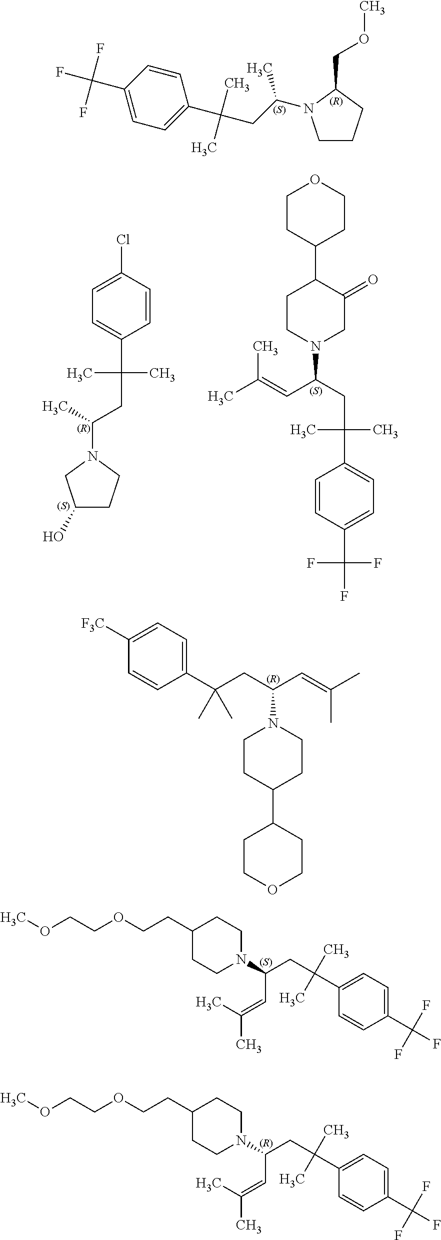

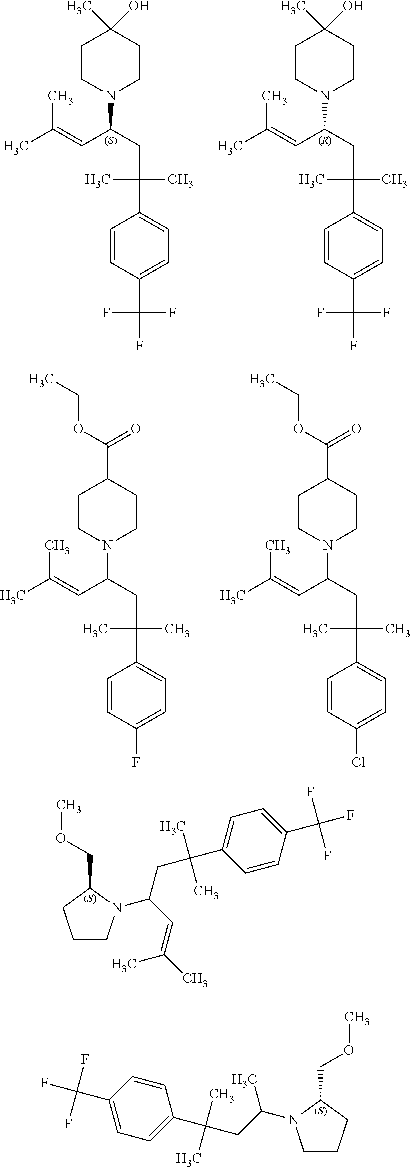

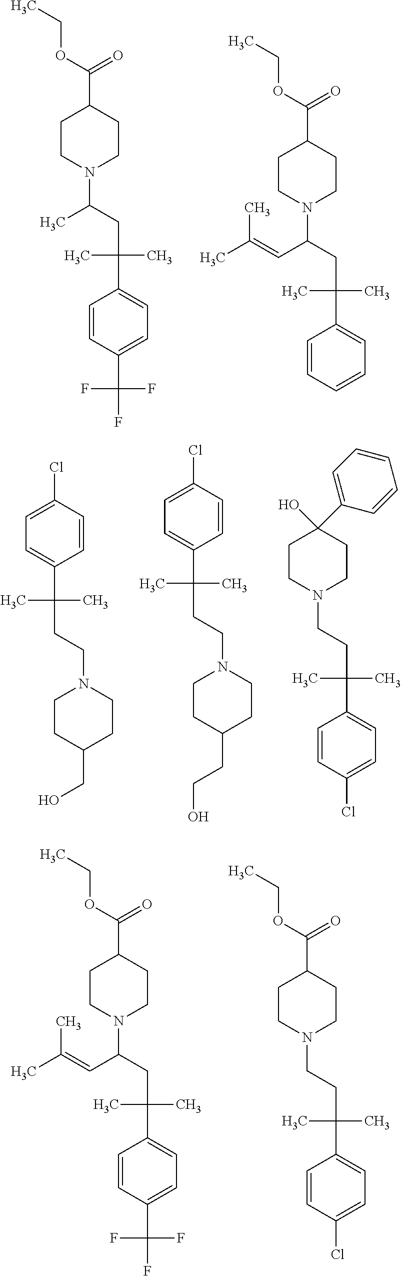

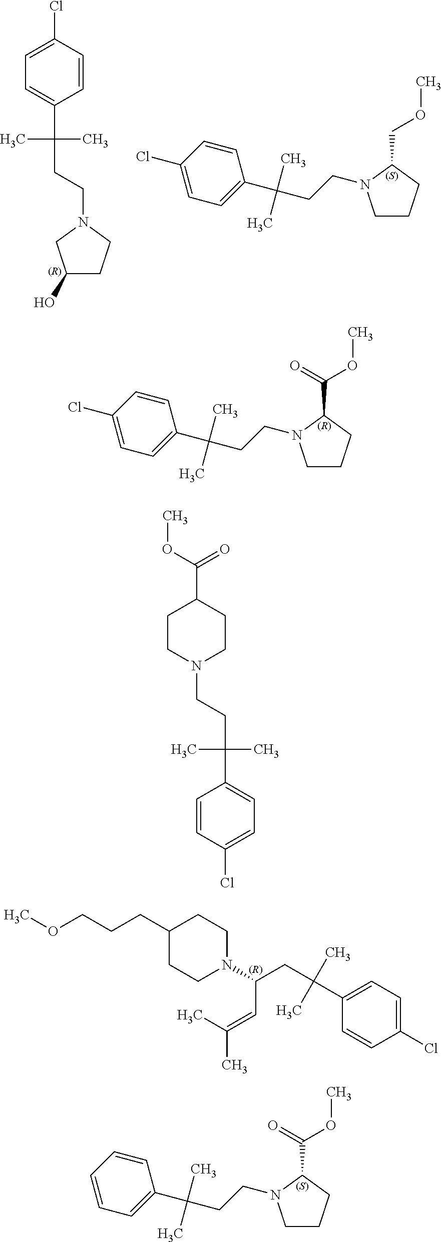

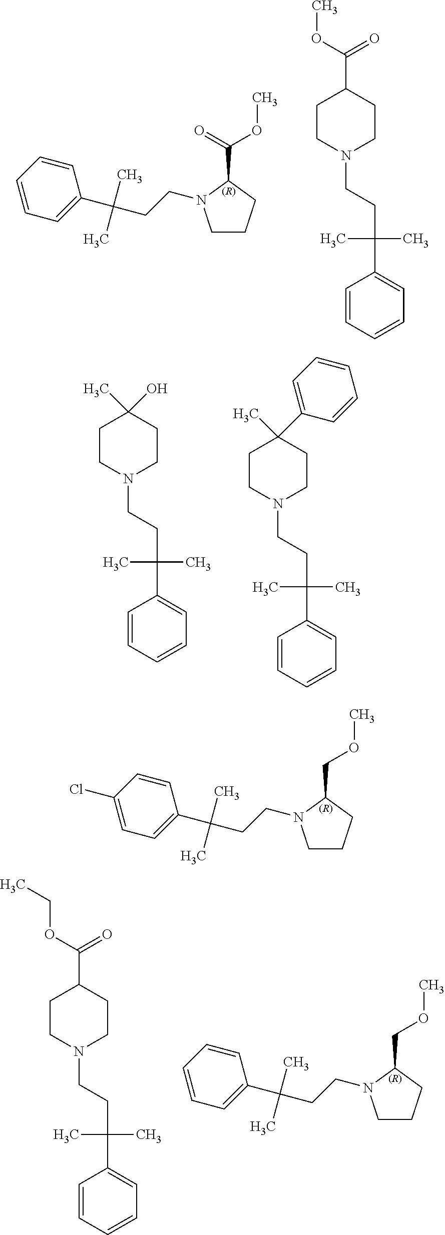

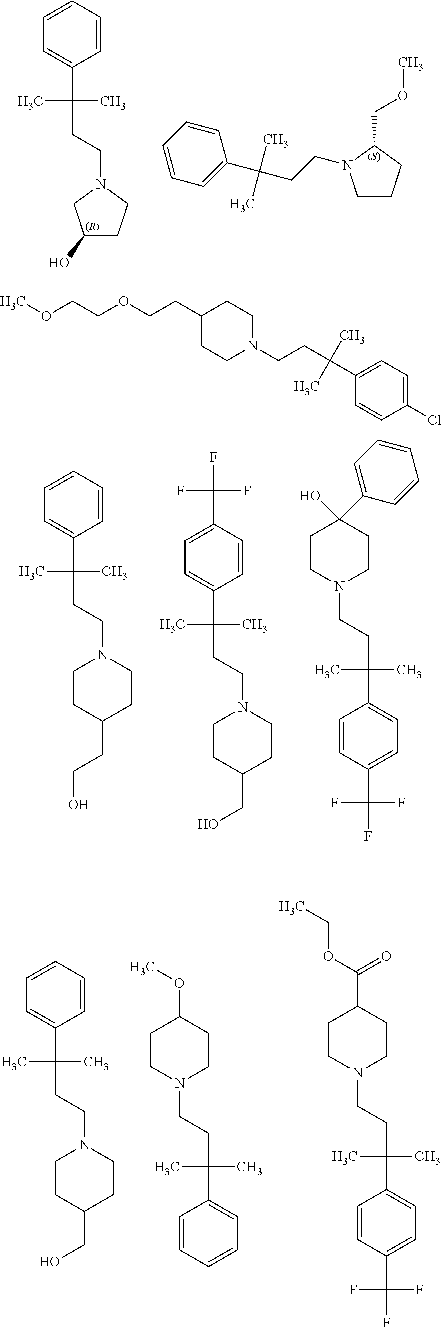

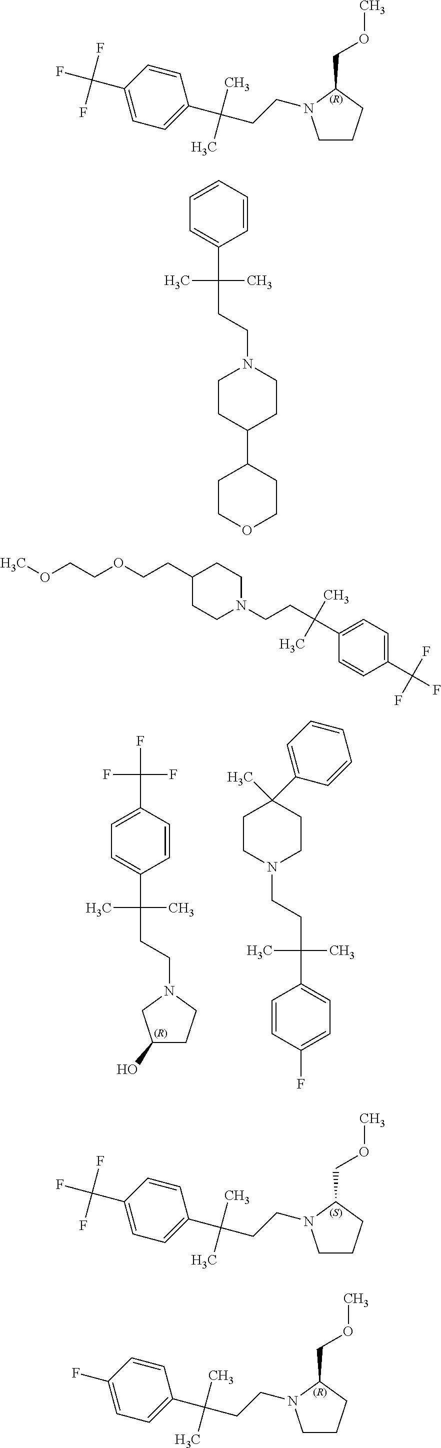

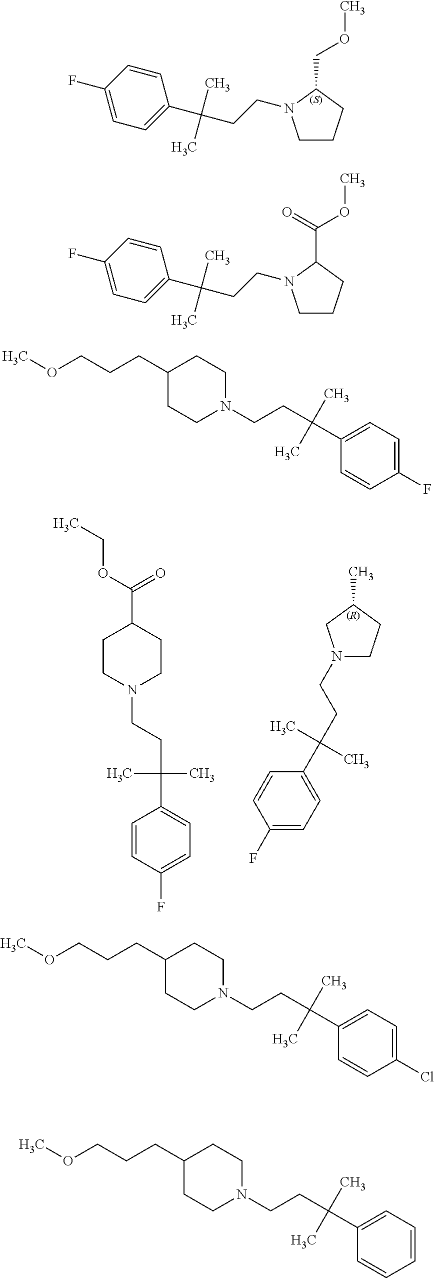

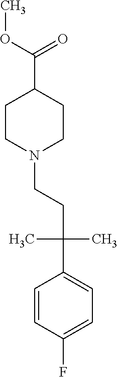 [SMILES: COC(=O)C1CCN(CCC(C)(C)c2ccc(F)cc2)CC1]

In some embodiments a compound for use in the invention is a compound selected from the group consisting of:

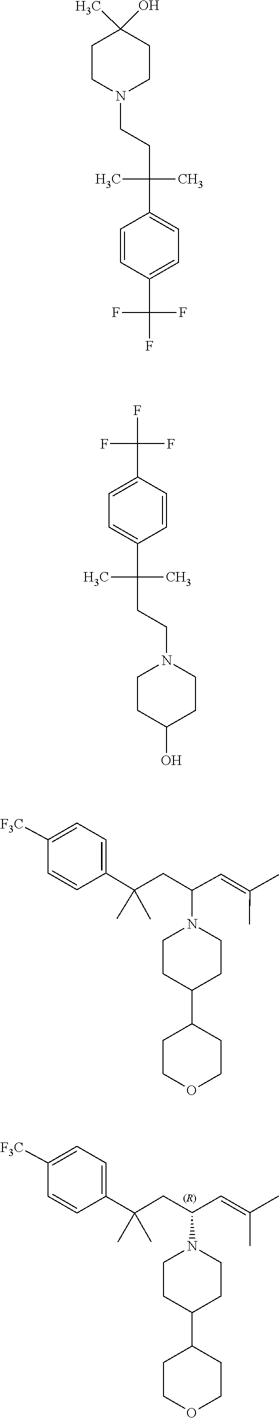

In some embodiments a compound for use in the invention is a compound of Formula IIA or pharmaceutically acceptable salt thereof:

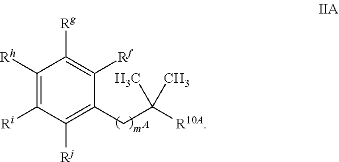

Each of substituents R^{f}, R^{g}, R^{h}, Rⁱ and R^{j} of Formula IIA is independently selected from the group consisting of, H, hydroxyl, halo, alkyl, alkoxy, CF₃, SO₂CH₃, and morpholino.

Substituent R^{10A} of Formula IIA is an optionally substituted cyclic amino group and m^{A} is an integer from 0 to 3.

In some embodiments each of substituents R^{f}, R^{g}, R^{h}, Rⁱ and R^{j} of Formula IIA is independently selected from the group consisting of, H, hydroxyl, and alkoxy. In some embodiments each of substituents R^{f}, R^{g}, R^{h}, Rⁱ and R^{j} of Formula IIA is independently selected from the group consisting of, H, hydroxyl, and methoxy. In some embodiments each of substituents R^{f}, R^{g}, and R^{j} is H and each of R^{g}, and R^{h} is independently selected from the hydroxyl, or methoxy.

In some embodiments, R^{10A} of Formula IIA is an optionally substituted aziridinyl, optionally substituted pyrolidinyl, optionally substituted imidizolidinyl, optionally substituted piperidinyl, optionally substituted piperazinyl, optionally substituted oxopiperazinyl, or optionally substituted morpholinyl, and any of the individual substituted or unsubstituted piperdinyl, substituted or unsubstituted morpholinyl, substituted or unsubstituted piperazinyl, substituted or unsubstituted pyrrolidinyl, substituted or unsubstituted bicyclic, or substituted or unsubstituted fused rings described above in relation to Formula I.

In some embodiments, R^{10A} of Formula IIA is an optionally substituted fused ring, such as:

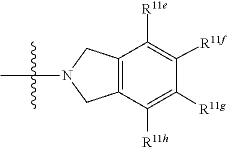

wherein each of R^{11e}, R^{11f}, R^{11g}, and R^{11h} is independently selected from, hydrogen, hydroxy, sulfonyl, optionally substituted C₁-C₁₀ alkyl, optionally substituted C₅-C₁₀ aryl optionally substituted C₃-C₁₀ heteroaryl, optionally substituted C₃-C₁₀ cycloalkyl or optionally substituted C₃-C₁₀ heterocycloalkyl. In certain embodiments R^{10A} is not

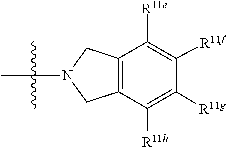

when m^{A} is 2.

In some embodiments, R^{10A} f Formula IIA is

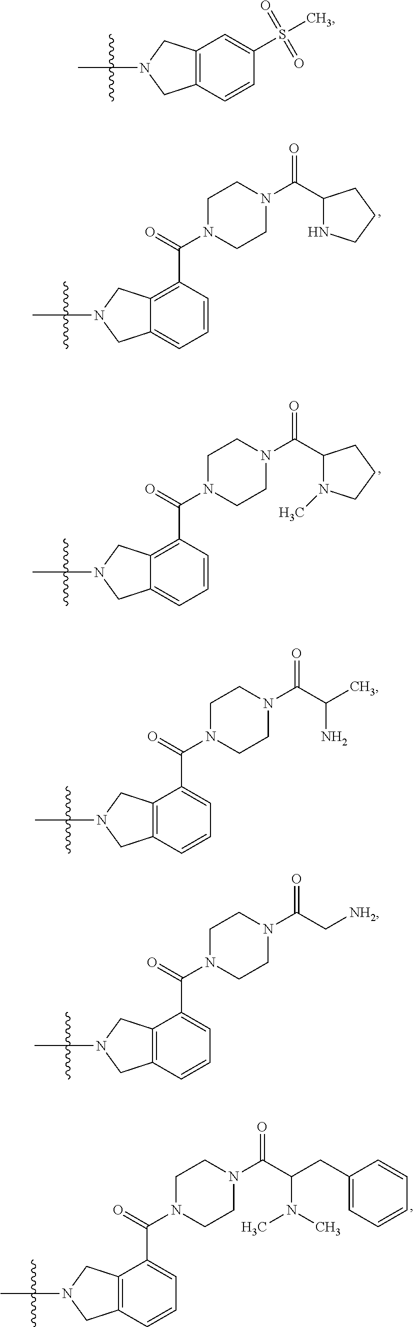 [SMILES: *N1Cc2ccc(S(C)(=O)=O)cc2C1 ; *N1Cc2cccc(C(=O)N3CCN(C(=O)C4CCCN4)CC3)c2C1 ; *N1Cc2cccc(C(=O)N3CCN(C(=O)C4CCCN4C)CC3)c2C1 ; *N1Cc2cccc(C(=O)N3CCN(C(=O)C(C)N)CC3)c2C1 ; *N1Cc2cccc(C(=O)N3CCN(C(=O)CN)CC3)c2C1 ; *N1Cc2cccc(C(=O)N3CCN(C(=O)C(Cc4ccccc4)N(C)C)CC3)c2C1]

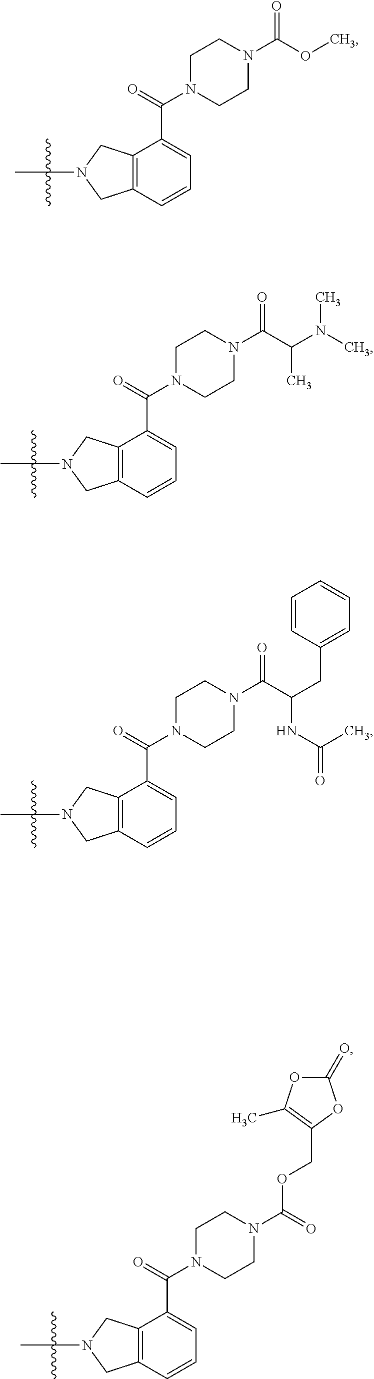 [SMILES: *N1Cc2cccc(C(=O)N3CCN(C(=O)OC)CC3)c2C1 ; *N1Cc2cccc(C(=O)N3CCN(C(=O)C(C)N(C)C)CC3)c2C1 ; *N1Cc2cccc(C(=O)N3CCN(C(=O)C(Cc4ccccc4)NC(C)=O)CC3)c2C1 ; *N1Cc2cccc(C(=O)N3CCN(C(=O)OCc4oc(=O)oc4C)CC3)c2C1]

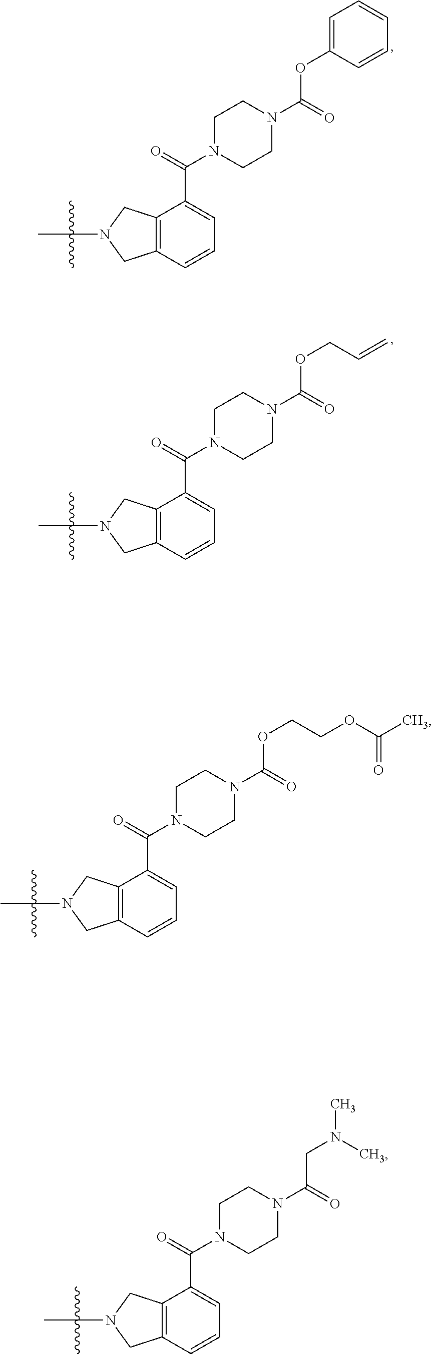 [SMILES: *N1Cc2cccc(C(=O)N3CCN(C(=O)Oc4ccccc4)CC3)c2C1 ; *N1Cc2cccc(C(=O)N3CCN(C(=O)OCC=C)CC3)c2C1 ; *N1Cc2cccc(C(=O)N3CCN(C(=O)OCCOC(C)=O)CC3)c2C1 ; *N1Cc2cccc(C(=O)N3CCN(C(=O)CN(C)C)CC3)c2C1]

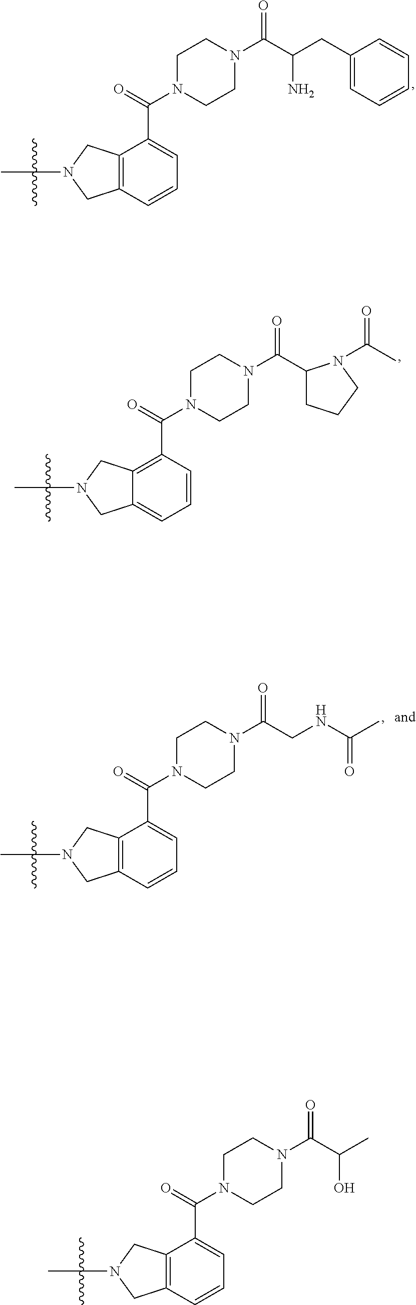 [SMILES: *N1Cc2cccc(C(=O)N3CCN(C(=O)C(N)Cc4ccccc4)CC3)c2C1 ; *N1Cc2cccc(C(=O)N3CCN(C(=O)C4CCCN4C(C)=O)CC3)c2C1 ; *N1Cc2cccc(C(=O)N3CCN(C(=O)CNC(C)=O)CC3)c2C1 ; *N1Cc2cccc(C(=O)N3CCN(C(=O)C(C)O)CC3)c2C1]

In some embodiments a compound for use in the invention is a compound of Formula IIa:

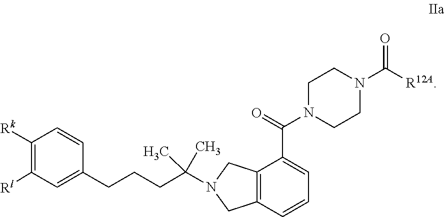

Each of substituents R^{k} and R^{l} of Formula IIa is independently selected from the group consisting of H, hydroxyl, halo, alkyl, alkoxy, CF₃, SO₂CH₃, and morpholino.

Substituent R^{12A} of Formula IIa is selected from the group consisting of aryloxy, alkenyloxy, alkoxy, aminoalkyl, N,N-dimethylaminoalkyl, pyrrolidinyl, n-methylpyrrolidinyl, N-acylpyrrolidinyl, carboxyaminoalkyl, hydroxyalkyl, —O(CH₂)₂OC(O)CH₃,

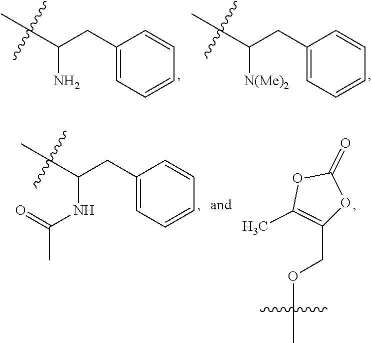

In some embodiments each of substituents R^{k} and R^{l} of Formula IIa is independently selected from the group consisting of H, hydroxyl and methoxy. In some embodiments R^{1A} is methoxy and R^{k} is hydroxyl.

In some embodiments substituent R^{12A} of Formula IIa is selected from the group consisting of phenyloxy, —OCH₂CH═CH₂, methoxy, —CH₂NH₂, —CH(NH₂)CH₃, —CH₂N(Me)₂, —CH(CH₃)N(Me)₂, —CH₂NHC(O)CH₃, —CH(OH)CH₃, —O(CH₂)₂OC(O)CH₃,

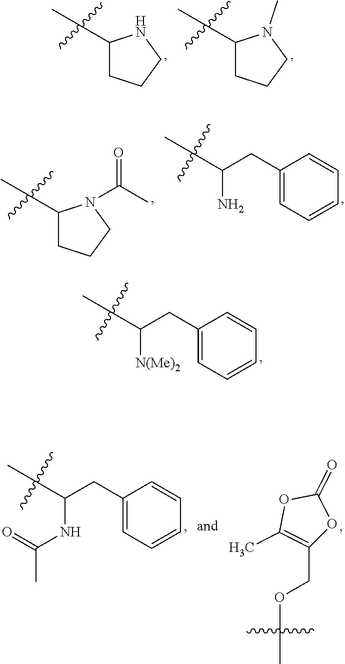

In some embodiments a compound for use in the invention is a compound selected from the group consisting of

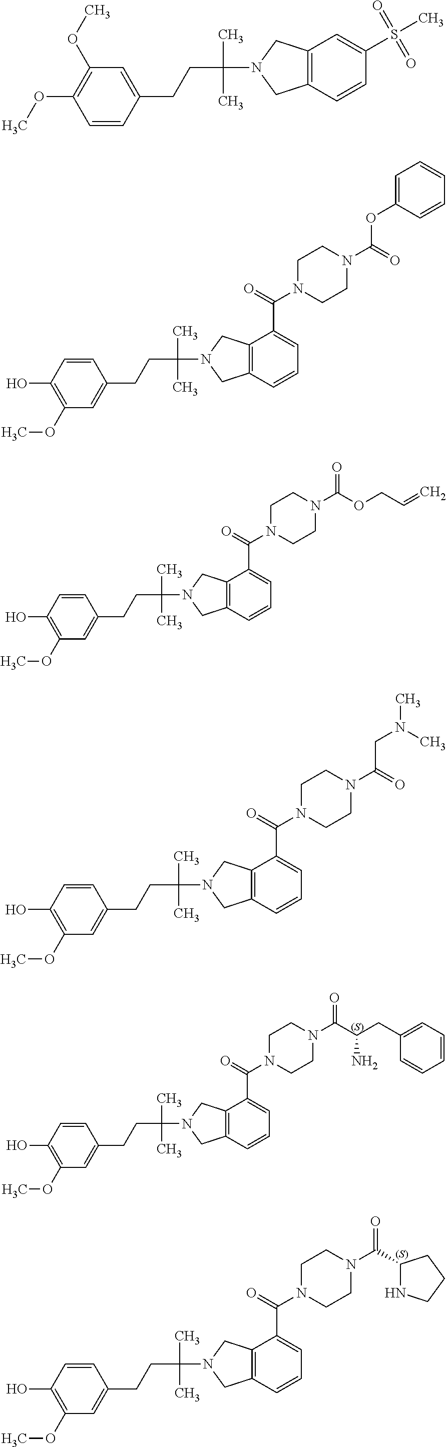

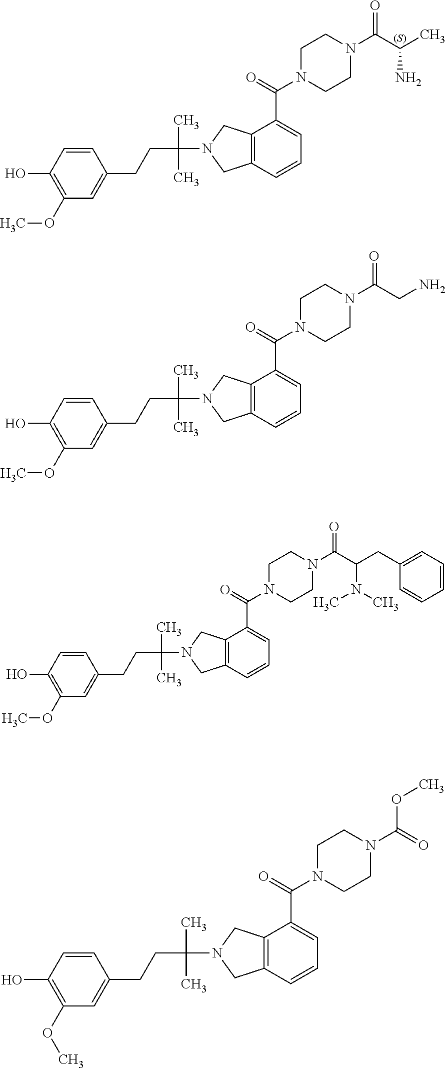

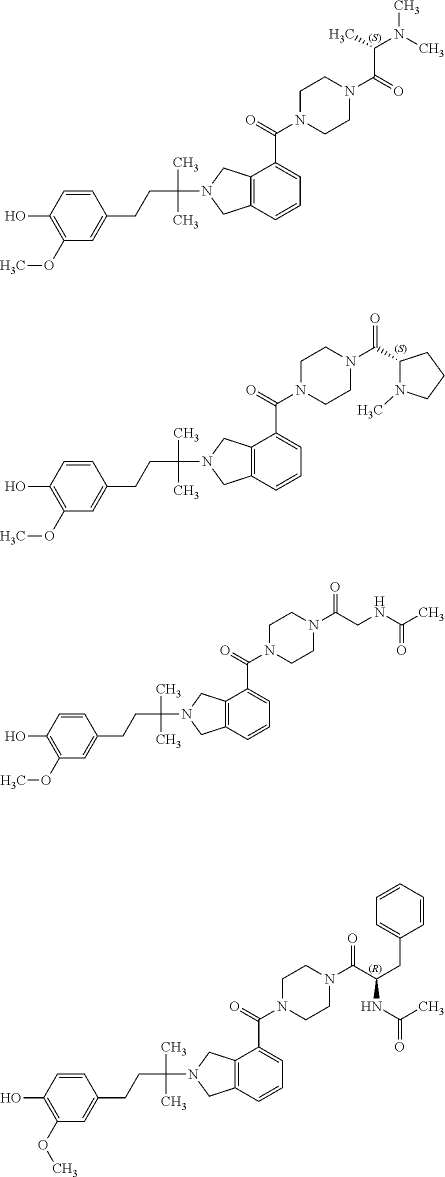

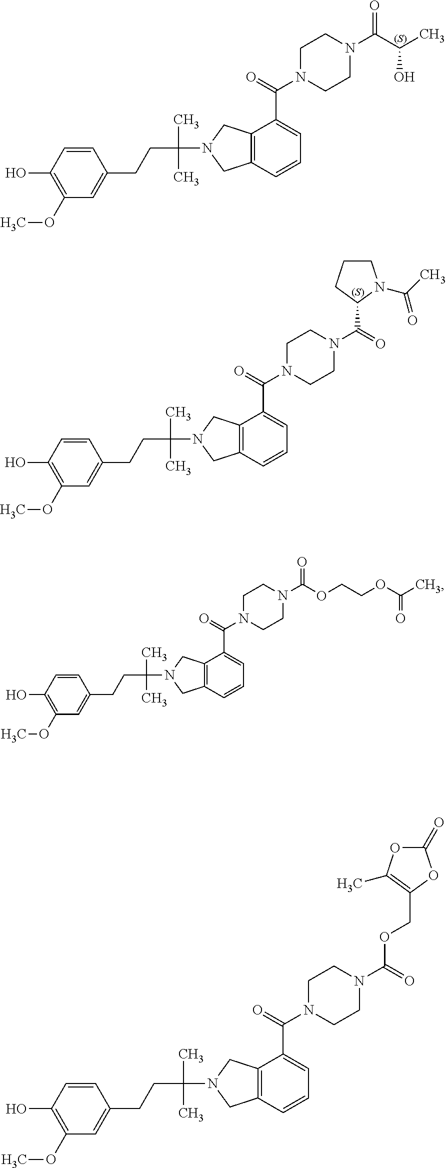

In some embodiments a compound for use in the invention is a compound selected from the group consisting of

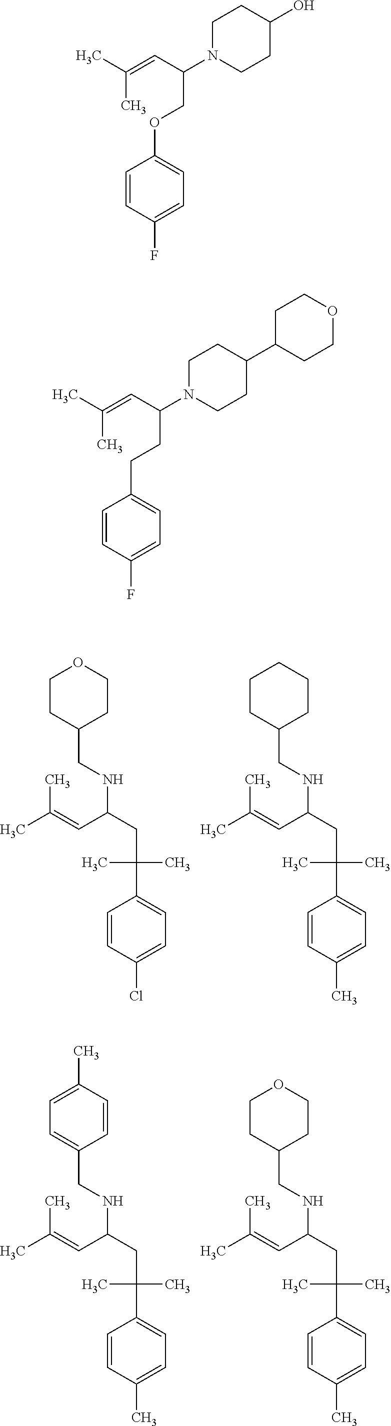 [SMILES: CC(C)=CC(COc1ccc(F)cc1)N1CCC(O)CC1 ; CC(C)=CC(CCc1ccc(F)cc1)N1CCC(C2CCOCC2)CC1 ; CC(C)=CC(CC(C)(C)c1ccc(Cl)cc1)NCC1CCOCC1 ; CC(C)=CC(CC(C)(C)c1ccc(C)cc1)NCC1CCCCC1 ; CC(C)=CC(CC(C)(C)c1ccc(C)cc1)NCc1ccc(C)cc1 ; CC(C)=CC(CC(C)(C)c1ccc(C)cc1)NCC1CCOCC1]

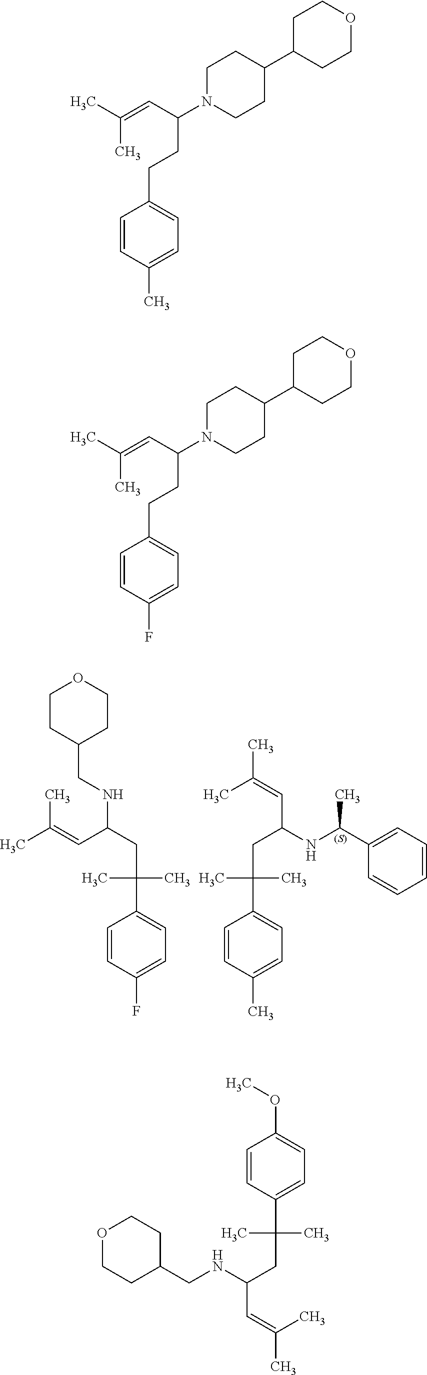

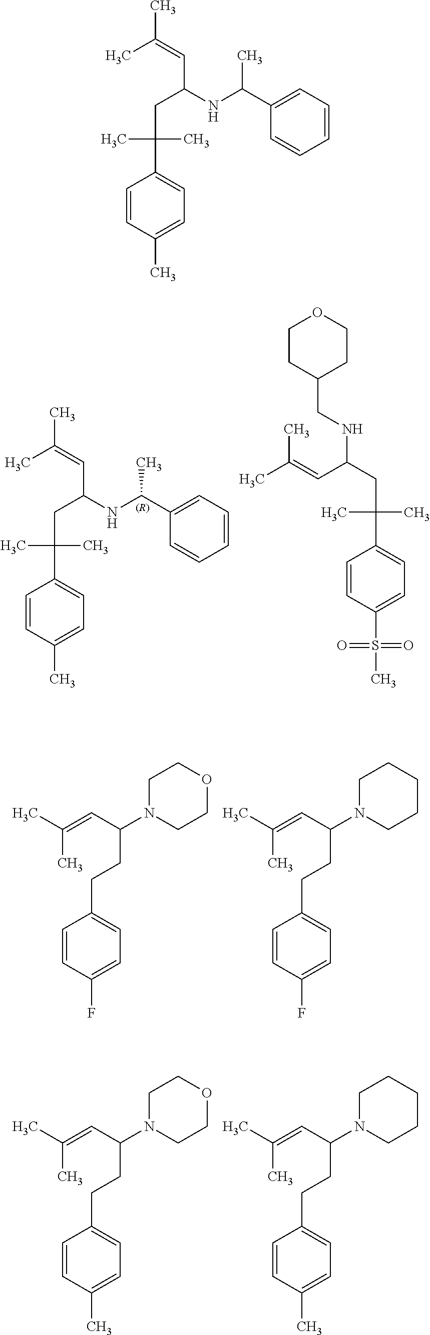

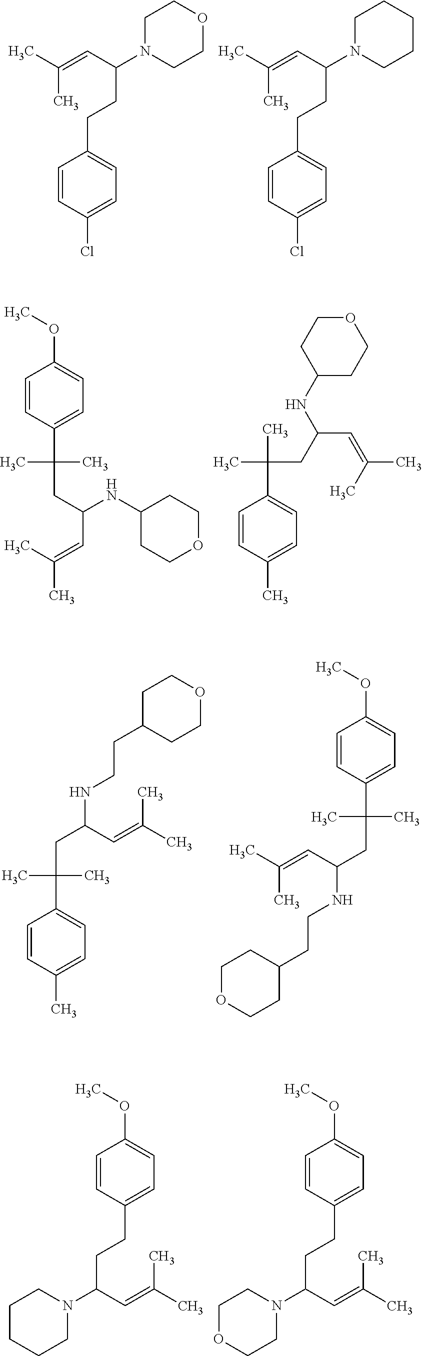

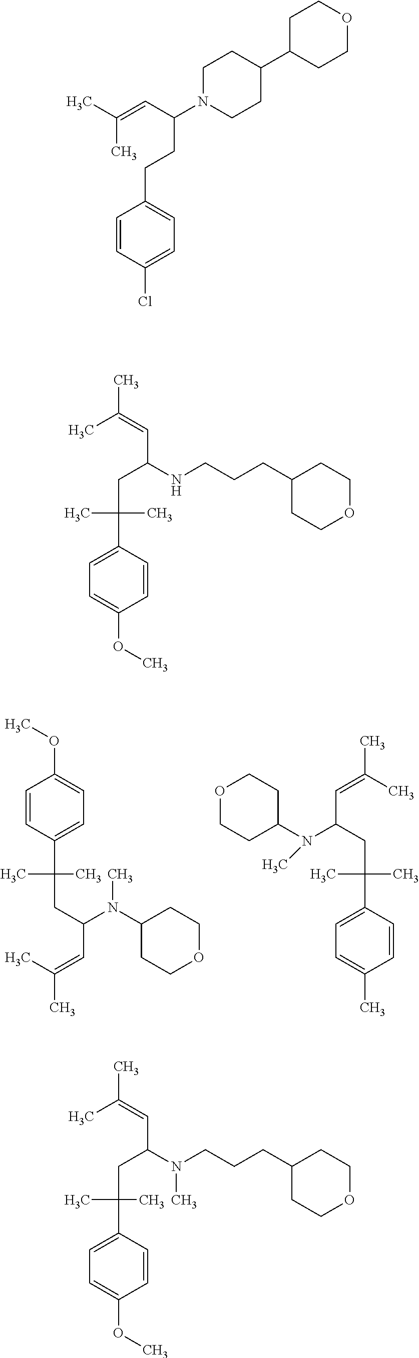 [SMILES: CC(C)=CC(CCc1ccc(Cl)cc1)N1CCC(C2CCOCC2)CC1 ; COc1ccc(C(C)(C)CC(C=C(C)C)NCCCC2CCOCC2)cc1 ; COc1ccc(C(C)(C)CC(C=C(C)C)N(C)C2CCOCC2)cc1 ; CC(C)=CC(CC(C)(C)c1ccc(C)cc1)N(C)C1CCOCC1 ; COc1ccc(C(C)(C)CC(C=C(C)C)N(C)CCCC2CCOCC2)cc1]

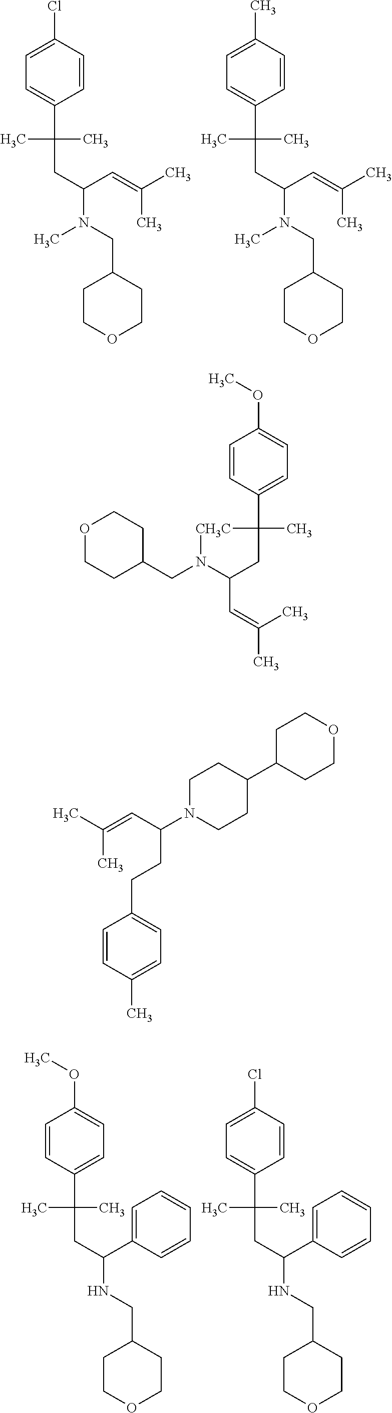

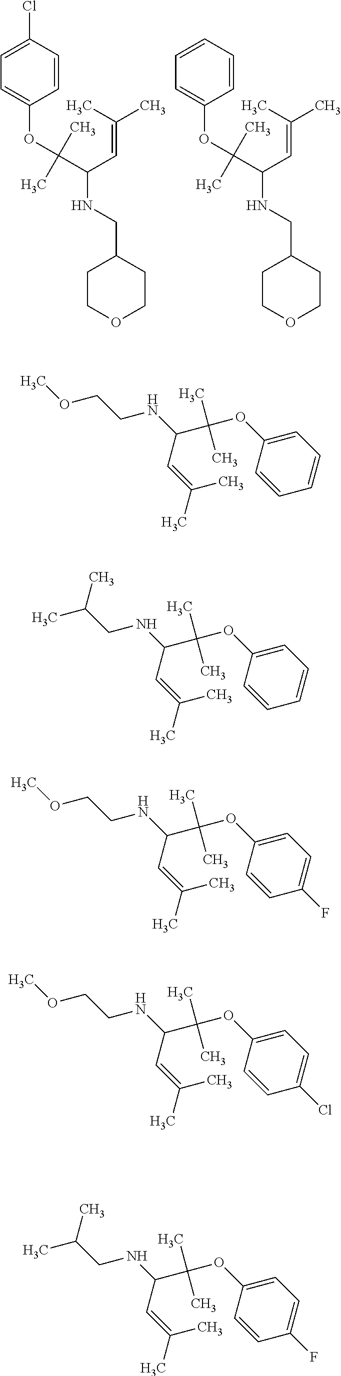

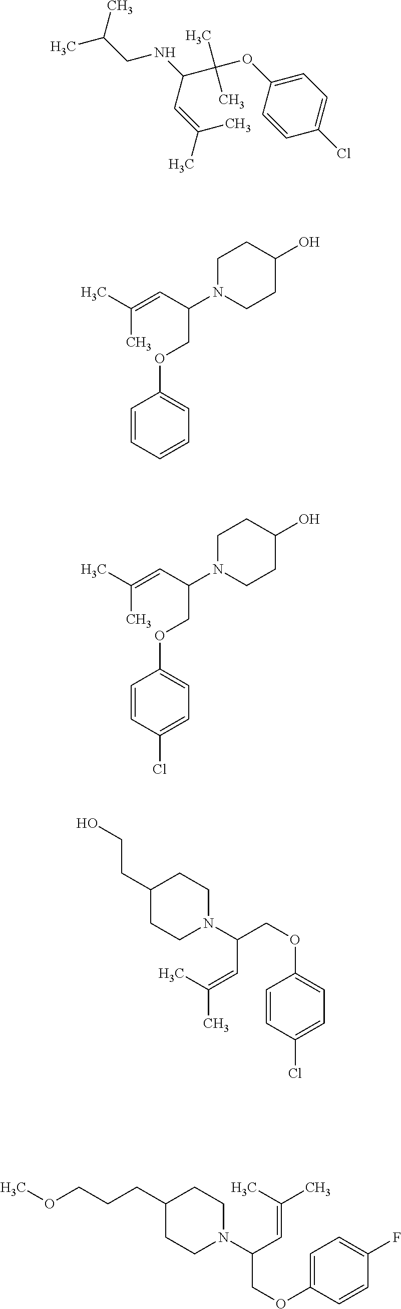 [SMILES: CC(C)=CC(NCC(C)C)C(C)(C)Oc1ccc(Cl)cc1 ; CC(C)=CC(COc1ccccc1)N1CCC(O)CC1 ; CC(C)=CC(COc1ccc(Cl)cc1)N1CCC(O)CC1 ; CC(C)=CC(COc1ccc(Cl)cc1)N1CCC(CCO)CC1 ; COCCCC1CCN(C(C=C(C)C)COc2ccc(F)cc2)CC1]

Additional embodiments include salts, solvates, stereoisomers, prodrugs, and active metabolites of the compounds according to any embodiment described herein.

Some embodiments are directed to free base forms of the compounds according to any embodiment described herein. Other embodiments include salts of such compounds including, for example, pharmaceutically acceptable acid addition salts or pharmaceutically acceptable addition salts of free bases. Examples of pharmaceutically acceptable acid addition salts include, but are not limited to, salts derived from nitric, phosphoric, sulfuric, or hydrobromic, hydrochloric, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Additional salt forms of the compounds described above include salts of amino acids such as arginate and the like and gluconate, galacturonate (see e.g., Berge, et al. “Pharmaceutical Salts,” J. Pharma. Sci. 1977; 66:1).

Pharmaceutically acceptable base addition salts are formed with metals or amines, such as alkali and alkaline earth metals or organic amines. Examples of metals used as cations are sodium, potassium, magnesium, calcium, and the like. Examples of suitable amines include N,N′-dibenzylethylenediamine, chloroprocaine, choline, diethanolamine, dicyclohexylamine, ethylenediamine, N-methylglucamine, and procaine. The base addition salts of said acidic compounds are prepared by contacting the free acid form with a sufficient amount of the desired base to produce the salt in the conventional manner. The free acid form may be regenerated by contacting the salt form with an acid and isolating the free acid.

Various embodiments include total and partial salts, i.e. salts with 1, 2 or 3, preferably 2, equivalents of base per mole of acid of a compound or salt described above, with 1, 2 or 3 equivalents, preferably 1 equivalent, of acid per mole of base of a compound of according to any embodiment described herein. Typically, a pharmaceutically acceptable salt of a compound according to any embodiment described herein, may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound according to any embodiment described herein, and the resulting mixture evaporated to dryness (lyophilized) to obtain the acid addition salt as a solid. Alternatively, a compound according to any embodiment described herein, may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Many organic compounds can form complexes with solvents in which they are reacted or from which they are precipitated or crystallized. These complexes are known as “solvates.” For example, a complex with water is known as a “hydrate.” Various embodiments include solvates of a compound according to any embodiment described herein. In some embodiments, salts of these compounds can form solvates.

Further embodiments include N-oxides of the compounds according to any embodiment described herein. N-oxides include heterocycles containing an otherwise unsubstituted sp² N atom. Examples of such N-oxides include pyridyl N-oxides, pyrimidyl N-oxides, pyrazinyl N-oxides and pyrazolyl N-oxides.

Compounds according to any embodiment described herein, may have one or more chiral centers and, depending on the nature of individual substituents, they can also have geometrical isomers. Thus, embodiments include stereoisomers, diastereomers, and enantiomers of the compounds according to any embodiment described herein. A chiral compound can exist as either an individual enantiomer or as a mixture of enantiomers. A mixture containing equal proportions of the enantiomers is called a “racemic mixture.” A mixture containing unequal portions of the enantiomers is described as having an “enantiomeric excess” (ee) of either the R or S compound. The excess of one enantiomer in a mixture is often described with a % enantiomeric excess. The ratio of enantiomers can also be defined by “optical purity” wherein the degree at which the mixture of enantiomers rotates plane polarized light is compared to the individual optically pure R and S compounds. The compounds can also be a substantially pure (+) or (−) enantiomer of the compounds described herein. In some embodiments, a composition can include a substantially pure enantiomer that is at least 90%, 91%, 92%, 93%, 94%, 95%, 96%, 97%, 98%, or 99% of one enantiomer. In certain embodiments, a composition may include a substantially pure enantiomer that is at least 99.5% one enantiomer.

The description above encompasses all individual isomers of the compounds according to any embodiment described herein, and the description or naming of a particular compound in the specification and claims is intended to include both individual enantiomers and mixtures thereof. Methods for the determination of stereochemistry and the resolution or stereotactic synthesis of stereoisomers are well-known in the art. Diastereomers differ in both physical properties and chemical reactivity. A mixture of diastereomers can be separated into enantiomeric pairs based on solubility, fractional crystallization or chromatographic properties, e.g., thin layer chromatography, column chromatography or HPLC. Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g. solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diastereomeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g. column, at different rates, allowing for their separation

Further embodiments include prodrugs of the compounds according to any embodiment described herein, i.e. compounds which release an active compound according to any of the embodiments described herein, in vivo when administered to a mammalian subject. A prodrug is a pharmacologically active or more typically an inactive compound that is converted into a pharmacologically active agent by a metabolic transformation. Prodrugs of a compound according to any embodiment described herein, are prepared by modifying functional groups present in the compound according to any embodiment described herein, in such a way that the modifications may be cleaved in vivo to release the parent compound. In vivo, a prodrug readily undergoes chemical changes under physiological conditions (e.g. are hydrolyzed or acted on by naturally occurring enzyme(s)) resulting in liberation of the pharmacologically active agent. Prodrugs include compounds according to any embodiment described herein, wherein a hydroxyl, amino, or carboxy group is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds according to any embodiment described herein, or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

In some embodiments, one or more hydrogen atoms of a compound according to any embodiment described herein, is replaced by a deuterium. It is well established that deuteration of physiologically active compounds offer the advantage of retaining the pharmacological profile of their hydrogen counterparts while positively impacting their metabolic outcome. Selective replacement of one or more hydrogen with deuterium, in a compound according to any embodiment described herein, could improve the safety, tolerability and efficacy of the compound when compared to its all hydrogen counterpart.

Methods for incorporation of deuterium into compounds is well established. Using metabolic studies establish in the art, a compound according to any embodiment described herein, can be tested to identify sites for selective placement of a deuterium isotope, wherein the isotope will not be metabolized. Moreover these studies identify sites of metabolism as the location where a deuterium atom would be placed.

### Pharmaceutical Compositions for Use in the Invention

Some embodiments describe a pharmaceutical composition comprising: a compound according to any embodiment described herein, a pharmaceutically acceptable salt thereof, a solvate thereof, a stereoisomer thereof, a prodrug thereof, or an active metabolites thereof; and a pharmaceutically acceptable carrier or diluent. The pharmaceutical compositions can be prepared in a manner well known in the pharmaceutical art, and can be administered by a variety of routes, depending upon whether local or systemic treatment is desired and upon the area to be treated.

While it is possible that a compound as described in any embodiment herein, may be administered as the bulk substance, it is preferable to present the compound in a pharmaceutical formulation, e.g., wherein the active agent is in an admixture with a pharmaceutically acceptable carrier selected with regard to the intended route of administration and standard pharmaceutical practice.

In particular, the disclosure provides a pharmaceutical composition comprising a therapeutically effective amount of at least one compound according to any embodiment described herein, and optionally, a pharmaceutically acceptable carrier.

### Combinations

For the pharmaceutical compositions and methods of the disclosure, a compound according to any embodiment described herein, may be used in combination with other therapies and/or active agents.

In some embodiments, the compound according to any embodiment described herein, can be combined with one or more of an anti-vascular endothelial growth factor (VEGF) treatment, vessel occlusion, and glaucoma treatments. In some embodiments, the compound is combined with a VEGF inhibitor selected from brolucizumab (BEOVU®; Novartis), aflibercept (Eylea®; Regeron), ranibizumab (Lucentis®, Genentech), bevacizumab (Avastin®, Genentech), and pegaptanib (Macugen®, Bausch+Lomb). In some embodiments, the compound is combined with a treatment for vessel occlusion selected from verteporfin (Visudyne®, Bausch+Lomb) and laser treatment. In some embodiments, the compound is combined with a complement cascade therapeutic selected from POT-4 (Compstatin®, Alcon), ARC 1905 (Ophthotech), Eculizumab (Soliris®, Alexion Pharmaceuticals), FCFD4514S (Genentech), TA106 (Taligen Therapeutics and Alexion Pharmaceuticals), JSM-7717 (EvaluatePharma), CR2-tH, and CIINH (ViroPharma). In some embodiments the compound is combined with a treatment for glaucoma selected from brimonidine (Alpagan®; Allergan), apraclonidine (Iopidine®; Novartis), netarsudil (Rhopressa®, Aerie Pharmaceuticals). In some embodiments, the compound is combined with a treatment for glaucoma selected from beta blockers, carbonic anhydrase inhibitors, cholinergics, and prostaglandins.

In some embodiments, the compound according to any embodiment described herein, can be combined with one or more of an anti-vascular endothelial growth factor (VEGF) treatment, vessel occlusion, and glaucoma treatments. In some embodiments, the compound is combined with a VEGF inhibitor selected from brolucizumab (BEOVU®; Novartis), aflibercept (Eylea®; Regeron), ranibizumab (Lucentis®, Genentech), bevacizumab (Avastin®, Genentech), and pegaptanib (Macugen®, Bausch+Lomb). In some embodiments, the compound is combined with a treatment for vessel occlusion selected from verteporfin (Visudyne®, Bausch+Lomb) and laser treatment. In some embodiments, the compound is combined with a complement cascade therapeutic selected from POT-4 (Compstatin®, Alcon), ARC 1905 (Ophthotech), Eculizumab (Soliris®, Alexion Pharmaceuticals), FCFD4514S (Genentech), TA106 (Taligen Therapeutics and Alexion Pharmaceuticals), JSM-7717 (EvaluatePharma), CR2-fi, and CIINH (ViroPharma). In some embodiments the compound is combined with a treatment for glaucoma selected from brimonidine (Alpagan®; Allergan), apraclonidine (Iopidine®; Novartis), netarsudil (Rhopressa®, Aerie Pharmaceuticals). In some embodiments, the compound is combined with a treatment for glaucoma selected from beta blockers, carbonic anhydrase inhibitors, cholinergics, and prostaglandins.

Accordingly, the disclosure provides, in a further aspect, a pharmaceutical composition comprising at least one compound according to any embodiment described herein, or pharmaceutically acceptable derivative thereof; a second active agent; and, optionally a pharmaceutically acceptable carrier.

When combined in the same formulation it will be appreciated that the two or more compounds must be stable and compatible with each other and the other components of the formulation. When formulated separately they may be provided in any convenient formulation, in such manner as are known for such compounds in the art.

Preservatives, stabilizers, dyes and flavoring agents may be provided in any pharmaceutical composition described herein. Examples of preservatives include sodium benzoate, ascorbic acid and esters of p-hydroxybenzoic acid. Antioxidants and suspending agents may be also used.

With respect to combinations including biologics such as monoclonal antibodies or fragments, suitable excipients will be employed to prevent aggregation and stabilize the antibody or fragment in solution with low endotoxin, generally for parenteral administration, for example, intravenous, administration. For example, see Formulation and Delivery Issues for Monoclonal Antibody Therapeutics, Daugherty et al., in Current Trends in Monoclonal Antibody Development and Manufacturing, Part 4, 2010, Springer, New York pp 103-129.

The compounds according to any embodiment described herein, may be milled using known milling procedures such as wet milling to obtain a particle size appropriate for tablet formation and for other formulation types. Finely divided (nanoparticulate) preparations of the compounds may be prepared by processes known in the art, for example see WO 02/00196 (SmithKline Beecham).

Compounds according to any embodiment described herein, or pharmaceutically acceptable salts thereof, a solvate thereof, a stereoisomer thereof, a prodrug thereof, or an active metabolites thereof, can be formulated for any route of administration.

### Routes of Administration and Unit Dosage Forms

The routes for administration (delivery) include, but are not limited to, one or more of: local ocular (e.g. subconjunctival, intravitreal, retrobulbar, intracameral), oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc.

Therefore, the pharmaceutical compositions according to any embodiment described herein, include those in a form especially formulated for the mode of administration. In certain embodiments, the pharmaceutical compositions of the disclosure are formulated in a form that is suitable for oral delivery. In some embodiments, the compound is an orally bioavailable compound, suitable for oral delivery. In other embodiments, the pharmaceutical compositions of the disclosure are formulated in a form that is suitable for parenteral delivery.

The compounds according to any embodiment described herein, may be formulated for administration in any convenient way for use in human or veterinary medicine and the disclosure therefore includes within its scope pharmaceutical compositions comprising a compound according to any embodiment described herein, adapted for use in human or veterinary medicine. Such pharmaceutical compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different pharmaceutical composition/formulation requirements depending on the different delivery systems. It is to be understood that not all of the compounds need to be administered by the same route. Likewise, if the pharmaceutical composition comprises more than one active component, then those components may be administered by different routes. By way of example, the pharmaceutical composition of the disclosure may be formulated to be delivered via a local ocular route, for example, as a subconjunctival ocular injection or intravitreal ocular injection, in which the pharmaceutical composition is formulated for delivery for injection into the eye. Alternatively, the formulation may be designed to be delivered systemically, in which the pharmaceutical composition is formulated for delivery by, for example, an intravenous or oral routes. Alternatively, the formulation may be designed to be delivered by multiple routes.

The combination of a compound according to any embodiment described herein, and an antibody or antibody fragment molecule can be formulated and administered by any of a number of routes and are administered at a concentration that is therapeutically effective in the indication or for the purpose sought. To accomplish this goal, the antibodies may be formulated using a variety of acceptable excipients known in the art. Typically, the antibodies are administered by injection, for example, intravenous injection. Methods to accomplish this administration are known to those of ordinary skill in the art. For example, Gokarn et al., 2008, J Pharm Sci 97(8):3051-3066, incorporated herein by reference, describe various high concentration antibody self buffered formulations. For example, monoclonal antibodies in self buffered formulation at e.g., 50 mg/mL mAb in 5.25% sorbitol, pH 5.0; or 60 mg/mL mAb in 5% sorbitol, 0.01% polysorbate 20, pH 5.2; or conventional buffered formulations, for example, 50 mg/mL mAb1 in 5.25% sorbitol, 25 or 50 mM acetate, glutamate or succinate, at pH 5.0; or 60 mg/mL in 10 mM acetate or glutamate, 5.25% sorbitol, 0.01% polysorbate 20, pH 5.2; other lower concentration formulations can be employed as known in the art.

Because some compounds of the disclosure cross the blood brain barrier they can be administered by a variety of methods including for example systemic (e.g., by iv, SC, oral, mucosal, transdermal route) or localized methods (e.g., intracranially).

Where the compound according to any embodiment described herein, is to be administered directly to the eye, the compound may be administered topically to the eye or eye lid, for example, using drops, an ointment, a cream, a gel, a suspension, etc. The compound(s) may be formulated with excipients such as methylcellulose, hydroxypropyl methylcellulose, hydroxypropyl cellulose, polyvinyl pyrrolidine, neutral poly(meth)acrylate esters, and other viscosity-enhancing agents. The compounds(s) may be injected into the eye, for example, injection under the conjunctiva or tenon capsule, intravitreal injection, or retrobulbar injection. The compounds(s) may be administered with a slow release drug delivery system, such as polymers, matrices, microcapsules, or other delivery systems formulated from, for example, glycolic acid, lactic acid, combinations of glycolic and lactic acid, liposomes, silicone, polyanliydride polyvinyl acetate alone or in combination with polyethylene glycol, etc. The delivery device can be implanted intraocularly, for example, implanted under the conjunctiva, implanted in the wall of the eye, sutured to the sclera, for long-term drug delivery.

Pharmaceutically acceptable excipients and additives for ophthalmic use are known to the person skilled in the art, (carriers, stabilizers, solubilizers, tonicity enhancing agents, buffer substances, preservatives, thickeners, complexing agents and other excipients). Examples of such additives and excipients can be found in U.S. Pat. Nos. 5,891,913, 5,134,124 and 4,906,613. Pharmaceutical compositions of the present invention in some embodiments are prepared, for example by mixing the active agent with the corresponding excipients and/or additives to form corresponding ophthalmic compositions. The compound according to any embodiment described herein can be administered in the form of eye drops, the active agent being conventionally dissolved, for example, in a carrier. The solution is, where appropriate, adjusted and/or buffered to the desired pH and, where appropriate, a stabilizer, a solubilizer or a tonicity enhancing agent is added. Where appropriate, preservatives and/or other excipients are added to an ophthalmic formulation of the invention.

Where the compound according to any embodiment described herein, is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, compounds according to any embodiment described herein, prepared for oral administration may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). In some embodiments, the aqueous enteric coating layer is a methacrylic acid copolymer.

Where appropriate, the pharmaceutical compositions according to any embodiment described herein, can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the pharmaceutical compositions according to any embodiment described herein, may be administered in the form of tablets or lozenges, which can be formulated in a conventional manner.

Where the pharmaceutical composition according to any embodiment described herein, is to be administered parenterally, such administration includes without limitation: intravenously, intraarterially, intrathecally, intraventricularly, intracranially, intramuscularly or subcutaneously administering the compound of the disclosure; and/or by using infusion techniques. Antibodies or fragments are typically administered parenterally, for example, intravenously.

Pharmaceutical compositions according to any embodiment described herein, suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound according to any embodiment described herein, is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound according to any embodiment described herein, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the compounds and desired excipients for subsequent preparation of sterile solutions.

The compounds according to any embodiment described herein, may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The pharmaceutical compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds according to any embodiment described herein, can be administered in the form of tablets, capsules, troches, ovules, elixirs, solutions or suspensions, for immediate-, delayed-, modified-, sustained-, pulsed- or controlled-release applications.

The compounds according to any embodiment described herein, may also be presented for human or veterinary use in a form suitable for oral or buccal administration, for example in the form of solutions, gels, syrups, or suspensions, or a dry powder for reconstitution with water or other suitable vehicle before use. Solid pharmaceutical compositions such as tablets, capsules, lozenges, troches, pastilles, pills, boluses, powder, pastes, granules, bullets or premix preparations may also be used. Solid and liquid pharmaceutical compositions for oral use may be prepared according to methods well-known in the art. Such pharmaceutical compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The pharmaceutical compositions according to any embodiment described herein, may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the pharmaceutical compositions, e.g., act as both binders and disintegrants.

Examples of pharmaceutically acceptable disintegrants for oral pharmaceutical compositions according to any embodiment described herein, include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral pharmaceutical compositions according to any embodiment described herein, include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium aluminum silicate, polyethylene glycol or bentonite.

Examples of pharmaceutically acceptable fillers for oral pharmaceutical compositions according to any embodiment described herein, include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the pharmaceutical compositions according to any embodiment described herein, include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral pharmaceutical compositions according to any embodiment described herein, include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral pharmaceutical compositions according to any embodiment described herein, include, but are not limited to, synthetic and natural dyes such as titanium dioxide, beta-carotene and extracts of grapefruit peel.

Examples of useful pharmaceutically acceptable coatings for the oral pharmaceutical compositions according to any embodiment described herein, typically used to facilitate swallowing, modify the release properties, improve the appearance, and/or mask the taste of the pharmaceutical compositions include, but are not limited to, hydroxypropylmethylcellulose, hydroxypropylcellulose and acrylate-methacrylate copolymers.

Suitable examples of pharmaceutically acceptable sweeteners for the oral pharmaceutical compositions according to any embodiment described herein, include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, a compounds according to any embodiment described herein, can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of a compound according to any embodiment described herein, and a suitable powder base such as lactose or starch.

For topical administration by inhalation a compounds according to any embodiment described herein, may be delivered for use in human or veterinary medicine via a nebulizer.

The pharmaceutical compositions of the disclosure may contain from 0.01 to 99% weight per volume of the active material. For topical administration, for example, the pharmaceutical composition will generally contain from 0.01-10%, more preferably 0.01-1% of the active material.

A compound according to any embodiment described herein, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form, according to any embodiment described herein, may be administered according to a dosage and administration regimen defined by routine testing in the light of the guidelines given above in order to obtain optimal activity while minimizing toxicity or side effects for a particular patient. The dosage of the compounds or unit dosage form may vary according to a variety of factors such as underlying disease conditions, the individual's condition, weight, sex and age, and the mode of administration. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter is within the scope of the disclosure. Clinically significant attenuation or amelioration means perceptible to the patient and/or to the physician.

In some embodiments, the amount of the compound to be administered can range between about 0.01 and about 25 mg/kg/day. Generally, dosage levels of between 0.01 to 25 mg/kg of body weight daily are administered to the patient, e.g., humans. In some embodiments the therapeutically effective amount is between a lower limit of about 0.01 mg/kg of body weight, about 0.1 mg/kg of body weight, about 0.2 mg/kg of body weight, about 0.3 mg/kg of body weight, about 0.4 mg/kg of body weight, about 0.5 mg/kg of body weight, about 0.60 mg/kg of body weight, about 0.70 mg/kg of body weight, about 0.80 mg/kg of body weight, about 0.90 mg/kg of body weight, about 1 mg/kg of body weight, about 2.5 mg/kg of body weight, about 5 mg/kg of body weight, about 7.5 mg/kg of body weight, about 10 mg/kg of body weight, about 12.5 mg/kg of body weight, about 15 mg/kg of body weight, about 17.5 mg/kg of body weight, about 20 mg/kg of body weight, about 22.5 mg/kg of body weight, and about 25 mg/kg of body weight; and an upper limit of 25 mg/kg of body weight, about 22.5 mg/kg of body weight, about 20 mg/kg of body weight, about 17.5 mg/kg of body weight, about 15 mg/kg of body weight, about 12.5 mg/kg of body weight, about 10 mg/kg of body weight, about 7.5 mg/kg of body weight, about 5 mg/kg of body weight, about 2.5 mg/kg of body weight, about 1 mg/kg of body weight, about 0.9 mg/kg of body weight, about 0.8 mg/kg of body weight, about 0.7 mg/kg of body weight, about 0.6 mg/kg of body weight, about 0.5 mg/kg of body weight, about 0.4 mg/kg of body weight, about 0.3 mg/kg of body weight, about 0.2 mg/kg of body weight, about 0.1 mg/kg of body weight, and about 0.01 mg/kg of body weight. In some embodiments, the therapeutically effective amount is about 0.1 mg/kg/day to about 10 mg/kg/day; in some embodiments the therapeutically effective amount is about 0.2 and about 5 mg/kg/day. It will be understood that the pharmaceutical formulations of the disclosure need not necessarily contain the entire amount of the compound that is effective in treating the disorder, as such effective amounts can be reached by administration of a plurality of divided doses of such pharmaceutical formulations. The compounds may be administered on a regimen of 1 to 4 times per day, such as once, twice, three times or four times per day.

In some embodiments of the disclosure, a compound according to any embodiment described herein, is formulated in capsules or tablets, usually containing about 10 to about 200 mg of the compounds. In some embodiments the capsule or tablet contains between a lower limit of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg; about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, and about 200 mg, and an upper limit of about 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 95 mg, about 90 mg; about 85 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, about 15 mg, and about 10 mg of a compound according to any embodiment herein.

In some embodiments, a compound according to any embodiment herein is administered to a patient at a total daily dose of 50 mg to 500 mg. In some embodiments, the daily dose is between a lower limit of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg; about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg; about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, 300 mg, about 305 mg, about 310 mg, about 315 mg; about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395, about 400 mg, about 405 mg, about 410 mg, about 415 mg; about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, and about 500 mg and an upper limit of about 500 mg, about 495 mg, about 490 mg, about 485 mg, about 480 mg, about 475 mg, about 470 mg, about 465 mg, about 460 mg, about 455 mg, about 450 mg, about 445 mg, about 440 mg, about 435 mg, about 430 mg, about 425 mg, about 420 mg, about 415 mg, about 410 mg, about 405 mg, about 400 mg, about 395 mg, about 390 mg, about 385 mg, about 380 mg, about 375 mg, about 370 mg, about 365 mg, about 360 mg, about 355 mg, about 350 mg, about 345 mg, about 340 mg, about 335 mg, about 330 mg, about 325 mg, about 320 mg, about 315 mg, about 310 mg, about 305 mg about 300 mg, about 295 mg, about 290 mg, about 285 mg, about 280 mg, about 275 mg, about 270 mg, about 265 mg, about 260 mg, about 255 mg, about 250 mg, about 245 mg, about 240 mg, about 235 mg, about 230 mg, about 225 mg, about 220 mg, about 215 mg, about 210 mg, about 205 mg 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 95 mg, about 90 mg; about 85 mg, about 80 mg, about 75 mg, about 70 mg, about 65 mg, about 60 mg, about 55 mg, and about 50 mg of a compound according to any embodiment herein. In some embodiments, the total daily dose is about 50 mg to 150 mg. In some embodiments, the total daily dose is about 50 mg to 250 mg. In some embodiments, the total daily dose is about 50 mg to 350 mg. In some embodiments, the total daily dose is about 50 mg to 450 mg. In some embodiments, the total daily dose is about 50 mg.

A pharmaceutical composition for parenteral administration contains from about 0.01% to about 100% by weight of the active compound according to any embodiment described herein, based upon 100% weight of total pharmaceutical composition.

Generally, transdermal dosage forms contain from about 0.01% to about 100% by weight of the active compound according to any embodiment described herein, versus 100% total weight of the dosage form.

The pharmaceutical composition or unit dosage form may be administered in a single daily dose, or the total daily dosage may be administered in divided doses. In addition, co administration or sequential administration of another compound for the treatment of the disorder may be desirable. To this purpose, the combined active principles are formulated into a simple dosage unit.

In some embodiments, a compound according to any embodiment described herein, generally inhibits the Abeta effect on neurons. In some embodiments, the compounds describe above have an IC₅₀ for inhibition of Abeta effect of less than about 100 μM, about 50 μM, about 20 μM, about 15 μM, about 10 μM, about 5 μM, about 1 μM, about 500 nM, about 100 nM, about 50 nM, or about 10 nM on neurons (such as neurons in the brain), amyloid assembly or disruption thereof, and amyloid (including amyloid oligomer) binding, and amyloid deposition. In some embodiments, a compound according to any embodiment described herein, may have an IC₅₀ for inhibition of the activity/effect of Abeta species such as oligomers of less than about 100 μM, about 50 μM, about 20 μM, about 15 μM, about 10 μM, about 5 μM, about 1 μM, about 500 nM, about 100 nM, about 50 nM, or about 10 nM on neurons (such as central nervous system neurons).

A compound according to any embodiment described herein, may inhibit the Abeta effect by specifically binding to a sigma-2 receptor. A compound can be said to be “specific” for a sigma-2 receptor when it binds with a binding affinity that is at least 10% greater than to the sigma-1 receptor, even though the compound is capable of binding both sigma-1 and sigma-2 receptor. The compounds of such embodiments may exhibit a specificity of at least 10%, 20%, 30%, 40%, 50%, 60%, 70%, 80%, 90%, 100%, 200%, 300%, 400%, 500%, or 1000% greater for sigma-2 receptor than sigma-1 receptor.

In some embodiments, percentage inhibition by a compound according to any embodiment described herein, of one or more of the effects of Abeta species such as oligomers on RPEs and RGCs, such as amyloidinduced oxidative stress, cell damage, cell death, and abnormalities in membrane trafficking mediated by Abeta oligomer can be about 1% to about 20%, about 20% to about 50%, about 1% to about 50%, or about 1% to about 80% as measured at a concentration of from 10 nM to 10 μM. Inhibition can be assessed for example by quantifying defects in photoreceptor outer segment (POS) trafficking prior to and after exposure to an amyloid beta species or quantifying the defects in photoreceptor outer segment (POS) trafficking in the presence of both of the compound according to any embodiment described herein, and the Abeta species wherein the compound according to any embodiment described herein, is simultaneous with, or precedes or follows, Abeta species exposure. As another example, inhibition can be assessed by determining membrane trafficking and comparing one or more parameters that measure rate and extent of cell senescence, or other indicators of cell health and metabolism in the presence and absence of an Abeta species and in the presence and absence of a compound according to any embodiment described herein.

In some embodiments, an assay is used to determine if a compound according to any embodiment described herein, can bind to a sigma-2 receptor. In some embodiments, the method further comprises determining whether the compound that binds to a sigma-2 receptor acts as a functional antagonist at a sigma-2 receptor by inhibiting soluble Aβ oligomer induced cytotoxicity.

Any form of amyloid β may be used in the practice of the screening methods and of the assays according to the disclosure, including amyloid β monomers, oligomers, fibrils, as well as amyloid β associated with proteins (“protein complexes”) and more generally amyloid β assemblies. For example, screening methods can employ various forms of soluble amyloid β oligomers as disclosed, for example, in U.S. patent application Ser. No. 13/021,872; U.S. Patent Publication 2010/0240868; International Patent Application WO/2004/067561; International Patent Application WO/2010/011947; U.S. Patent Publication 20070098721; U.S. Patent Publication 20100209346; International Patent Application WO/2007/005359; U.S. Patent Publication 20080044356; U.S. Patent Publication 20070218491; WO/2007/126473; U.S. Patent Publication 20050074763; International Patent Application WO/2007/126473, International Patent Application WO/2009/048631, and U.S. Patent Publication 20080044406, U.S. Pat. Nos. 7,902,328 and 6,218,506, each of which is incorporated herein by reference.

Amyloid β forms, including monomers or oligomers of amyloid β may be obtained from any source. For example, in some embodiments, commercially available amyloid β monomers and/or amyloid β oligomers may be used in the aqueous solution, and in other embodiments, amyloid β monomers and/or amyloid β oligomers that are used in the aqueous protein solution can be isolated and purified by the skilled artisan using any number of known techniques. In general, the amyloid β monomers and/or amyloid β oligomers used in the preparation of the aqueous solution of proteins and amyloid β of various embodiments may be soluble in the aqueous solution. Therefore, both the proteins of the aqueous solution and the amyloid β may be soluble.

The amyloid β added may be of any isoform. For example, in some embodiments, the amyloid β monomers may be amyloid β 1-42, and in other embodiments the amyloid β monomers may be amyloid β 1-40. In still other embodiments, the amyloid β may be amyloid β 1-39 or amyloid β 1-41. Hence, the amyloid β of various embodiments may encompass any C-terminal isoform of amyloid β. Yet other embodiments include amyloid β in which the N-terminus has been frayed, and in some embodiments, the N-terminus of any of amyloid β C-terminal isomers described above may be amino acid 2, 3, 4, 5, or 6. For example, amyloid β 1-42 may encompass amyloid β 2-42, amyloid β 3-42, amyloid β 4-42, or amyloid β 5-42 and mixtures thereof, and similarly, amyloid β 1-40 may encompass amyloid β 2-40, amyloid β 3-40, amyloid β 4-40, or amyloid β 5-40.

The amyloid β forms used in various embodiments may be wild type, i.e. having an amino acid sequence that is identical to the amino acid sequence of amyloid β synthesized in vivo by the majority of the population, or in some embodiments, the amyloid β may be a mutant amyloid β. Embodiments are not limited to any particular variety of mutant amyloid β. For example, in some embodiments, the amyloid β introduced into the aqueous solution may include a known mutation, such as, for example, amyloid β having the “Dutch” (E22Q) mutation or the “Arctic” (E22G) mutation. Such mutated monomers may include naturally occurring mutations such as, for example, forms of amyloid β isolated from populations of individuals that are predisposed to, for example, Alzheimer's disease, familial forms of amyloid β. In other embodiments, mutant amyloid β monomers may be synthetically produced by using molecular techniques to produce an amyloid β mutant with a specific mutation. In still other embodiments, mutant amyloid β monomers may include previously unidentified mutations such as, for example, those mutants found in randomly generated amyloid β mutants. The term “amyloid β” as used herein, is meant to encompass both wild type forms of amyloid β as well as any of the mutant forms of amyloid β.

In some embodiments, the amyloid β in the aqueous protein solution may be of a single isoform. In other embodiments, various C-terminal isoforms of amyloid β and/or various N-terminal isoforms of amyloid β may be combined to form amyloid β mixtures that can be provided in the aqueous protein solution. In yet other embodiments, the amyloid β may be derived from amyloid precursor protein (APP) that is added to the protein containing aqueous solution and is cleaved in situ, and such embodiments, various isoforms of amyloid β may be contained within the solution. Fraying of the N-terminus and/or removal of C-terminal amino acids may occur within the aqueous solution after amyloid β has been added. Therefore, aqueous solutions prepared as described herein, may include a variety of amyloid β isoforms even when a single isoform is initially added to the solution.

The amyloid β monomers added to the aqueous solution may be isolated from a natural source such as living tissue, and in other embodiments, the amyloid β may be derived from a synthetic source such as transgenic mice or cultured cells. In some embodiments, the amyloid β forms, including monomers, oligomers, or combinations thereof are isolated from normal subjects and/or patients that have been diagnosed with cognitive decline or diseases associated therewith, such as, but not limited to, Alzheimer's disease. In some embodiments, the amyloid β monomers, oligomers, or combinations thereof are Abeta assemblies that have been isolated from normal subjects or diseased patients. In some embodiments, the Abeta assemblies are high molecular weight, e.g. greater than 100 KDa. In some embodiments, the Abeta assemblies are intermediate molecular weight, e.g. 10 to 100 KDa. In some embodiments, the Abeta assemblies are less than 10 kDa.

The amyloid β oligomers of some embodiments may be composed of any number of amyloid β monomers consistent with the commonly used definition of “oligomer.” For example, in some embodiments, amyloid β oligomers may include from about 2 to about 300, about 2 to about 250, about 2 to about 200 amyloid β monomers, and in other embodiments, amyloid β oligomers may be composed from about 2 to about 150, about 2 to about 100, about 2 to about 50, or about 2 to about 25, amyloid β monomers. In some embodiments, the amyloid β oligomers may include 2 or more monomers. The amyloid β oligomers of various embodiments may be distinguished from amyloid β fibrils and amyloid β protofibrils based on the confirmation of the monomers. In particular, the amyloid β monomers of amyloid β oligomers are generally globular consisting of β-pleated sheets whereas secondary structure of the amyloid β monomers of fibrils and protofibrils is parallel β-sheets.

Provided herein is embodiment A, a method of treating dry age-related macular degeneration (dry AMD), comprising administering to a subject in need thereof, a therapeutically effective amount of a compound selected from the group consisting of a compound of Formula I,

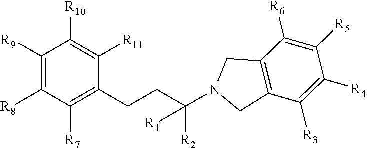

or a pharmaceutically-acceptable salt thereof: wherein: each of R₁ and R₂ is independently selected from H, C₁-C₆ alkyl, or CH₂OR′; wherein each R′ if present in R₁, and R₂ is independently H or C₁-C₆ alkyl; each of R₃, R₄, R₅, and R₆ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁-C₆ alkoxy C₁-C₆alkyl, aryl, heteroaryl, C₃-C₇ cycloalkyl, heterocycloalkyl, alkylaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₃, R₄, R₅, and R₆ is independently selected from the group consisting of H, CH₃, CH₂CH₃, C₃-C₆ alkyl, C₁-C₆ haloalkyl, or optionally substituted aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂, wherein the optionally substituted group is selected from C₁-C₆ alkyl or C₂-C₇ acyl; or R₃ and R₄, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₃ and R₄ are linked together to form a —O—C₁-C₂ methylene-O— group; or R₄ and R₅, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₄ and R₅ are linked together to form a —O—C₁₋₂ methylene-O— group; each of R₇, R₈, R₉, R₁₀, and R₁₁ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁-C₆ alkoxy C₁-C₆ alkyl, aryl, heteroaryl, C₃-C₇ cycloalkyl, heterocycloalkyl, alkylaryl, heteroaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₇, R₈, R₉, R₁₀, and R₁₁ is independently selected from the group consisting of H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂; or R₇ and R₈, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₇ and R₈ are linked together to form a —O—C₁₋₂ methylene-O— group; or R₈ and R₉, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁. C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₈ and R₉ are linked together to form a —O—C₁₋₂ methylene-O— group; each n is independently 0, 1, or 2; with the proviso that R₇, R₈, R₉, R₁₀, and R₁₁ are not all H; and with the proviso that the following compounds, or pharmaceutically acceptable salts thereof are excluded:

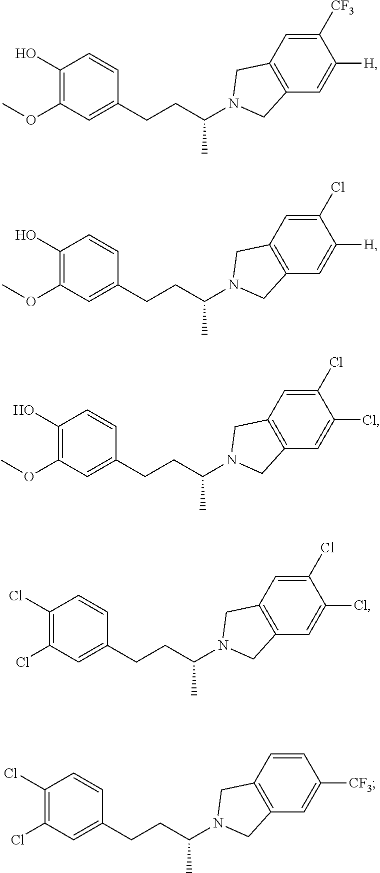

or

Provided herein is embodiment B, a method of treating dry age-related macular degeneration (dry AMD), comprising administering to a subject in need thereof, a therapeutically effective amount of a compound selected from the group consisting of a compound of Formula IA:

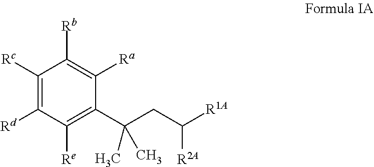

or a pharmaceutically acceptable salt thereof: wherein: each of R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino; R^{IA} is selected from the group consisting of hydrogen, alkyl, phenyl, or —CH═C(CH₃)₂; and R^{2A} is an optionally substituted cyclic amino group.

In an embodiment C, the method of embodiment A, wherein the compound is a compound of Formula I or a pharmaceutically-acceptable salt thereof.

In an embodiment D, the method of any one of embodiments A to C, wherein the compound is

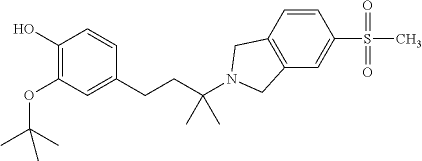 [SMILES: CC(C)(C)Oc1cc(CCC(C)(C)N2Cc3ccc(S(C)(=O)=O)cc3C2)ccc1O]

or a pharmaceutically acceptable salt thereof.

In an embodiment E, the method of any one of embodiments A to D, wherein the pharmaceutically acceptable salt is selected from the group consisting of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

In an embodiment F, the method of any one of the embodiments A to E, wherein the pharmaceutically acceptable salt is the fumarate salt.

In an embodiment G, the method of any one of the embodiments A to F, wherein the compound is

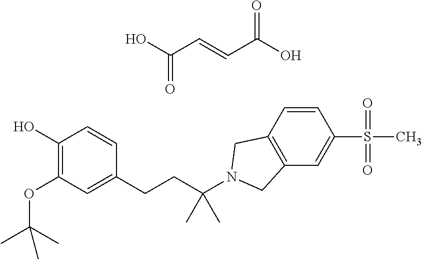 [SMILES: O=C(O)/C=C/C(=O)O ; CC(C)(C)Oc1cc(CCC(C)(C)N2Cc3ccc(S(C)(=O)=O)cc3C2)ccc1O]

In an embodiment H, the method of embodiment B, wherein the compound is a compound of Formula IA or a pharmaceutically-acceptable salt thereof.

In an embodiment I, the method of either of embodiments A or H, wherein the the R^{2A} is optionally substituted piperidinyl.

In an embodiment J, the method of any one of the embodiments A, H, or I, wherein wherein the R^{2A} s selected from the group consisting of

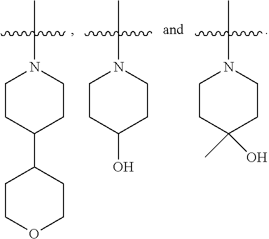

In an embodiment K, the method of any one of embodiments A, H, I, or J, wherein the compound is selected from the group consisting of

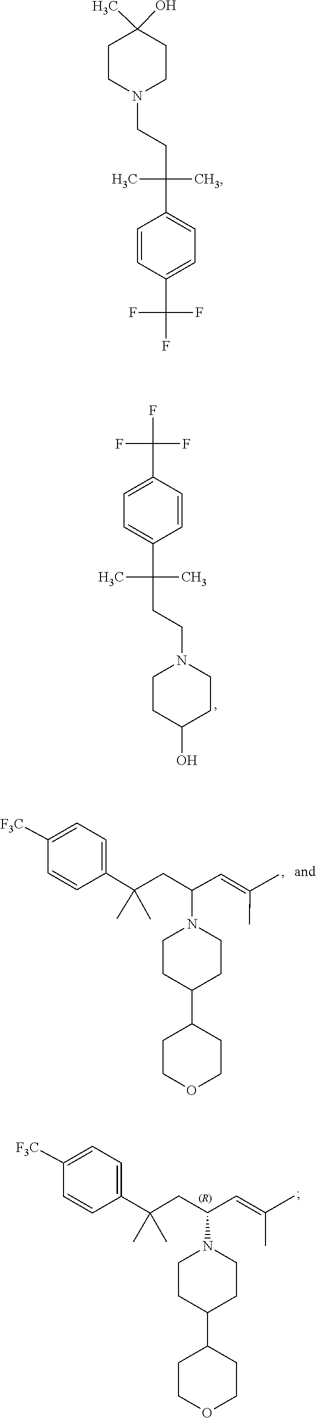

or a pharmaceutically acceptable salt thereof.

Provided herein is embodiment L, a method of treating dry age-related macular degeneration dry (AMD), comprising administering to a subject in need thereof, a therapeutically effective amount of a compound the compound is selected from the group consisting of:

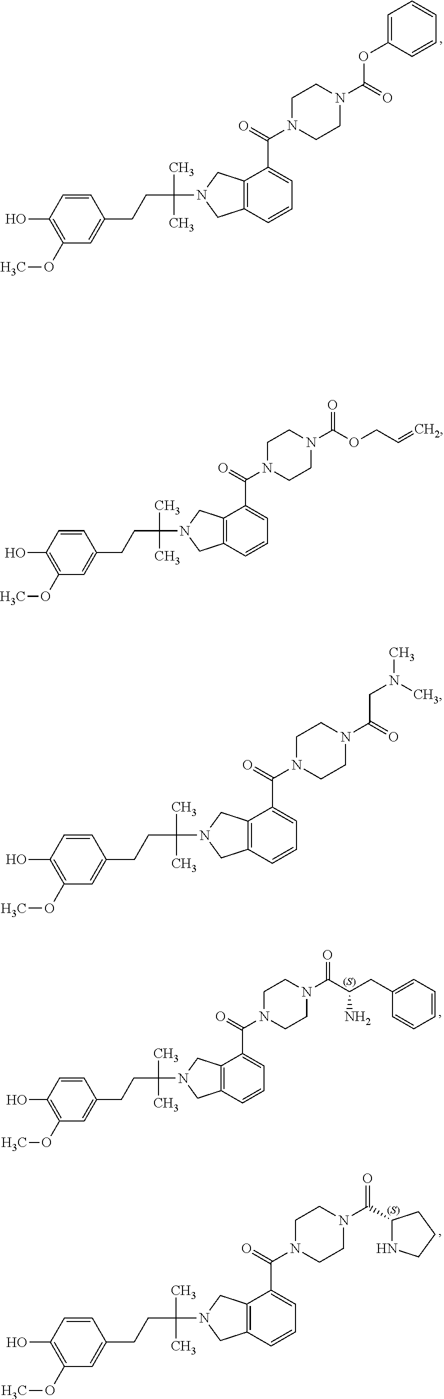

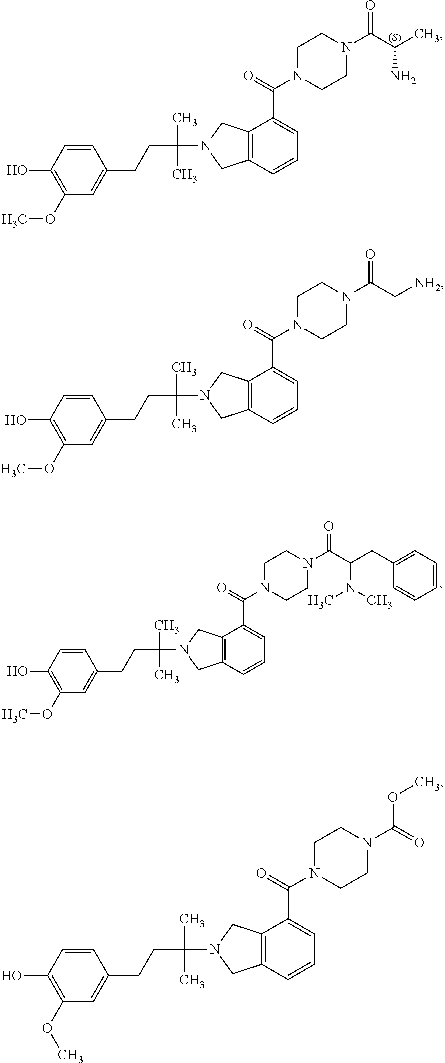

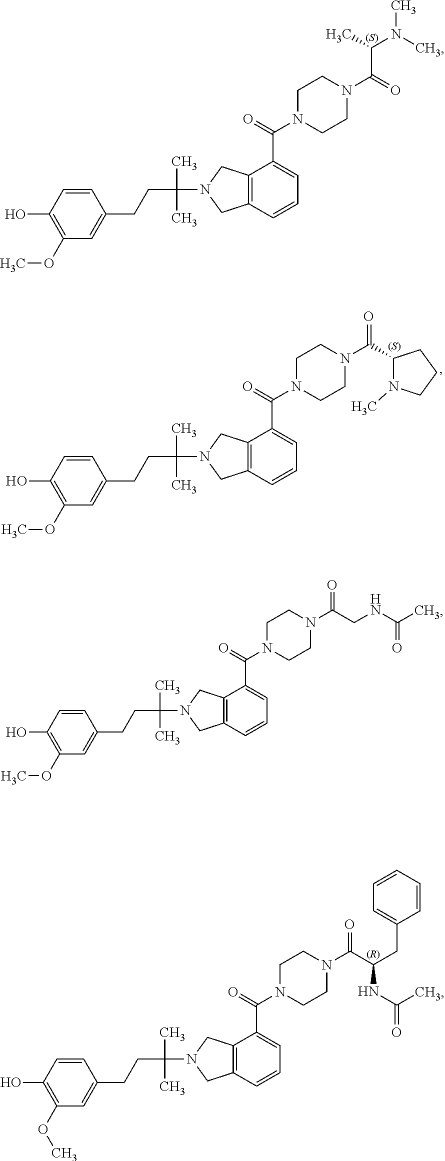

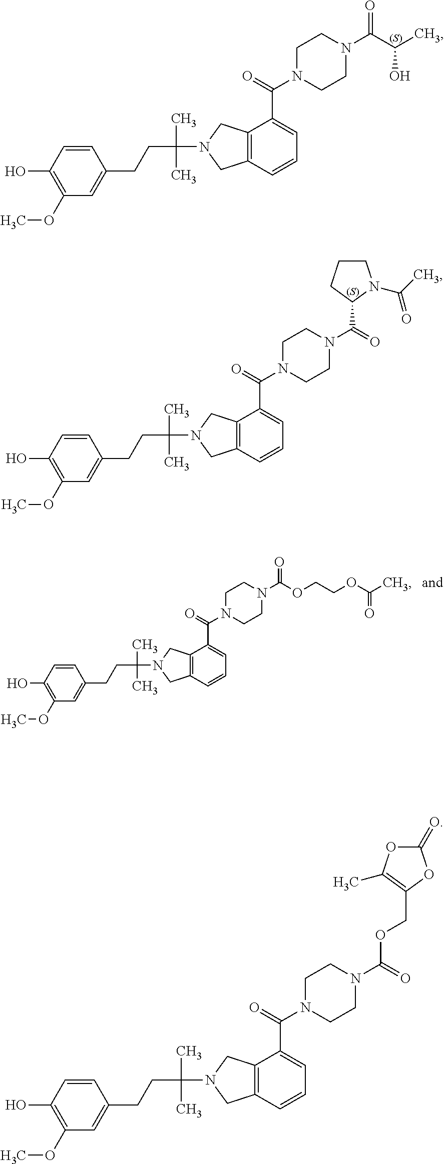

In an embodiment M, a method of treating dry age-related macular degeneration comprising administering to a subject in need thereof, a therapeutically effective amount of a pharmaceutical composition comprising a compound according to any one of embodiments A-L and a pharmaceutically acceptable excipient.

In an embodiment N, a method of treating dry age-related macular degeneration comprising administering to a subject in need thereof, a therapeutically effective amount of a pharmaceutical composition comprising a compound selected from the group comprising:

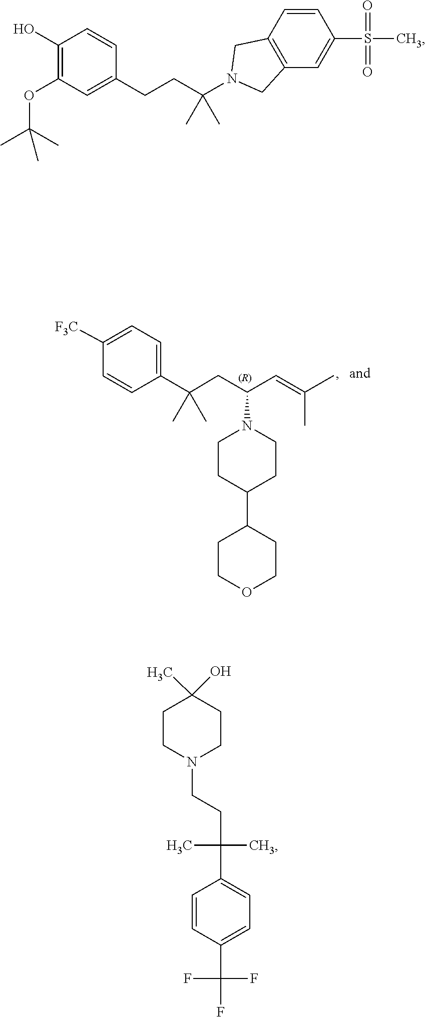

or a pharmaceutically acceptable salt thereof, and a pharmaceutically acceptable excipient.

In an embodiment O, a method of embodiment N, wherein the pharmaceutically acceptable salt is selected from the group consisting of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

In an embodiment P, a method of embodiment N, wherein the pharmaceutically acceptable salt is the fumarate salt.

In an embodiment Q, a method of embodiment P, wherein the compound is

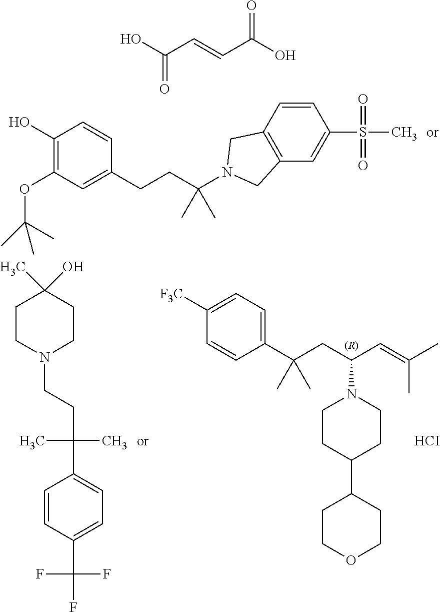

Provided herein is an embodiment R, a use of a compound selected from

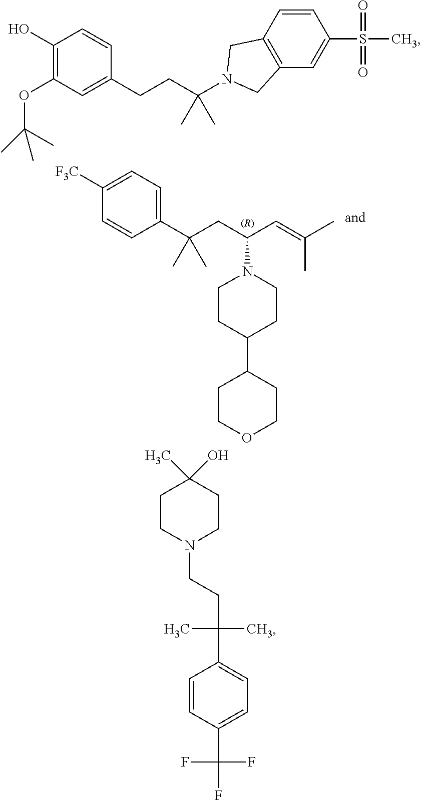

or a pharmaceutically acceptable salt thereof, in the manufacture of a medicament for the treatment of dry age-related macular degeneration.

Provided herein is an embodiment S, a use of a composition comprising a compound selected from

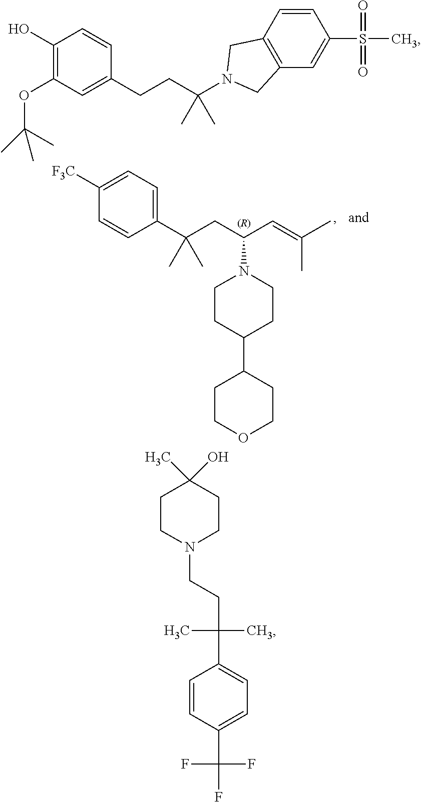

or a pharmaceutically acceptable salt thereof, and a pharmaceutically acceptable excipient; in the manufacture of a medicament for the treatment of dry age-related macular degeneration.

In an embodiment T, the use of a compound or composition of either of embodiments R or S, wherein the compound is a pharmaceutically-acceptable salt thereof.

In an embodiment U, the use of any one of the embodiments R to T, wherein the pharmaceutically acceptable salt is selected from the group consisting of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

In an embodiment V, the use of embodiment U, wherein the pharmaceutically acceptable salt is the fumarate salt.

In an embodiment W, the use of either of embodiments R or S, wherein the compound

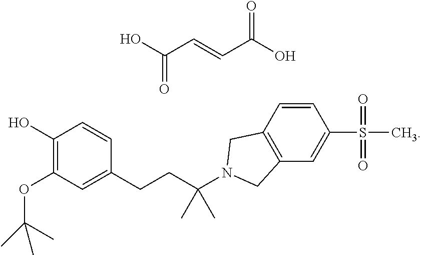 [SMILES: O=C(O)/C=C/C(=O)O ; CC(C)(C)Oc1cc(CCC(C)(C)N2Cc3ccc(S(C)(=O)=O)cc3C2)ccc1O]

In an embodiment X, the use of either of embodiments R or S, wherein the compound is

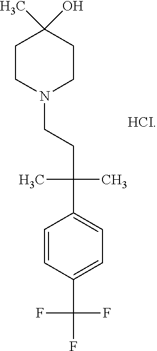 [SMILES: CC1(O)CCN(CCC(C)(C)c2ccc(C(F)(F)F)cc2)CC1 ; "HCI."]

In an embodiment Y, the use of either of embodiments R or S, wherein the compound is

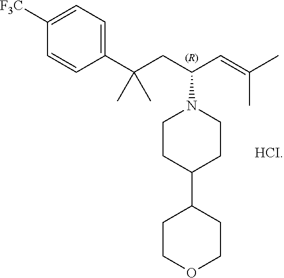

In an embodiment Z, the use of embodiment A through embodiment Y, wherein the compound is administered orally.

### EXAMPLES

Abeta oligomer preparations are know to one of skill in the art and can be found for example, in WO2015/116923 and WO2018/213281, each of which is incorporated by reference in their entirety, said methods constituting a further aspect of the disclosure. The following sigma-2 receptor modulators were used throughout the examples:

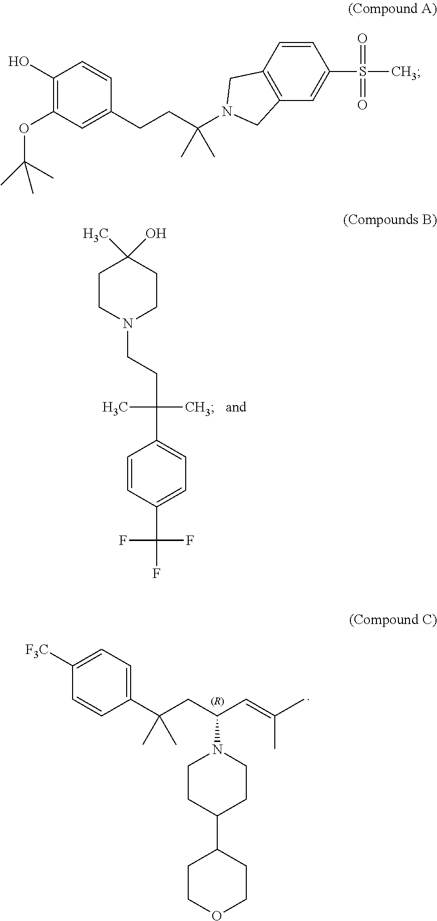

### Example 1: Testing Compounds for Treatment of Dry-AMD Associated Dysfunction

### Experimental Design:

Cell culture: Human retinal pigment epithelial cell line ARPE19 purchased from ATCC is cultured at 37° C. and 5% CO₂ in Ham's F-10 medium (Corning) supplemented with 10% fetal bovine serum (FBS) and antibiotics (media refreshed every 48-72 hours). For all experimental purposes, cells are allowed to reach confluence in the appropriate culture vessel (dependent on the assay) and then maintained in Ham's F-10 medium+5% FBS for 2 weeks prior to experimentation to ensure formation of a fully polarized epithelial monolayer. Cells are visualized by inverted microscopy throughout the experiments. Assays to detect protective activity in ocular cells were performed in accordance with Cai, H. et al., “High-Throughput Screening Identifies Compounds that Protect RPE Cells from Physiological Stressors Present in AMD,” Exp Eye Res, 185:10764 (2019), which is hereby incorporated by reference in its entirety.

Preparation of compounds: Compounds of the invention are added to cell cultures in concentrations from 10 nM to 10 μM plus vehicle to pre-incubate for 24 hrs at 37° C. and 5% CO₂.

Oxidative stress protocol: Cultures pretreated with vehicle or compounds of the invention are subjected to oxidative stress for 4 hours and 24 hours. In a second protocol, ARPE-19 cells are treated with compounds of the invention for 24 hours prior to adding the oxidative stressor tert-Butyl hydroperoxide (tBHP; 150 uM). 24 hours post-treatment, cell viability is assessed via MTT assay, propidium iodide (PI) stain, and/or lactate dehydrogenase (LDH) assay. The experimental controls for this study are: 1) vehicle (DMSO)-treated cells without an oxidative stressor (healthy vehicle control), 2) vehicle (DMSO)-treated cells treated with oxidative stressor (insult control) 3) Rescue control (e.g., Nec-7)-treated cells with oxidative stressor (full rescue of injury+control).

### Assays and Results:

Cell viability assay: The relative cell number is determined by crystal violet uptake as previously described. Cells are washed 3 times in PBS, fixed in 4% paraformaldehyde in PBS and stained in a solution of 0.1% crystal violet (Sigma Aldrich, C-3866), 10% ethanol. After washing 3 times in PBS, the remaining stain is dissolved in 10% acetic acid and absorbance is measured with a microplate reader at 540 nm. Results: Compounds of the invention decrease the degree of cytotoxicity induced by an oxidative stressor in a concentration-dependent manner.

Measurement of oxidative damage: Oxidative stress is an important factor in developing and accelerating retinal disease such as macular degeneration (Masuda T. et al., “Retinal Diseases Associated with Oxidative Stress and the Effects of a Free Radical Scavenger,” Oxidative Medicine and Cellular Longevity Vol. 2017, Article ID 9208489, (2017); and Forest, D. L., et al., “Cellular Models and Therapies for Age-Related Macular Degeneration,” Dis Model Mech 8(5): 421-427 (2015); which are hereby incorporated by reference in their entireties). Oxidative damage is measured by: 1) human 4HNE (4-hydroxynonenal) ELISA (enzyme-linked immunosorbent assay) kit for lipid peroxidation and 2) a protein carbonyl assay to detect oxidative damage to proteins. Results: Compounds of the invention decrease the degree of oxidative damage induced by an oxidative stressor in a concentration-dependent manner as measured by decreased lipid peroxidation and decrease protein carbonyl formation.

Measurement of reactive oxygen species: Cells are harvested and incubated in PBS containing 10 mM CM-H2DCFDA (the chloromethyl derivative of 2′,7′-dichlorodihydro-fluorescein diacetate; Life Technologies, D-399) for 30 min at 37° C. in the dark to allow loading of dye into the cells. This dye is nonfluorescent when chemically reduced, but after cellular oxidation and removal of acetate groups by cellular esterases it becomes fluorescent. The intracellular production of reactive oxygen species are monitored by flow cytometry with excitation at 480 nm and emission at 530 nm. Results: Compounds of the invention decrease the formation of reactive oxygen species induced by an oxidative stressor in a concentration-dependent manner as measured by decreased CM-H2DCFDA fluorescence.

Mitochondrial membrane potential (Aym): Cationic fluorescent Tetramethylrhodamine (TMRM) dye (Thermo Scientific) determines Δψm, which accumulates specifically in bioenergetically active mitochondria. The dye diffuses out of mitochondria that have lower membrane potential. Before treatment endpoint, ARPE19 cells are be loaded with TMRM (50 nM) for 30 minutes at 37° C., trypsinized, and pellets are resuspended in PBS and immediately assessed by flow cytometry (Excitation/Emission: 510/580 nm). Data is analyzed using FlowJo® software. Addition of carbonyl cyanide m-chlorophenylhydrazone CCCP will act as a positive control. Results: Compounds of the invention prevent changes in mitochondrial membrane potential induced by an oxidative stressor in a concentration-dependent manner as measured by TMRM fluorescence.

Mitochondrial mass and function assay: Estimation of mitochondrial mass is measured by loading ARPE-19 cells with MitoTracker Green dye (Excitation/Emission: 490/516 nm) at 100 nM final concentration (37° C. for 15 minutes). Cells are washed with PBS and collected after trypsinization with 0.05% trypsin. 10,000 cells per treatment are analyzed using a flow cytometer for MitoTracker fluorescence intensity. Mitochondrial function is measured using a MTT assay kit, normalizing against cell viability data to account for any cell death or proliferation. Results: Compounds of the invention prevent changes in mitochondrial mass and function induced by an oxidative stressor in a concentration-dependent manner as measured by MitoTracker Green fluorescence and MTT assay.

Autophagy flux assay: Cells are lysed in RIPA buffer (Radioimmunoprecipitation Assay Buffer, Thermo Scientific, 89901) containing protease inhibitor (Roche Applied Science, 11873580001). Cell lysates containing equal amounts of protein re-loaded in each lane and separated on a 12% SDS-PAGE gel (sodium dodecyl sulphate-polyacrylamide gel electrophoresis gel). After separation, proteins are transferred to a nitrocellulose membrane (0.22 mm; Bio-Rad Laboratories, 162-0112), and nonspecific binding sites are blocked by treating with 5% nonfat dry milk (Fisher Scientific, NC0339922) or Licor blocking buffer (Li-Cor Biosciences, 927-40000). The membranes are then incubated with primary antibodies directed against LC3B (1:1000), ATG7 (1:1000), or ATG9 (1:1000). The primary antibody treatments are followed by treatment with an horseradish peroxidase conjugated secondary antibody (for the ECL detection system) or secondary infrared dye-800 conjugated anti-rabbit dye or Alexa Fluor 680 conjugated anti-mouse IgG (for the Licor Odyssey system) for 1 hour at room temperature. To confirm equal protein loading, blots are re-probed with anti-ACTB (anti-β-actin) antibody (1:5000 dilution) or a-tubulin antibody (1:5000 dilution). For band detection, the membranes are incubated with a Western Blot detection system (ECL Plus) and exposed to single-emulsion film (Biomax MR Sigma, Z370398-50EA). Band intensities are determined using software developed by Wayne Rasband (ImageJ; National Institutes of Health, Bethesda, MD; available at http://rsb.info.nih.gov/ij/index.html). Statistical significance is calculated using the Mann-Whitney U-test. The ratio of LC3-II:LC3-I determines autophagy flux. Autophagosomes will also be detected by immunostaining. After completion of respective treatments, cells are washed 2 times in PBS and fixed in 4% paraformaldehyde for 15 minutes and subsequently immunostained with rabbit polyclonal LC3 antibody (Novus Biologicals, NB100-2220) for autophagic puncta. Fluorescence micrographs of immunostained LC3 are obtained and analyzed by Axiovision Rel 4.4 software (Zeiss, Oberkochen, Germany). The punctate staining of LC3 formed after oxidative stress indicates the formation of autophagosomes. Results: Compounds of the invention normalize changes in autophagic flux induced by an oxidative stressor in a concentration-dependent manner as measured by western blot analysis and immunofluorescence of LC3B, ATG7 and ATG9.

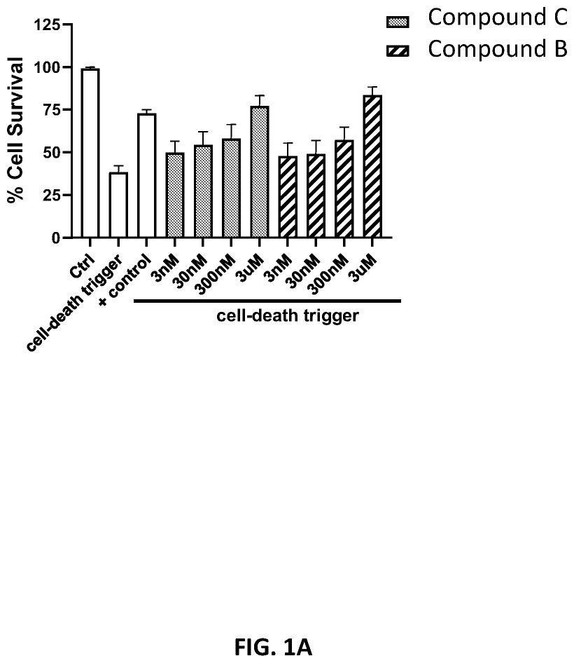

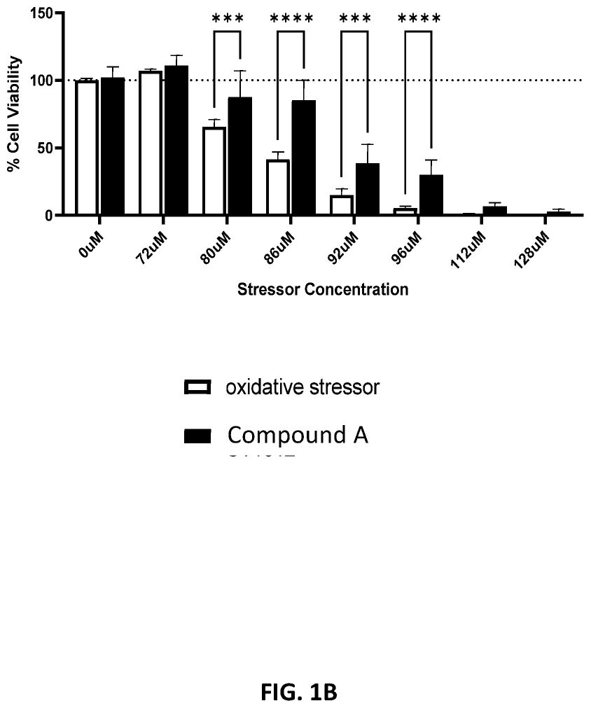

Cell death assay: Cell death is a key feature of dry AMD (Yang, M., et al., “Novel Programmed Cell Death as Therapeutic Targets in Age-Related Macular Degeneration,” Int. J. Mol. Sci., 21(19): 7279 (2020), which is hereby incorporated by reference in its entirety). A functional cell-based assay in retinal pigment epithelium cells (RPEs) was performed to assess cell survival after oxidative stress (cell-death trigger). The data demonstrate that sigma-2 receptor modulators rescue cell death triggered by oxidative stress in a concentration-dependent manner (FIGS. 1A and 1B).

Determining lysosomal integrity and activity: Retinal pigment epithelial cells are plated in 8-well coverglass bottom chambers (Lab-Tek, Naperville, IL) and cells are incubated in 1 mg/ml of the pH indicator LysoSensor Yellow/Blue dextran (Molecular Probes, Eugene, OR) for 12 hours in the presence or absence of varying oxidative stressors. The labelled cells are observed with a laser scanning confocal microscope using excitation at 360 nm and an emission filter at 450 nm and a long pass emission filter at 515 nm. Higher 530/450 nm ratios (i.e. a shift to green) correlate with a lower pH. Cathepsin D activity is measured in cell lysates using a fluorometric cathepsin D activity assay kit (Abcam, Cambridge, MA) and values are presented as relative fluorescence units per million cells. Results: Compounds of the invention protect against loss of lysosomal integrity and activity induced by an oxidative stressor in a concentration-dependent manner as measured by LysoSensor Yellow/Blue fluorescence.

### Example 2: In Vitro Models of Retinal Pigment Epithelial Cells

Spontaneously arising retinal pigment epithelial (RPE) cells, such as ARPE-19 cells, are cultured as described in Example 1. In brief, aged cells grown on trans-well inserts that allow for mature, pigmented RPE monolayers to emulate several aspects of in-situ/ocular RPE cells (ultrastructure, physiology and function) are used in experiments to test compounds of the invention for efficacy in preventing or treating cellular dysfunction.

Human pluripotent stem cell (iPSC)-derived RPE lines are highly characterized and are considered mature pigmented monolayers with intact barrier functions and physiological functions of RPEs (such as, vascular endothelial growth factor (VEGF) secretion), are used in experiments to test compounds of the invention for efficacy in preventing or treating cellular dysfunction.

Primary RPE cells from healthy donors and AMD donors are the most physiologically and pathophysiologically relevant cell model for study of AMD-relevant phenotypes and functions and are used in experiments to test compounds of the invention for efficacy in preventing or treating cellular dysfunction.

### Example 3: In Vitro Modulation of Pathways Important in Dry AMD

Proof-of-concept studies indicate a clear role of sigma-2 receptor modulators in rescuing key aspects of dry AMD. Mechanistic studies and pathway analysis in a disease-relevant assay suggest a key role of sigma-2 receptor modulators in dry AMD as studied in RPE cells (Table 1).

Pathways Altered by Compound A vs Vehicle (p-value < 0.05)

| | |
|---|---|
| Oxidative Stress | |
| | Apoptosis and survival APRIL and BAFF signaling |
| | Signaling transduction role of MIF as an intracullar mediator |
| | Cell cycle role of SCF complex in a cell cycle regulation |
| | Immune response INF-alpha/beta signaling via JAK/STAT |
| | Development PEDF signaling |
| Aβ Oligomers | |
| | Immune response BAFF-induced non-canonical NF-κB signaling |
| | Immune response role of PKR in stress-induced antiviral cell |
| | response |
| | Apoptosis and survival APRIL and BAFF signaling |
| | Apoptosis and survival NGF activation of NF-κB |
| | Apoptosis and survival Apoptotic TNF-family pathways |
| Inflammation | |
| | Cytoskeleton remodeling regulation of actin cytoskeleton |
| | nucleation and polymerization by Rho GTPases |
| | Neurophysiological process activity-dependent synaptic AMPA |
| | receptor removal |
| | Cell adhesion classical cadherin-mediated cell adhesion |
| | Transcription ligand-dependent activation of the ESR1/SP pathway |
| | Immune response lysophosphatidic acid signaling via NF-κB |

Oxidative Stress: Oxidative stress is a key aspect of AMD and oxidative insult leads to defects in photoreceptor outer segment (POS) trafficking in RPE cells, which results in accumulation of toxic macromolecules as well as disruption of the autophagy-lysosomal pathway and cellular proteostasis. Hydrogen peroxide (H₂O₂) is a mediator of oxidative stress in human vitreous and therefore, is used on RPE monolayers. RPE monolayers are exposed to varying concentrations of oxidative stressors to model oxidative stress that is present in AMD as well as in the aging retina. Cultures are pre-treated with oxidative stressors and the ability to traffic and degrade fluorescently tagged POS is assessed, including measurement of late compartments critical for cargo degradation, such as lysosomes and autophagy bodies. Cells are tested in the presence or absence of the compounds of the invention. Compounds of the invention decrease the degree of oxidative damage induced by oxidative stressors in a concentration-dependent manner.

Complement Inhibition: Complement dysregulation is a major contributing factor genetically linked to approximately half of all AMD cases. Complement C3 is a critical upstream component of the complement cascade. C3 turnover is increased significantly in donor RPE cells from AMD patients carrying the complement factor H Y402H polymorphism, resulting in higher internalisation and deposition of the terminal complex C5b-9 in lysosomes. Studies of RPE cells in Stargardt disease, which shares similarities with AMD, reveal enlarged endosomes that traffic C3. To mimic complement dysregulation in vitro, we target complement C3 activity. Cultured RPE cells are exposed to cobra venom, which recapitulates disruption of the complement pathway, by targeting C3. CD59 recycling and lysosome exocytosis after complement attack is defective in this model, and fails to protect RPE cells. Evidence indicates that complement dysregulation disrupts cargo trafficking and processing in the RPE. In this model, cultures are exposed to snake venom and subsequently photoreceptor outer segments (POS) are added to the medium with and without the presence of the compound and extent of rescue determined. Photoreceptor outer segments are prepared as per Ratnayaka J A, Keeling E, Chatelet D S: Study of Intracellular Cargo Trafficking and Co-localization in the Phagosome and Autophagy-Lysosomal Pathways of Retinal Pigment Epithelium (RPE) Cells. Methods Mol Biol 2020; 2150:167-82. Compounds of the invention rescue cell death and deficits in trans-epithelial electrical resistance (TEER) of RPE barriers.

Aβ Oligomer Exposure: As mediates pathogenesis in RPE cells characterized by deficits in photoreceptor outer segment trafficking, barrier (tight junction) integrity, and cellular health. RPE cultures are treated with human oligomeric Aβ in either the presence or absence of compounds of the invention. POS trafficking and barrier integrity are measured and cellular health is determined with a lactate dehydrogenase (LDH) assay. Compounds of the invention rescue deficits in photoreceptor outer segment trafficking, barrier integrity, and cellular health that are triggered by oligomeric Aβ.

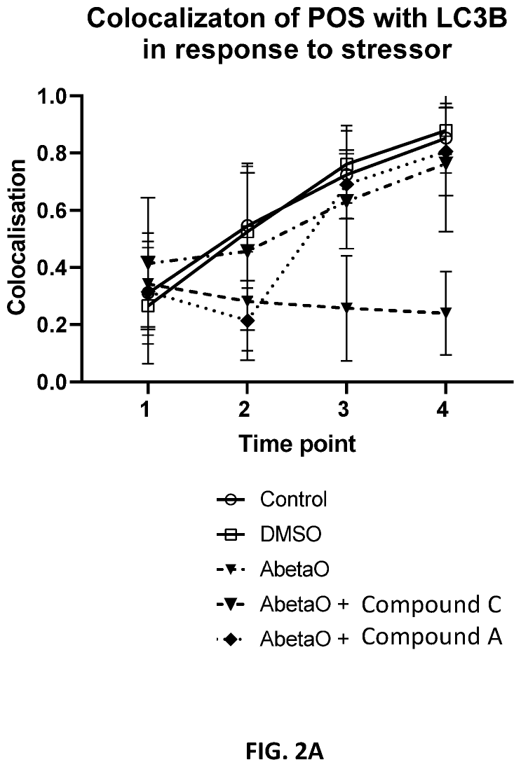

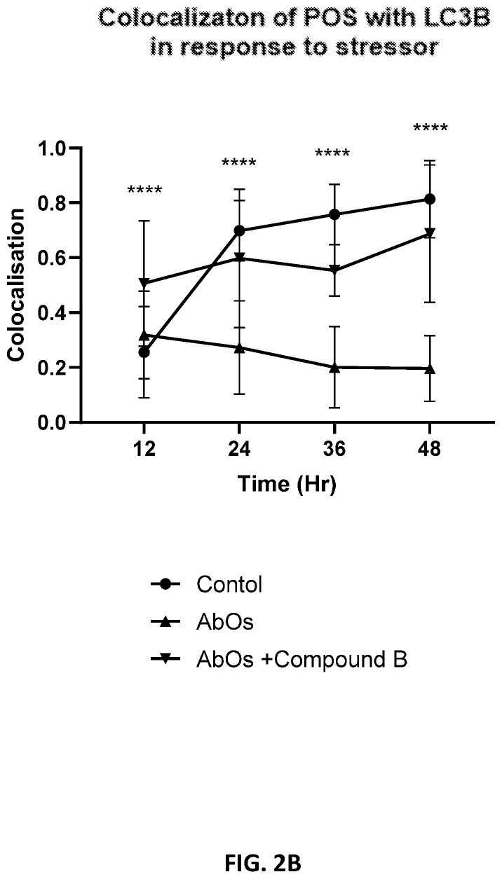

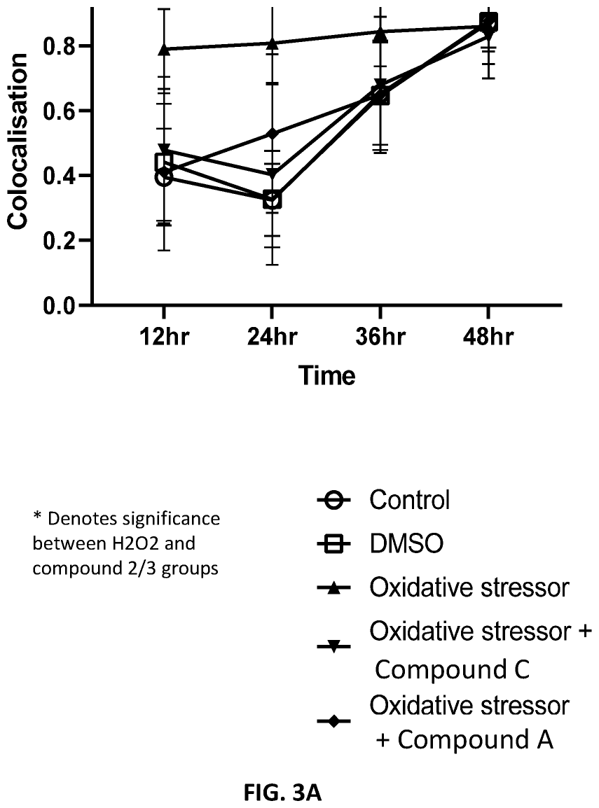

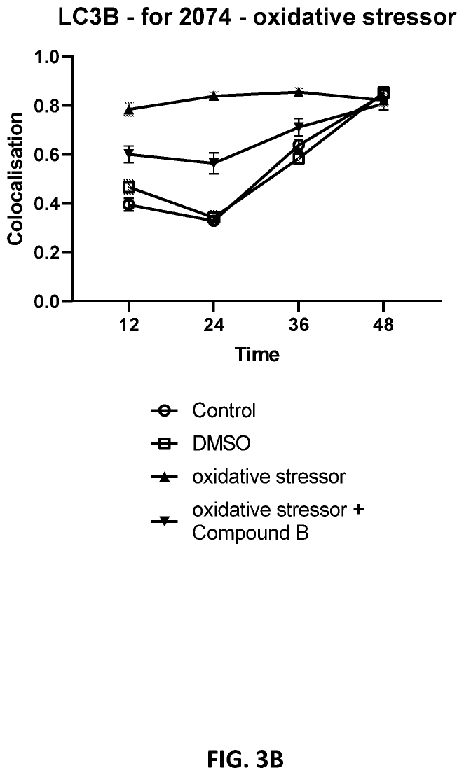

Autophagy Assays: Autophagy assays were performed in accordance with Klionsky et al., “Guidelines for the Use and Interpretation of Assays for Monitoring Autophagy,” (4th edition) Autophagy 2021; 17:1-382, which is hereby incorporated by reference in its entirety. RPE cultures were treated with human oligomeric Aβ in either the presence or absence of compounds of the invention. POS trafficking was measured over time after the addition of POS to RPE cell cultures. Stressors included addition of Aβ oligomers or H₂O₂ for 12 to 48 hours. These studies indicated that POS are trafficked over time at a normal rate after the addition of POS to RPE cell cultures. This process was disrupted by addition of stressors such as Aβ oligomers (FIGS. 2A and 2B) or oxidative stress (FIGS. 3A and 3B). The presence of sigma-2 receptor modulators (Compound A and Compound C) restored normal trafficking into autophagosomes after stress (FIGS. 2A and 2B and FIGS. 3A and 3B) as measured by colocalization of POS with microtubule-associated protein 1 light chain 3B (LC3B) after stress.

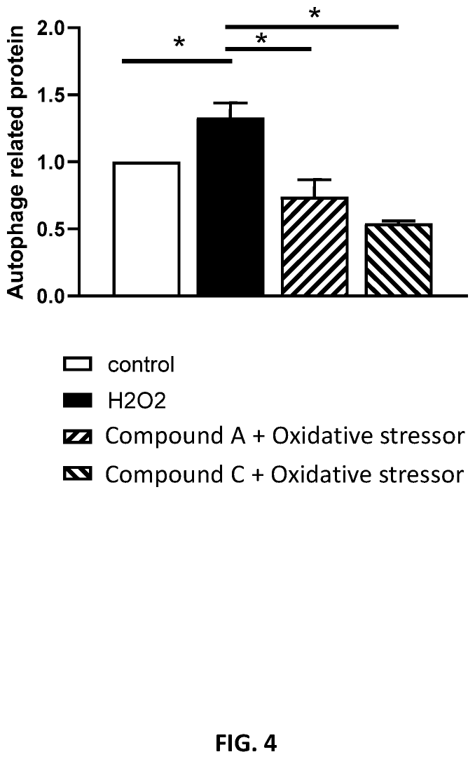

Oxidative stressors are associated with pathology in AMD with changes seen in autophagy associated proteins. Application of an oxidative stressor caused increased expression of an autophagy related protein acutely (FIG. 4). Sigma-2 receptor modulators Compound A and Compound C prevented the increase in this autophagy related protein (FIG. 4).

### Example 4. Prevention of Senescence of Retinal Pigment Epithelial Cells

Experimental Design: Primary RPE cultures or ARPE-19 cells are treated with non-lethal CSC (Cigarette Smoke Condensate; 150 uM) to cause senescence of RPE cells in the presence or absence of compound of the invention for 6 to 10 days. 6 to 10 days following treatment, cell senescence is assessed via b-galactosidase immunocytochemistry. Experimental Controls: 1) vehicle (DMSO)-treated cells without CSC (healthy vehicle control), 2) vehicle (DMSO)-treated cells treated with CSC (senescence+control) 3) Rescue+control (e.g., mitochondrial DRP1 mutant protein with a virus-treated cells with CSC) (partial rescue of senescence+control). Experimental Results: Compounds of the invention prevent RPE cell senescence triggered by administration of CSC.

### Example 5. Prevention of Inflammatory Response in Retinal Pigment Epithelial Cells

Experimental Design THP-1 cells are treated with media from cultured RPE cells that have undergone oxidative stress to induce expression of proinflammatory markers in the THP-1 cells. Prior to THP-1 cell treatment, RPE cells (+/−oxidative stress) are treated with a compound of the invention to prevent a pro-inflammatory response in the THP-1 cells. Experimental Controls: 1) vehicle (DMSO)-treated cells (healthy vehicle control), 2) vehicle (DMSO)-treated RPE derived medium (THP+control). Experimental Results: Compounds of the invention prevent a pro-inflammatory response in THP cells triggered by administration of RPE derived medium.

### Example 6. Prevention of Apoptosis of Retinal Ganglion Cells

Experimental Design: In an optic nerve crush model, immunopanned retinal ganglion cells (RGCs) are cultured with increasing concentrations of compounds described herein. Caspase 3 activity and cell viability are assessed in the retinal ganglion cells by calcein-AM staining. Experimental Results: Compounds of the invention prevent retinal ganglion cell apoptosis.

### Example 7. Prevention of Ocular Hypertension

Experimental Design: An increase in intraocular pressure is induced in rats by application of light from a diode laser to the trabecular meshwork in the presence or absence of a compound of the invention. Axons in the optic nerve are counted 24 hours following induction of ocular hypertension. Experimental Results: Compounds of the invention prevent ocular hypertension-induced cell death and prevent axonal injury-induced functional deficits.

### Example 8: In Vivo Penetration of Retina

Orally administered compounds of the invention penetrate the retina at concentrations exceeding 80% occupancy of sigma-2 receptors.

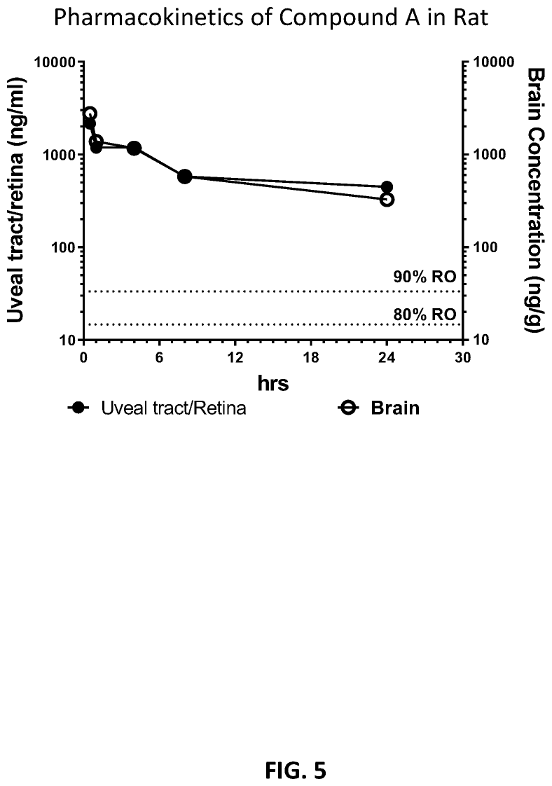

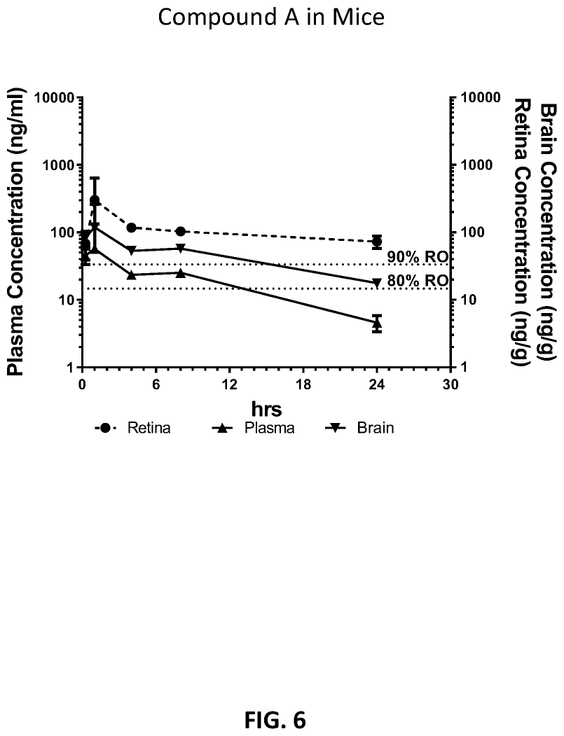

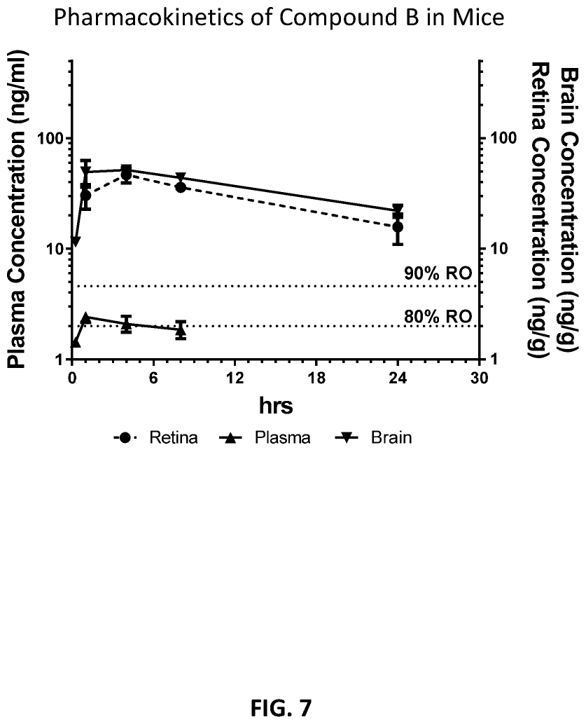

Following a single oral administration of [14C]-Compound A to a rat, autoradiography was performed to determine tissue distribution. [14C]-Compound A was found in concentrations that exceeds 80% receptor occupancy at the sigma-2 receptor in the uveal tract/retina over the entire course of 24 hrs, and these concentrations were comparable to that in brain (FIG. 5). Receptor occupancy exceeding 80% confers efficacy in vivo. Drug concentration in the brain (cerebellum), retina, and plasma were also measured. The concentration in the retina exceeded 80% receptor occupancy at sigma-2 receptors over the entire course of 24 hrs and was higher than the concentration in the brain or plasma (FIG. 6). Similarly, following a single orally administered dose, Compound B was found in concentrations that exceeded 80% receptor occupancy at sigma-2 receptors in the retina and plasma and approximately 50% occupancy in the brain (FIG. 7).

### Example 9: Sigma-2 Receptor Modulators in Glaucoma

In vivo models of glaucoma (Shah, M., et al., “Translational Preclinical Pharmacologic Disease Models for Opthalmic Drug Development,” Development. Pharm. Res. 36(4): 58 (2019); which is hereby incorporated by reference in its entirety) were used to test the utility of sigma-2 receptor modulators.

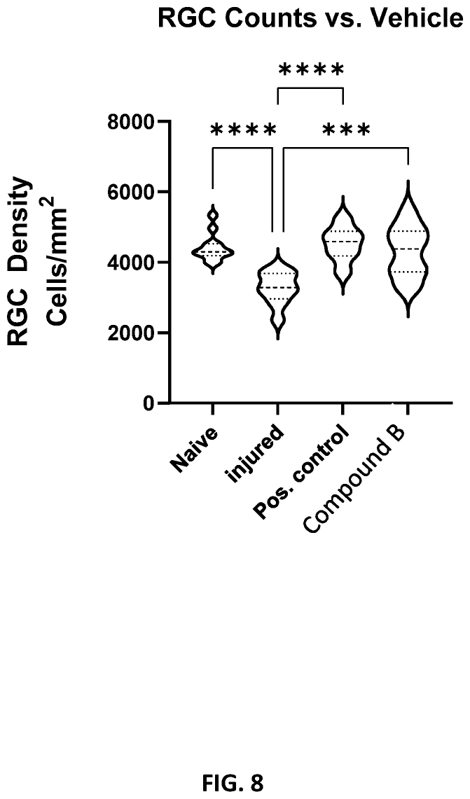

To test whether sigma-2 receptor modulators can rescue cell death, in vivo models were used to cause cell death of retinal ganglion cells, resembling what is seen in patients with glaucoma and other retinal diseases. Addition of the sigma-2 receptor modulator Compound B significantly preserved retinal ganglion cells counts (p<0.05) similar to benchmark positive controls (FIG. 8).

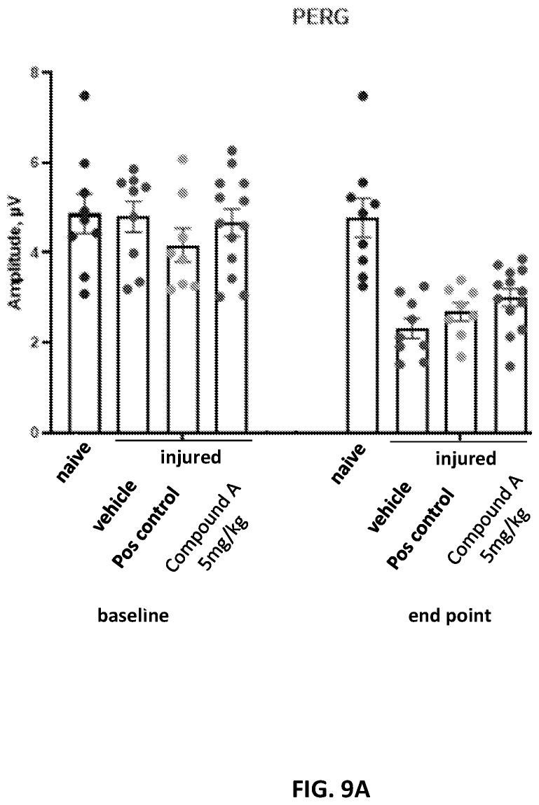

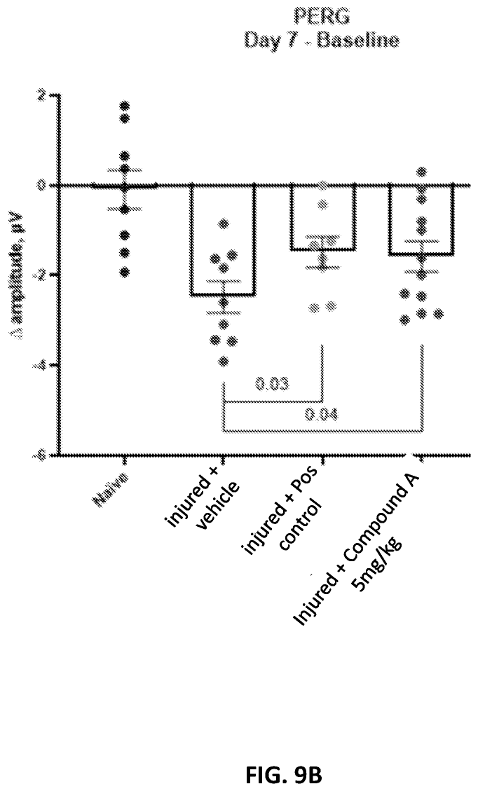

Glaucoma can cause perturbations in the electrical activity of the retina. To measure the electrical activity, a pattern electroretinogram (PERG) measure electrical activity of the retina in response to a test stimulus, such as a reversing checkerboard. PERG is a noninvasive, direct, and objective method to assess retinal ganglion cell function. An in vivo model of glaucoma causes a functional deficit in retinal ganglion cells (injured vs. naïve), resembling what is seen in patients with glaucoma and other retinal diseases. A sigma-2 receptor modulator Compound A preserves retinal ganglion cell function (FIGS. 9A and 9B; p<0.05).

### Example 10: Disease Indications

Unbiased pathway analysis of proteomic data from obtained during clinical trials provides evidence of the relationship between the sigma 2 receptor complex and dry AMD. Analyses of cerebral spinal fluid (CSF) were performed to ascertain which predesignated functional disease ontologies may be affected by the administration of Compound A. These analyses identified geographic atrophy and macular degeneration as two of the top indications affected (Table 2). Subsequent analyses identified several subsets of proteins altered by Compound A that are involved in dry AMD.

TABLE 2
Top Disease Ontologies
Top Disease Ontologies

Geographic atrophy
Central nervous system diseases
Cognition disorders
Mental disorders
Psychiatry and psychology
Macular degeneration
Neurocognitive disorders
Rett Syndrome
Dementia
Movement disorders
Neurodegenerative diseases
Brain diseases
Basal ganglia diseases
Anemia
Infections

In subsequent analyses examining the overlap of proteins altered in CSF and plasma biofluids of AD patients treated with Compound A versus placebo, a set of proteins were identified that are altered by Compound A. These proteins have been previously shown by other groups to be disrupted in dry AMD or geographic atrophy, compared to age-matched controls. Subsequent analysis identified several pathways in which these proteins are involved, many of which have known genetic or biological links to processes disrupted in dry AMD. The collective insights provided by these analyses provide early proof of concept that a sigma-2 receptor modulator is capable of altering AMD relevant proteins and pathways in an aged patient population.

### Synthetic Examples

Compounds according to any embodiment described herein, may be prepared by the general and specific methods outlined in, for example, WO2013/029057, WO2015/116923 and WO2018/213281, each of which is incorporated by reference in their entirety, said methods constituting a further aspect of the disclosure.

1. A method of treating dry age-related macular degeneration (dry AMD), comprising administering to a subject in need thereof, a therapeutically effective amount of a compound selected from the group consisting of:
A. a compound of Formula I, 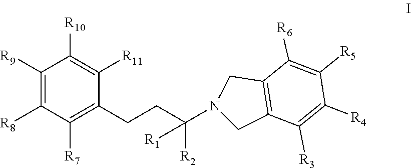
or a pharmaceutically-acceptable salt thereof:
wherein:
each of R₁ and R₂ is independently selected from H, C₁-C₆ alkyl, or CH₂OR′; wherein each R′ if present in R₁, and R₂ is independently H or C₁-C₆ alkyl;
each of R₃, R₄, R₅, and R₆ is independently selected from the group consisting of H, C₁-C₆alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), F, Cl, Br, I, CF₃, CN, NO₂, NH₂, C₁-C₆ haloalkyl, C₁-C₆ hydroxyalkyl, C₁-C₆ alkoxy C₁-C₆alkyl, aryl, heteroaryl, C₃-C₇ cycloalkyl, heterocycloalkyl, alkylaryl, CO₂R′, C(O)R′, NH(C₁-C₄ alkyl), N(C₁-C₄ alkyl)₂, NH(C₃-C₇ cycloalkyl), NHC(O)C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₃, R₄, R₅, and R₆ is independently selected from the group consisting of H, CH₃, CH₂CH₃, C₃-C₆ alkyl, C₁-C₆ haloalkyl, or optionally substituted aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂, wherein the optionally substituted group is selected from C₁-C₆ alkyl or C₂-C₇ acyl;
or R₃ and R₄, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₃ and R₄ are linked together to form a —O—C₁-C₂ methylene-O— group;
or R₄ and R₅, together with the C atom to which they are attached form a 4-, 5-, 6-7- or 8-membered cycloalkyl, aryl, heteroaryl, or heterocycloalkyl that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₄ and R₅ are linked together to form a —O—C₁₋₂ methylene-O— group;
each of R₇, R₈, R₉, R₁₀, and R₁₁ is independently selected from the group consisting of H, C₁-C₆ alkyl, OH, OCH₃, OCH(CH₃)₂, OCH₂CH(CH₃)₂, OC(CH₃)₃, O(C₁-C₆ alkyl), OCF₃, OCH₂CH₂OH, O(C₁-C₆ alkyl)OH, O(C₁-C₆ haloalkyl), O(CO)R′, F, Cl, Br, I, CF₃, CN, NO₂, NH₂, C₁-C₆ haloalkyl, C₁-C₆ hydroxyalkyl, C₁-C₆ alkoxy C₁-C₆ alkyl, aryl, heteroaryl, C₃-C₇ cycloalkyl, heterocycloalkyl, alkylaryl, heteroaryl, CO₂R′, C(O)R′, NH(C₁-C₄ alkyl), N(C₁-C₄ alkyl)₂, NH(C₃-C₇ cycloalkyl), NHC(O)(C₁-C₄ alkyl), CONR′₂, NC(O)R′, NS(O)ₙR′, S(O)ₙNR′₂, S(O)ₙR′, C(O)O(C₁-C₄ alkyl), OC(O)N(R′)₂, C(O)(C₁-C₄ alkyl), and C(O)NH(C₁-C₄ alkyl); wherein each R′ if present in R₇, R₈, R₉, R₁₀, and R₁₁ is independently selected from the group consisting of H, CH₃, CH₂CH₃, C₃-C₆ alkyl, C₁-C₆ haloalkyl, aryl, alkylaryl, piperazin-1-yl, piperidin-1-yl, morpholinyl, heterocycloalkyl, heteroaryl, C₁-C₆ alkoxy, NH(C₁-C₄ alkyl), and N(C₁-C₄ alkyl)₂;
or R₇ and R₈, together with the N or C atoms to which they are attached form a 4-, 5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₇ and R₈ are linked together to form a —O—C₁₋₂ methylene-O— group;
or R₈ and R₉, together with the N or C atoms to which they are attached form a 4-5-, 6-7- or 8-membered cycloalkyl, aryl, heterocycloalkyl or heteroaryl group that is optionally substituted with 1, 2, 3, 4, or 5 substituents independently selected from OH, amino, halo, C₁-C₆ alkyl, C₁-C₆ haloalkyl, C₁-C₆ alkoxy, C₁-C₆ haloalkoxy, aryl, arylalkyl, heteroaryl, heteroarylalkyl, cycloalkyl, and heterocycloalkyl or R₈ and R₉ are linked together to form a —O—C₁₋₂ methylene-O— group;
each n is independently 0, 1, or 2;
with the proviso that R₇, R₈, R₉, R₁₀, and R₁₁ are not all H; and
with the proviso that the following compounds, or pharmaceutically acceptable salts thereof are excluded: 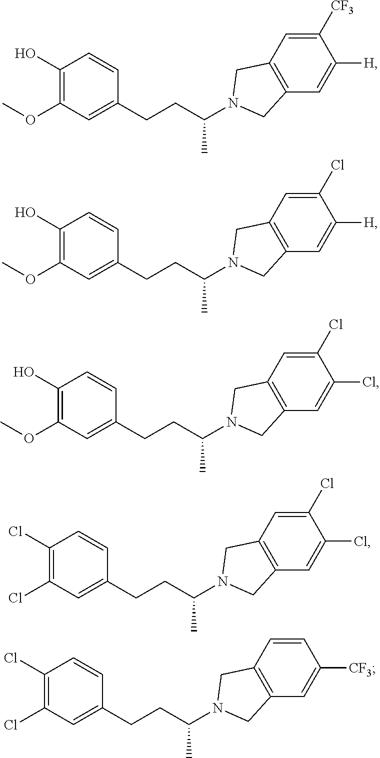
or
B. a compound of Formula IA 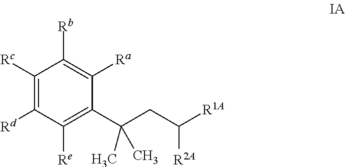
or pharmaceutically acceptable salt thereof:
wherein:
each of R^{a}, R^{b}, R^{c}, R^{d} and R^{e} is independently selected from the group consisting of, H, hydroxyl, Cl, F, methyl, —OCH₃, —OC(CH₃)₃, O—CH(CH₃)₂, CF₃, SO₂CH₃, and morpholino;
R^{2A} is selected from the group consisting of hydrogen, alkyl, phenyl, or —CH═C(CH₃)₂; and
R^{2A} is an optionally substituted cyclic amino group.

2. The method of claim 1, wherein the compound is a compound of Formula I or a pharmaceutically-acceptable salt thereof.

3. The method of claim 1, wherein the compound is 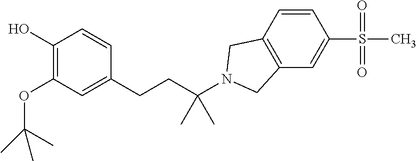 [SMILES: CC(C)(C)Oc1cc(CCC(C)(C)N2Cc3ccc(S(C)(=O)=O)cc3C2)ccc1O]
or a pharmaceutically acceptable salt thereof.

4. The method of claim 1, wherein the pharmaceutically acceptable salt is selected from the group consisting of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

5. The method of claim 1, wherein the pharmaceutically acceptable salt is the fumarate salt.

6. The method of claim 1, wherein the compound is

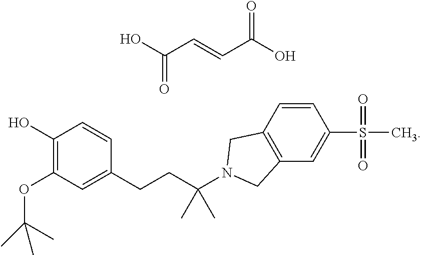 [SMILES: O=C(O)/C=C/C(=O)O ; CC(C)(C)Oc1cc(CCC(C)(C)N2Cc3ccc(S(C)(=O)=O)cc3C2)ccc1O]

7. The method of claim 1, wherein the compound is a compound of Formula IA or a pharmaceutically-acceptable salt thereof.

8. The method of claim 1, wherein the R^{2A} is optionally substituted piperidinyl.

9. The method of claim 1, wherein the R^{2A} is selected from the group consisting of

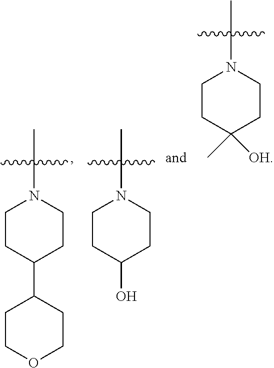

10. The method of claim 1, wherein the compound is selected from the group consisting of 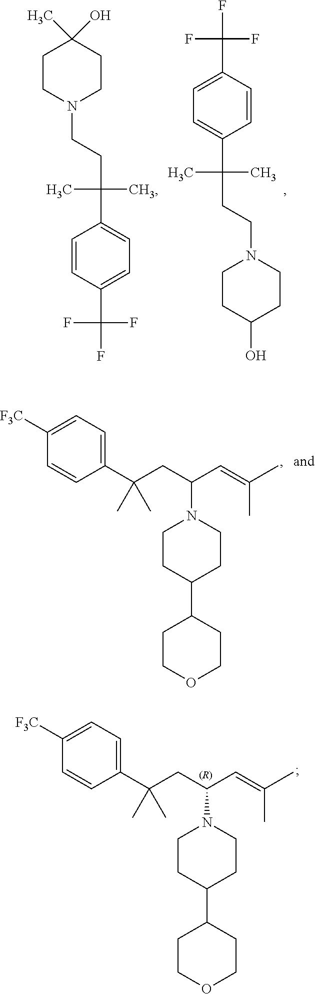
or a pharmaceutically acceptable salt thereof.

11. A method of treating dry age-related macular degeneration dry (AMD), comprising administering to a subject in need thereof, a therapeutically effective amount of a compound selected from the group consisting of:

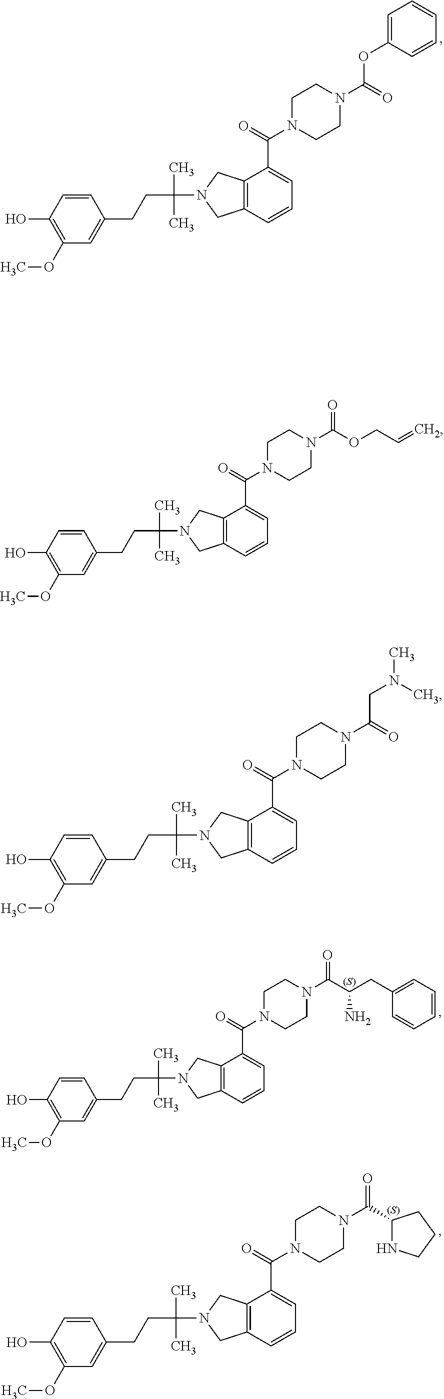

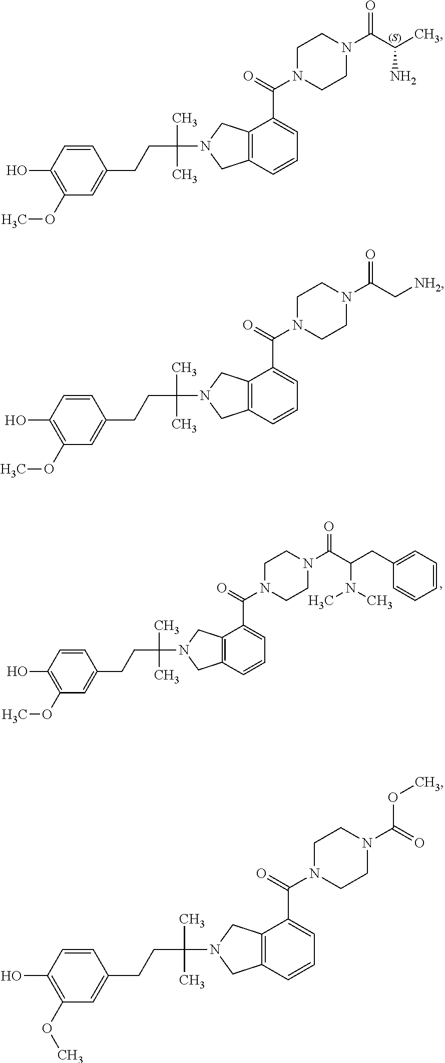

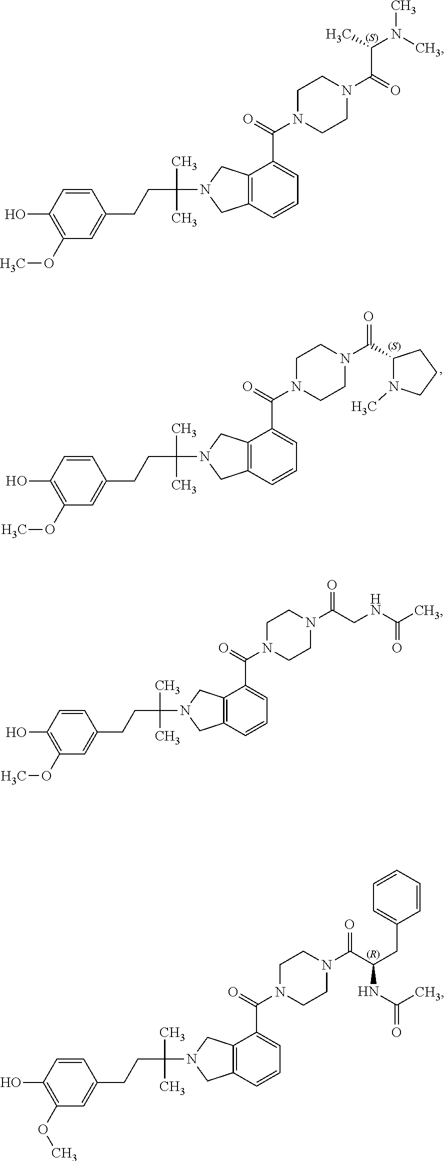

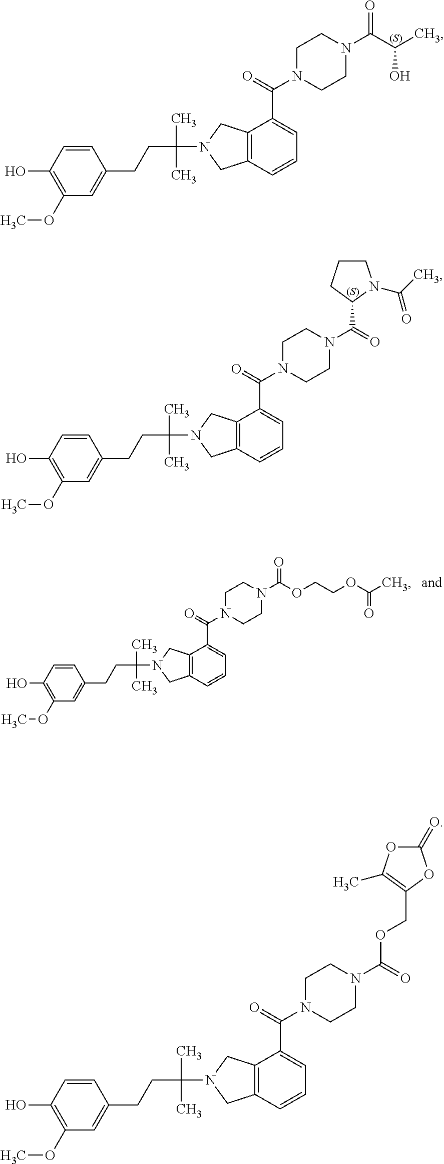

12. A method of treating dry age-related macular degeneration comprising administering to a subject in need thereof, a therapeutically effective amount of a pharmaceutical composition comprising a compound according to claim 1 and a pharmaceutically acceptable excipient.

13. A method of treating dry age-related macular degeneration comprising administering to a subject in need thereof, a therapeutically effective amount of a pharmaceutical composition comprising a compound selected from the group comprising: 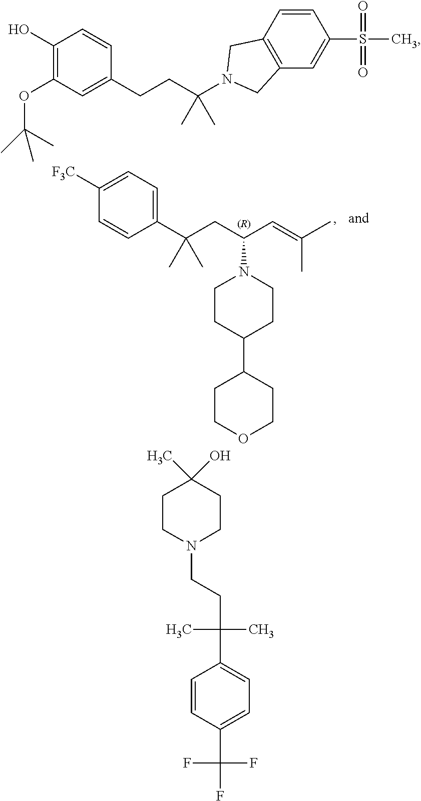
or a pharmaceutically acceptable salt thereof, and a pharmaceutically acceptable excipient.

14. The method of claim 13, wherein the pharmaceutically acceptable salt is selected from the group consisting of hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

15. The method of claim 13, wherein the pharmaceutically acceptable salt is the fumarate salt.

16. The method of claim 15, wherein the compound is selected from the group consisting of 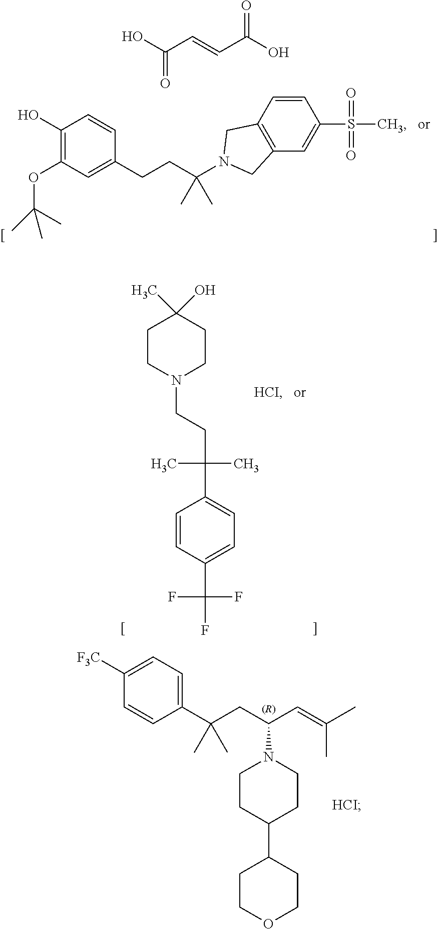
or a pharmaceutically acceptable salt thereof.

17.-25. (canceled)
